(19) United States
(12) Patent Application Publication
(10) Pub. No.: US 2022/0213138 A1
(43) Pub. Date: Jul. 7, 2022

(54) CYCLIC COMPOUNDS AND METHODS OF MAKING AND USING

(71) Applicant: UNIVERSITY OF HONG KONG, Hong Kong (CN)

(72) Inventors: Xuechen Li, Hong Kong (CN); Yue Zhang, Hong Kong (CN)

(21) Appl. No.: 17/600,922

(22) PCT Filed: Apr. 4, 2019
(86) PCT No.: PCT/IB2019/052790

§ 371 Date: Oct. 1, 2021

(51) Int. Cl. C07K 1/113; C07K 7/64

(52) U.S. Cl. CPC C07K 1/113; C07K 7/64

(57) ABSTRACT

Disclosed are compounds and methods for highly effective chemoselective peptide cyclization and bicyclization directly on unprotected peptides and other compounds as well as the compounds produced by the methods, which have a novel structural motif. The fast reaction rate and operational simplicity render this method to be highly effective to synthesize cyclic structures, i.e. cyclic peptides. The cyclic compounds allow for various functionalities useful in chemical biology study and drug discovery.

### REFERENCE TO SEQUENCE LISTING

The Sequence Listing submitted on Apr. 4, 2019, as a text file named “UHK_00833_PCT_ST25.txt,” created on Mar. 29, 2019, and having a size of 12,161 bytes is hereby incorporated by reference pursuant to 37 C.F.R. § 1.52(e)(5).

### FIELD OF THE INVENTION

The invention is generally directed to cyclic compounds and methods for making and using them, more particularly to cyclic peptides and methods for making and using them.

### BACKGROUND OF THE INVENTION

Peptide cyclization confers peptides with more rigid conformation (Morrison, Nature Reviews Drug discovery, 17(8):531-533 (2018); Driggers, et al., Nature Reviews Drug Discovery, 7(7):608 (2008); Wang, et al., Nature Chemical Biology, 14(5):417 (2018)) and enhanced stability towards enzymatic proteolysis (Tapeinou, et al., Peptide Science, 104(5):453-461 (2015); Kessler, Angew. Chem. Int. Ed. Engl., 21(7):512-523 (1982)). Many cyclic peptides have been discovered from different kingdoms of organisms, which exhibit diverse biological activities, including anti-tumor, antimicrobial, and anti-inflammatory activities (Wang, et al., Nature Chemical Biology, 14(5):417 (2018); Kritzer, Nature Chemical Biology, 6(8):566 (2010); Kohli, Nature, 418(6898):658 (2002)). The rigidity of cyclic peptides can lower the entropic cost of the Gibbs free energy when engaged in large binding surface. As a result, cyclic peptides are being used to probe and disturb protein-protein interaction (PPIs) (Rubin, et al., Crit. Rev. Eukaryot. Gene Expr., 26(3):199-221 (2016)), which are considered as “undruggable” targets in conventional small-molecule based drug discovery. Based on the structures, cyclic peptides can be classified into head-to-tail, head-to-side chain, side chain-to-tail and side chain-to-side chain cyclization. Various methods and strategies have been developed to construct cyclic peptides (White, et al., Nature Chemistry, 3(7):509 (2011)). In particular, advances in effective chemoselective methods enable cyclization directly on unprotected native peptides. For example, Pentelute and co-workers used palladium-mediated lysine or cysteine arylation (Spokoyny, et al., J. Am. Chem. Soc., 135(16):5946-9 (2013); Rojas, et al., Chem. Sci., 8(6):4257-4263 (2017); Lee, et al., Angew. Chem. Int. Ed. Engl., 56(12):3177-3181 (2017); Vinogradova, et al., Nature, 526(7575):687 (2015); Zhang, et al., Nature Chemistry, 8(2):120 (2016)), and Dawson and co-workers used dichloroacetone (Assem, et al., Angew. Chem. Int. Ed. Engl., 54(30):8665-8668 (2015)), Chou used thiol-ene reaction (Wang, et al., Angew. Chem. Int. Ed. Engl., 54(37):10931-10934 (2015)) and Greenbaum used dibromo-m-xylene (Jo, et al., J. Am. Chem. Soc., 134(42):17704-17713 (2012)) to form Cys-Cys dialkylation cyclization. In Dawson's case, the cyclization provided the exocyclic carbonyl functional group for further late-stage conjugation via oxime reactions (Assem, et al., Angew. Chem. Int. Ed. Engl., 54(30):8665-8668 (2015)).

The three-component coupling reaction of ortho-phthalaldehyde (OPA), a thiol moiety (i.e., 2-mercaptoethanol) and an amine to form the fluorescent 1-substituted-thio-2-substituted-isoindole, which had historically been used to detect the amino group during peptide Edman degradation process and analytical determination of amino acids. Roth first reported this reaction and its use for fluorometric detection of amino acids in 1971 (Roth, Anal. Chem., 43(7):880-882 (1971)). Benson and co-workers later improved the reaction reproducibility by using a large excess of 2-mercaptoethanol and adding Brij (Benson, et al., Proc. Natl. Acad. Sci. U.S.A., 72(2):619-22 (1975)). The established protocol for this three-component reaction required premixing of large excess of both mercaptoethanol and OPA prior to addition of the amino acid in boric acid buffer (pH 9.7) in order to achieve clean and reproducible results (Simons, et al., Anal. Biochem., 82(1):250-4 (1977)). Mechanistically, this reaction is still not clear to explain the phenomena.

There remains a need to develop functional cyclic compounds useful in drug discovery and chemical biology study. There is also a need for an effective method for making cyclic compounds that is rapid, clean, simple, and allows for diverse functionalities.

Therefore, it is the object of the present invention to provide functional cyclic compounds.

It is another object of the present invention to provide methods of making such compounds.

It is another object of the present invention to provide methods of using such compounds.

It is yet another object of the present invention to provide kits for synthesizing such compounds.

Throughout the description and claims of this specification, the word “comprise” and variations of the word, such as “comprising” and “comprises,” mean “including but not limited to,” and are not intended to exclude, for example, other additives, components, integers or steps.

Any discussion of documents, acts, materials, articles or the like which has been included in the present specification is not to be taken as an admission that any or all of these matters form part of the prior art base or were common general knowledge in the field relevant to the present disclosure as it existed before the priority date of each claim of this application.

### SUMMARY OF THE INVENTION

Disclosed are cyclic compounds and methods for making and using them. In particular, disclosed are cyclic compounds having a structure of Formula I:

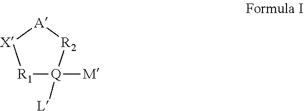

where A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;

where X′ is —NR₃, an oxygen atom, or a sulfur atom, where R₃ is a hydrogen, a substituted alkyl group, a substituted cycloalkyl group, a substituted heteroalkyl group, a substituted alkenyl group, a substituted heteroalkenyl group, a substituted aryl group, or a substituted heteroaryl group;

where R₁ and R₂ are independently absent, an unsubstituted alkyl group, a substituted alkyl group, an unsubstituted heteroalkyl group, or a substituted heteroalkyl group;

where Q is a compound of interest; and

where L′ and M′ are independently absent or a compound of interest.

In some forms, X′ is a sulfur atom. In form forms, Q, L′, and M′, if present, are the same type of molecule, such as the same type of oligomer. In some forms, Q can be an oligomer or a synthetic material. In some forms, Q can be an oligomer of synthetic monomer residues. In some forms, L′ and M′ are one or more monomer residues or a synthetic material. In some forms, L′ and M′ are one or more monomer residues. In some forms, L′ and M′ are a synthetic material.

In some forms, the monomer residues can each be independently amino acid residues or nucleotide residues. In some forms, the monomer residues can be amino acid residues. In some forms, the monomer residues can be nucleotide residues. In some forms, L′ and M′ can each be independently one or more amino acid residues.

In some forms, Q can be a peptide or an oligonucleotide. In some forms, Q can be a peptide. In some forms, Q can be a linear peptide, a cyclic peptide, or a branched peptide. In some forms, Q can be a linear peptide. In some forms, Q can be a cyclic peptide. In some forms, Q can be a branched peptide. In some forms, Q can be an oligonucleotide. In some forms, Q can be an unprotected peptide. In some forms, Q can be an oligonucleotide.

In some forms, A′ is

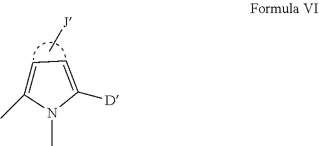

where J′ is

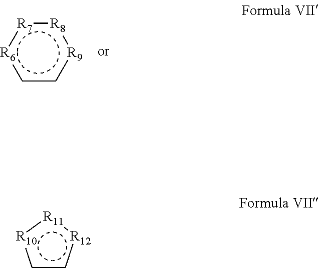

where D′ comprises a chemical probe and/or a biofunctional molecule, where R₆-R₁₂ are each independently C, S, O, or N. In some forms of Formula VII″, one of R₁₀ or R₁₂ is S, the other of R₁₀ or R₁₂ is C, and R₁₁ is C. In some forms, D′ further comprises a linker coupled to the ring of Formula VI and to the chemical probe and/or biofunctional molecule. In some forms, D′ is —R₄—(CH₂)ₙ—Z, where R₄ is:

a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where n is zero or a positive integer; and

where Z is optional and comprises a chemical probe and/or a biofunctional molecule.

In some forms, the compound can have the structure of Formula II:

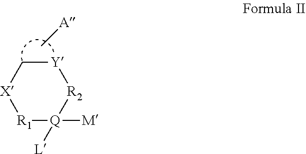

where X′, R₁, R₂, Q, L′, and M′ are as defined above;

where A″ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; and

where Y′ is a nitrogen atom.

In some forms, A″ can be an unsubstituted polyaryl group, a substituted polyaryl group, an unsubstituted polyheteroaryl group, or a substituted polyheteroaryl group. In some forms, A″ can be an unsubstituted polyheteroaryl group or a substituted polyheteroaryl group. In some forms, A″ can be an unsubstituted polyheteroaryl group.

In some forms, A″ can be a substituted polyheteroaryl group.

In some forms, A″ is

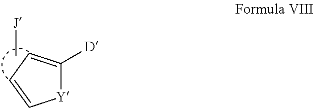

where J′ is

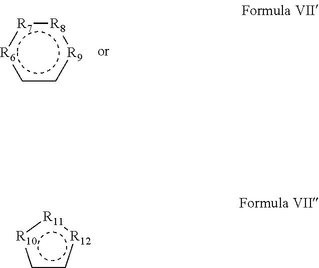

where D′ comprises a chemical probe and/or a biofunctional molecule, where R₆-R₁₂ are each independently C, S, O, or N. In some forms of Formula VII″, one of R₁₀ or R₁₂ is S, the other of R₁₀ or R₁₂ is C, and R₁₁ is C. In some forms, D′ further comprises a linker coupled to the ring of Formula VI and to the chemical probe and/or biofunctional molecule. In some forms, D′ is —R₄—(CH₂)ₙ—Z, where R₄ is:

a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where n is zero or a positive integer; and

where Z is optional and comprises a chemical probe and/or a biofunctional molecule.

In some forms, the compound can have the structure of Formula III:

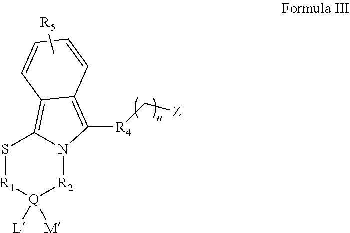

where R₁, R₂, Q, L′, and M′ are as defined above;

where R₄ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where R₅ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group, an unsubstituted carbonyl group, a substituted carbonyl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where n is zero or a positive integer; and

where Z is optional and comprises a chemical probe and/or a biofunctional molecule.

In some forms, the compound can have the structure of Formula III′ or Formula III″:

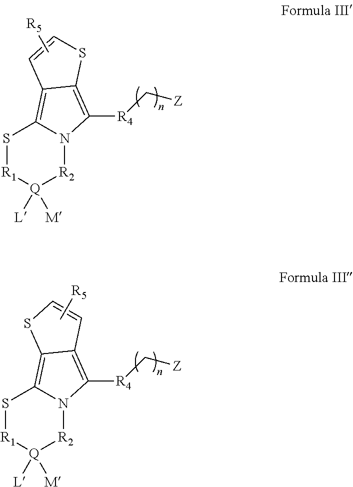

where R₁, R₂, R₄, R₅, Q, L′, M′, n and Z are as defined above.

In a particular form, when R₄ is hydrogen, n can be zero, and Z can be absent. In some forms, R₄ can be an unsubstituted alkenyl group, a substituted alkenyl group, an unsubstituted heteroalkenyl group, or a substituted heteroalkenyl group. In some forms, R₄ can be a substituted alkenyl group or a substituted heteroalkenyl group. In some forms, R₄ can be a substituted alkenyl group. In some forms, R₄ can be an unsubstituted succinimidyl group or a substituted succinimidyl group. In some forms, R₄ can be an unsubstituted succinimidyl group or a substituted succinimidyl group. In some forms, R₄ can be an unsubstituted succinimidyl group. In some forms, R₄ can be a substituted succinimidyl group.

In some forms, Z, if present, can be or contain a luminescence probe. In some forms, the luminescence probe can be an organic dye, a biological fluorophore, or a quantum dot. In some forms, the luminescence probe can be an organic dye. In some forms, the organic dye can be fluorescein, rhodamine, or derivatives thereof. In some forms, the luminescence probe can be a biological fluorophore. In some forms, the luminescence probe can be a quantum dot. In some forms, Z, if present, can be or contain a colorimetric probe. In some forms, Z, if present, can be or contain a biofunctional molecule. In some forms, the biofunctional molecule can be a glycan, a peptide, an oligonucleotide, a protein, or a small molecule drug. In some forms, the functional molecule can be a glycan. In some forms, the functional molecule can be a peptide. In some forms, the functional molecule can be an oligonucleotide. In some forms, the functional molecule can be a protein. In some forms, the functional molecule can be a small molecule drug. In some forms, Z can contain two or more biofunctional molecules. In some forms, Z, if present, can contain a combination of luminescence probe and biofunctional molecule.

In some forms, the compound of Formula I, Formula II, Formula III, Formula III′ and Formula III″ is fluorescent.

In some forms, the compounds of Formula I, Formula II, Formula III, Formula III′ and Formula III″ can be made by performing a reaction between a compound of Formula IV and a compound of Formula V.

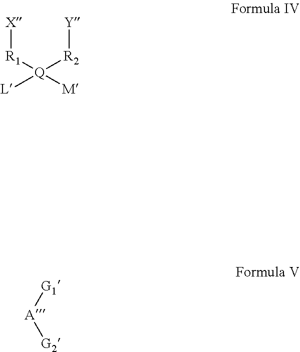

where R₁, R₂, Q, L′, and M′ are as defined above;

where X″ and Y″ are independently a carboxylic acid group, a carboxylate group,

an amino group optionally containing one substituent at the amino nitrogen, where the substituent is a substituted alkyl group, a substituted cycloalkyl group, a substituted heteroalkyl group, a substituted alkenyl group, a substituted heteroalkenyl group, a substituted aryl group, or a substituted heteroaryl group, a hydroxyl group optionally containing one substituent at the hydroxyl oxygen, where the substituent is a substituted alkyl group, a substituted cycloalkyl group, a substituted heteroalkyl group, a substituted alkenyl group, a substituted heteroalkenyl group, a substituted aryl group, or a substituted heteroaryl group, or

a thiol group optionally containing one substituent at the thiol sulfur, where the substituent is a substituted alkyl group, a substituted cycloalkyl group, a substituted heteroalkyl group, a substituted alkenyl group, a substituted heteroalkenyl group, a substituted aryl group, or a substituted heteroaryl group;

where A′″ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; and

where G₁′ and G₂′ are reactive groups.

In some forms, X″ and Y″ can each be independently an amino group optionally containing one substituent at the amino nitrogen, where the substituent is a substituted alkyl group, a substituted cycloalkyl group, a substituted heteroalkyl group, a substituted alkenyl group, a substituted heteroalkenyl group, a substituted aryl group, or a substituted heteroaryl group, or a thiol group optionally containing one substituent at the thiol sulfur, where the substituent is a substituted alkyl group, a substituted cycloalkyl group, a substituted heteroalkyl group, a substituted alkenyl group, a substituted heteroalkenyl group, a substituted aryl group, or a substituted heteroaryl group.

In some forms, X″ and Y″ can each be independently an amine group or a thiol group. In some forms, X″ and Y″ are different and can each be independently an amino group or a thiol group. In some forms, X″ is a thiol group and Y″ is an amino group. In some forms, X″ is a thiol group and Y″ is an amine group.

In some forms, A′″ can be an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group. In some forms, A′″ can be an unsubstituted aryl group, a substituted aryl group, an unsubstituted polyaryl group, or a substituted polyaryl group. In some forms, A′″ can be an unsubstituted aryl group or a substituted aryl group.

In some forms, G₁′ and G₂′ can each be independently an aldehyde group, a cyanate group, a nitrile group, an isonitrile group, a nitro group, a nitroso group, a nitrosooxy group, an acyl group, a carboxylic acid group, or a carboxylate group. In some forms, G₁′ and G₂′ can each be independently an aldehyde group or an acyl group. In some forms, G₁′ and G₂′ are the same and can be aldehyde groups or acyl groups. In some forms, G₁′ and G₂′ are the same and can be aldehyde groups.

In a particular form, the compound of Formula V is ortho-phthalaldehyde (OPA). In another particular form, the compound of Formula V is 2,3-Thiophenedicarboxaldehyde (TDA).

In some forms, the compounds of Formula I, Formula II, Formula III, Formula III′ and Formula III″ can be made by (a) performing a reaction between a compound of Formula IV and a compound of Formula V to form an adduct, where Formula IV and Formula V are as defined above, and (b) performing a reaction between the adduct from step (a) and a reactant to form a second adduct.

In some forms, the reactant can be an unsubstituted maleimide, a substituted maleimide, an unsubstituted alkynyl group, a substituted alkynyl group, or derivatives thereof. In some forms, the reactant can be an unsubstituted maleimide, a substituted maleimide, or derivatives thereof. In some forms, the reactant can be a maleimide derivative. In some forms, the reactant can be an unsubstituted alkynyl group, a substituted alkynyl group, or derivatives thereof. In some forms, the reactant can be a derivatized alkynyl group.

In some forms, the reaction of step (a) can be performed in a buffer solution. In some forms, the reaction of step (b) can be performed in a buffer solution. In some forms, the reactions of step (a) and step (b) can each be performed independently in a buffer solution. In some forms, the reactions of step (a) and step (b) can be performed in the same buffer solution. In some forms, the buffer solution can be acetate buffer, phosphate buffer, HEPES buffer, TEAA buffer, or borate buffer. In some forms, the reaction of step (a) can be performed at a pH of at least about 6, preferably at least about 7, more preferably at least about 7.4. In some forms, the reaction of step (a) can be performed at a pH of at least about 7. In some forms, the reaction of step (a) can be performed at a pH of at least about 7.4. In some forms, the reaction of step (a) can be performed at a pH between about 6 and about 10, between about 6.5 and about 10, between about 6.8 and about 10, between about 7 and about 10, between about 7.4 and about 10, or between about 8 and about 10.

In some forms, the reaction of step (b) can be performed at a pH of at least about 6, preferably at least about 7, more preferably at least about 7.4. In some forms, the reaction of step (b) can be performed at a pH of at least about 7. In some forms, the reaction of step (b) can be performed at a pH of at least about 7.4. In some forms, the reaction of step (b) can be performed at a pH between about 6 and about 10, between about 6.5 and about 10, between about 6.8 and about 10, between about 7 and about 10, between about 7.4 and about 10, or between about 8 and about 10.

In some forms, the reaction of step (a) is performed at a pH different from the reaction of step (b). In some forms, the reaction of step (a) is performed at the same pH as the reaction of step (b). In some forms, the reactions of both step (a) and step (b) are performed at a pH of at least about 7.4. In some forms, the reactions of both step (a) and step (b) are performed at a pH of at least about 8, preferably at least about 8.5.

The progress or completion of the reaction of a given step can be referred to in terms of an amount or percentage of reactant(s) consumed or product produced, for example, at a given time of reaction, after a given time of reaction, by a given time of reaction, and/or for a given time of reaction (all times beginning at the start of the reaction). For example, in some forms, the reaction of step (a) can be performed at a rate where at least 80% of the compound of Formula IV and/or of the compound of Formula V has reacted at about 2.5 hours, preferably at about 2 hours, more preferably at about 1.5 hours. In some forms, the reaction of step (a) can be performed at a rate where at least 80% of the compound of Formula IV has reacted at about 2.5 hours, preferably at about 2 hours, more preferably at about 1.5 hours. In some forms, the reaction of step (a) can be performed at a rate where at least 80% of the compound of Formula V has reacted at about 2.5 hours, preferably at about 2 hours, more preferably at about 1.5 hours.

In some forms, the reaction of step (a) can be performed for a time that results in at least 80% of the compound of Formula IV and/or of the compound of Formula V has reacted. In some forms, the reaction of step (a) can be performed for a time that results in at least 80% of the compound of Formula IV has reacted. In some forms, the reaction of step (a) can be performed for a time that results in at least 80% of the compound of Formula V has reacted.

In some forms, the reaction of step (a) can be performed at a rate where at least 80% of the compound of Formula IV and/or of the compound of Formula V has reacted at about 30 minutes, preferably at about 20 minutes, more preferably at about 10 minutes. In some forms, the reaction of step (a) can be performed at a rate where at least 80% of the compound of Formula IV has reacted at about 30 minutes, preferably at about 20 minutes, more preferably at about 10 minutes. In some forms, the reaction of step (a) can be performed at a rate where at least 80% of the compound of Formula V has reacted at about 30 minutes, preferably at about 20 minutes, more preferably at about 10 minutes.

In some forms, the reaction of step (b) can be performed at a rate where at least 80% of the adduct formed in step (a) and/or the reactant has reacted at about 30 minutes, preferably at about 20 minutes, more preferably at about 10 minutes. In some forms, the reaction of step (b) can be performed at a rate where at least 80% of the adduct formed in step (a) has reacted at about 30 minutes, preferably at about 20 minutes, more preferably at about 10 minutes. In some forms, the reaction of step (b) can be performed at a rate where at least 80% of the reactant has reacted at about 30 minutes, preferably at about 20 minutes, more preferably at about 10 minutes.

In some forms, the reaction of step (a) can be performed at a rate different from that of step (b). In some forms, the reaction of step (a) can be performed at a rate the same as that of step (b).

In some forms, the reaction of step (a) can reach a conversion of at least about 70%, preferably at least about 80%, more preferably at least about 90%. In some forms, the reaction of step (a) can reach a conversion of at least about 80%. In some forms, the reaction of step (a) can reach a conversion of at least about 90%.

In some forms, the reaction of step (b) can reach a conversion of at least about 70%, preferably at least about 80%, more preferably at least about 90%. In some forms, the reaction of step (b) can reach a conversion of at least about 80%. In some forms, the reaction of step (a) can reach a conversion of at least about 90%.

In some forms, the conversion reached by the reaction of step (a) is different from that by the reaction of step (b). In some forms, the conversion reached by the reaction of step (a) is higher than that by the reaction of step (b). In some forms, the conversion reached by the reaction of step (a) is lower than that by the reaction of step (b).

Kits for synthesizing the disclosed compounds are disclosed. The kits contain, in one or more containers, one or more of the disclosed compounds of Formula IV and Formula V, optionally one or more of the disclosed reactants, one or more buffers, instructions for use, and, optionally, one or more carries, and/or an ionic or non-ionic detergent.

Methods of using the disclosed compounds in drug discovery and chemical biology study are also disclosed.

Additional advantages of the disclosed compounds, kits, and methods will be set forth in part in the description which follows, and in part will be understood from the description, or may be learned by practice of the disclosed compounds, kits, and methods. The advantages of the disclosed compounds, kits, and methods will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed compounds, kits, and methods, and together with the description, serve to explain the principles of the disclosed compounds, kits, and methods.

FIG. 1 is a bar graph showing the reaction between Ac-KAAACH-CONH₂ (SEQ ID NO:16) (0.5 mM) and OPA (1 equiv.) under various pH buffer for 15 min.

FIG. 2 is a graph showing the stability results of OPA-cyclized DMAC modified peptide and OPA-cyclized peptide using model peptide Ac-ENPECILDKHVQRVM-CONH₂ (SEQ ID NO:10).

FIG. 3A is a graph showing the flow cytometry analysis of the binding capability of fluorescein modified peptides cKC10′-F, cKC9′-F, and cCK9′-F with Caco2 cells. FIG. 3B is a graph showing the bodings of rhodamine modified cyclo-peptides, cKC10′-R, cKC9′-R, and cCK9′-R with Cacos cells. KC-10′: Ac-KTPSPFDSHC-CONH₂ (SEQ ID NO:25), KC-9′: Ac-KSDSWHYWC-CONH₂ (SEQ ID NO:26), CK-9′: Ac-CPIEDRPMK-CONH₂ (SEQ ID NO:27), F: fluorescein, R: rhodamine, Neg: DMSO. *: P value<0.05, **: P value<0.01, ***: P value<0.001.

FIG. 4A is a graph showing the flow cytometry analysis of the binding capability of fluorescein modified peptides cKC10′-F, cKC9′-F, and cCK9′-F with HT116 cells. FIG. 4B is a graph showing the bodings of rhodamine modified cyclo-peptides, cKC10′-R, cKC9′-R, and cCK9′-R with HT116 cells. KC-10′: Ac-KTPSPFDSHC-CONH₂ (SEQ ID NO:25), KC-9′: Ac-KSDSWHYWC-CONH₂ (SEQ ID NO:26), CK-9′: Ac-CPIEDRPMK-CONH₂ (SEQ ID NO:27), F: fluorescein, R: rhodamine, Neg: DMSO. *: P value<0.05, **: P value<0.01, ***: P value<0.001.

FIG. 5A is a graph showing the flow cytometry analysis of the binding capability of fluorescein modified peptides cKC10′-F, cKC9′-F, and cCK9′-F with A431 cells. FIG. 5B is a graph showing the bodings of rhodamine modified cyclo-peptides, cKC10′-R, cKC9′-R, and cCK9′-R with A431 cells. KC-10′: Ac-KTPSPFDSHC-CONH₂ (SEQ ID NO:25), KC-9′: Ac-KSDSWHYWC-CONH₂ (SEQ ID NO:26), CK-9′: Ac-CPIEDRPMK-CONH₂ (SEQ ID NO:27), F: fluorescein, R: rhodamine, Neg: DMSO. *: P value<0.05, **: P value<0.01, ***: P value<0.001.

FIG. 6A is a bar graph showing the binding of fluorescein modified peptides (cKC10′-F, cKC9′-F, and cCK9′-F) with different cell lines (Caco2, HT116, and A431). FIG. 6B is a bar graph showing the binding of rhodamine modified peptides (cKC10′-R, cKC9′-R, and cCK9′-R) with different cell lines (Caco2, HT116, and A431).

FIG. 7 is a schematic diagram of the OPA-mediated one-pot cyclization and bioconjugation.

### DETAILED DESCRIPTION OF THE INVENTION

The disclosed compounds, kits, and methods may be understood more readily by reference to the following detailed description of particular embodiments and the Examples included therein and to the Figures and their previous and following description.

All methods described herein can be performed in any suitable order unless otherwise indicated herein or otherwise clearly contradicted by context. The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The disclosed compounds and kits, can be used for, can be used in conjunction with, can be used in preparation for, or are products of the disclosed methods. It is understood that when combinations, subsets, interactions, groups, etc. of these compounds and kits are disclosed, while specific reference of each various individual and collective combinations of these materials may not be explicitly disclosed, each is specifically contemplated and described herein. For example, if a compound is disclosed and discussed and a number of modifications that can be made to a number of molecules including the compound are discussed, each and every combination and permutation of the compound and the modifications that are possible are specifically contemplated unless specifically indicated to the contrary.

Further, each of the compounds, kits, components, etc. contemplated and disclosed as above can also be specifically and independently included or excluded from any group, subgroup, list, set, etc. of such materials. These concepts apply to all aspects of this application including, but not limited to, steps in methods of making and using the disclosed compounds, compositions, mixtures, and kits. Thus, if there are a variety of additional steps that can be performed it is understood that each of these additional steps can be performed with any specific embodiment or combination of embodiments of the disclosed methods, and that each such combination is specifically contemplated and should be considered disclosed.

### I. Definitions

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The terms “can,” and “can be,” and related terms are intended to convey that the subject matter involved is optional (that is, the subject matter is present in some forms and is not present in other forms), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present and instances where it does not occur or is not present.

As used herein, the term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear or branched. “Cycloalkyl group” refers to an alkyl group that is cyclic. Preferred alkyl groups have one to 30 carbon atoms, i.e., C₁-C₃₀ alkyl. In some forms, a C₁-C₃₀ alkyl can be a linear C₁-C₃₀ alkyl, a branched C₁-C₃₀ alkyl, or a linear or branched C₁-C₃₀ alkyl. More preferred alkyl groups have one to 20 carbon atoms, i.e., C₁-C₂₀ alkyl. In some forms, a C₁-C₂₀ alkyl can be a linear C₁-C₂₀ alkyl, a branched C₁-C₂₀ alkyl, or a linear or branched C₁-C₂₀ alkyl. Still more preferred alkyl groups have one to 10 carbon atoms, i.e., C₁-C₂₀ alkyl. In some forms, a C₁-C₁₀ alkyl can be a linear C₁-C₁₀ alkyl, a branched C₁-C₁₀ alkyl, or a linear or branched C₁-C₁₀ alkyl. The most preferred alkyl groups have one to 6 carbon atoms, i.e., C₁-C₆ alkyl. In some forms, a C₁-C₆ alkyl can be a linear C₁-C₆ alkyl, a branched C₁-C₆ alkyl, or a linear or branched C₁-C₆ alkyl. Preferred C₁-C₆ alkyl groups have one to four carbons, i.e., C₁-C₄ alkyl. In some forms, a C₁-C₄ alkyl can be a linear C₁-C₄ alkyl, a branched C₁-C₄ alkyl, or a linear or branched C₁-C₄ alkyl. Any C₁-C₃₀ alkyl, C₁-C₂₀ alkyl, C₁-C₁₀ alkyl, C₁-C₆ alkyl, and/or C₁-C₄ alkyl groups can, alternatively, be cyclic. If the alkyl is branched, it is understood that at least four carbons are present. If the alkyl is cyclic, it is understood that at least three carbons are present.

As used herein, the term “heteroalkyl” refers to alkyl groups where one or more carbon atoms are replaced with a heteroatom, such as, O, N, or S. Heteroalkyl groups can be linear or branched. “Cycloheteroalkyl group” refers to a heteroalkyl group that is cyclic. Preferred heteroalkyl groups have one to 30 carbon atoms, i.e., C₁-C₃₀ heteroalkyl. In some forms, a C₁-C₃₀ heteroalkyl can be a linear C₁-C₃₀ heteroalkyl, a branched C₁-C₃₀ heteroalkyl, or a linear or branched C₁-C₃₀ heteroalkyl. More preferred heteroalkyl groups have one to 20 carbon atoms, i.e., C₁-C₂₀ heteroalkyl. In some forms, a C₁-C₂₀ heteroalkyl can be a linear C₁-C₂₀ heteroalkyl, a branched C₁-C₂₀ heteroalkyl, or a linear or branched C₁-C₂₀ heteroalkyl. Still more preferred heteroalkyl groups have one to 10 carbon atoms, i.e., C₁-C₂₀ heteroalkyl. In some forms, a C₁-C₁₀ heteroalkyl can be a linear C₁-C₁₀ heteroalkyl, a branched C₁-C₁₀ heteroalkyl, or a linear or branched C₁-C₁₀ heteroalkyl. The most preferred heteroalkyl groups have one to 6 carbon atoms, i.e., C₁-C₆ heteroalkyl. In some forms, a C₁-C₆ heteroalkyl can be a linear C₁-C₆ heteroalkyl, a branched C₁-C₆ heteroalkyl, or a linear or branched C₁-C₆ heteroalkyl. Preferred C₁-C₆ heteroalkyl groups have one to four carbons, i.e., C₁-C₄ heteroalkyl. In some forms, a C₁-C₄ heteroalkyl can be a linear C₁-C₄ heteroalkyl, a branched C₁-C₄ heteroalkyl, or a linear or branched C₁-C₄ heteroalkyl. If the heteroalkyl is branched, it is understood that at least four carbons are present. If the heteroalkyl is cyclic, it is understood that at least three carbons are present.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic. Preferred alkenyl groups have two to 30 carbon atoms, i.e., C₂-C₃₀ alkenyl. In some forms, a C₂-C₃₀ alkenyl can be a linear C₂-C₃₀ alkenyl, a branched C₂-C₃₀ alkenyl, a cyclic C₂-C₃₀ alkenyl, a linear or branched C₂-C₃₀ alkenyl, a linear or cyclic C₂-C₃₀ alkenyl, a branched or cyclic C₂-C₃₀ alkenyl, or a linear, branched, or cyclic C₂-C₃₀ alkenyl. More preferred alkenyl groups have two to 20 carbon atoms, i.e., C₂-C₂₀ alkenyl. In some forms, a C₂-C₂₀ alkenyl can be a linear C₂-C₂₀ alkenyl, a branched C₂-C₂₀ alkenyl, a cyclic C₂-C₂₀ alkenyl, a linear or branched C₂-C₂₀ alkenyl, a branched or cyclic C₂-C₂₀ alkenyl, or a linear, branched, or cyclic C₂-C₂₀ alkenyl. Still more preferred alkenyl groups have two to 10 carbon atoms, i.e., C₂-C₁₀ alkenyl. In some forms, a C₂-C₁₀ alkenyl can be a linear C₂-C₁₀ alkenyl, a branched C₂-C₁₀ alkenyl, a cyclic C₂-C₁₀ alkenyl, a linear or branched C₂-C₁₀ alkenyl, a branched or cyclic C₂-C₁₀ alkenyl, or a linear, branched, or cyclic C₂-C₂₀ alkenyl. The most preferred alkenyl groups have two to 6 carbon atoms, i.e., C₂-C₆ alkenyl. In some forms, a C₂-C₆ alkenyl can be a linear C₂-C₆ alkenyl, a branched C₂-C₆ alkenyl, a cyclic C₂-C₆ alkenyl, a linear or branched C₂-C₆ alkenyl, a branched or cyclic C₂-C₆ alkenyl, or a linear, branched, or cyclic C₂-C₆ alkenyl. Preferred C₂-C₆ alkenyl groups have two to four carbons, i.e., C₂-C₄ alkenyl. In some forms, a C₂-C₄ alkenyl can be a linear C₂-C₄ alkenyl, a branched C₂-C₄ alkenyl, a cyclic C₂-C₄ alkenyl, a linear or branched C₂-C₄ alkenyl, a branched or cyclic C₂-C₄ alkenyl, or a linear, branched, or cyclic C₂-C₄ alkenyl. If the alkenyl is branched, it is understood that at least four carbons are present. If the alkenyl is cyclic, it is understood that at least three carbons are present.

As used herein, the term “heteroalkenyl” refers to alkenyl groups in which one or more doubly bonded carbon atoms are replaced by a heteroatom. Heteroalkenyl groups can be linear, branched, or cyclic. Preferred heteroalkenyl groups have two to carbon atoms, i.e., C₂-C₃₀ heteroalkenyl. In some forms, a C₂-C₃₀ heteroalkenyl can be a linear C₂-C₃₀ heteroalkenyl, a branched C₂-C₃₀ heteroalkenyl, a cyclic C₂-C₃₀ heteroalkenyl, a linear or branched C₂-C₃₀ heteroalkenyl, a linear or cyclic C₂-C₃₀ heteroalkenyl, a branched or cyclic C₂-C₃₀ heteroalkenyl, or a linear, branched, or cyclic C₂-C₃₀ heteroalkenyl. More preferred heteroalkenyl groups have two to 20 carbon atoms, i.e., C₂-C₂₀ heteroalkenyl. In some forms, a C₂-C₂₀ heteroalkenyl can be a linear C₂-C₂₀ heteroalkenyl, a branched C₂-C₂₀ heteroalkenyl, a cyclic C₂-C₂₀ heteroalkenyl, a linear or branched C₂-C₂₀ heteroalkenyl, a branched or cyclic C₂-C₂₀ heteroalkenyl, or a linear, branched, or cyclic C₂-C₂₀ heteroalkenyl. Still more preferred heteroalkenyl groups have two to 10 carbon atoms, i.e., C₂-C₁₀ heteroalkenyl. In some forms, a C₂-C₁₀ heteroalkenyl can be a linear C₂-C₁₀ heteroalkenyl, a branched C₂-C₁₀ heteroalkenyl, a cyclic C₂-C₁₀ heteroalkenyl, a linear or branched C₂-C₁₀ heteroalkenyl, a branched or cyclic C₂-C₁₀ heteroalkenyl, or a linear, branched, or cyclic C₂-C₂₀ heteroalkenyl. The most preferred heteroalkenyl groups have two to 6 carbon atoms, i.e., C₂-C₆ heteroalkenyl. In some forms, a C₂-C₆ heteroalkenyl can be a linear C₂-C₆ heteroalkenyl, a branched C₂-C₆ heteroalkenyl, a cyclic C₂-C₆ heteroalkenyl, a linear or branched C₂-C₆ heteroalkenyl, a branched or cyclic C₂-C₆ heteroalkenyl, or a linear, branched, or cyclic C₂-C₆ heteroalkenyl. Preferred C₂-C₆ heteroalkenyl groups have two to four carbons, i.e., C₂-C₄ heteroalkenyl. In some forms, a C₂-C₄ heteroalkenyl can be a linear C₂-C₄ heteroalkenyl, a branched C₂-C₄ heteroalkenyl, a cyclic C₂-C₄ heteroalkenyl, a linear or branched C₂-C₄ heteroalkenyl, a branched or cyclic C₂-C₄ heteroalkenyl, or a linear, branched, or cyclic C₂-C₄ heteroalkenyl. If the heteroalkenyl is branched, it is understood that at least four carbons are present. If heteroalkenyl is cyclic, it is understood that at least three carbons are present.

As used herein, the term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic. Preferred alkynyl groups have two to 30 carbon atoms, i.e., C₂-C₃₀ alkynyl. In some forms, a C₂-C₃₀ alkynyl can be a linear C₂-C₃₀ alkynyl, a branched C₂-C₃₀ alkynyl, a cyclic C₂-C₃₀ alkynyl, a linear or branched C₂-C₃₀ alkynyl, a linear or cyclic C₂-C₃₀ alkynyl, a branched or cyclic C₂-C₃₀ alkynyl, or a linear, branched, or cyclic C₂-C₃₀ alkynyl. More preferred alkynyl groups have two to 20 carbon atoms, i.e., C₂-C₂₀ alkynyl. In some forms, a C₂-C₂₀ alkynyl can be a linear C₂-C₂₀ alkynyl, a branched C₂-C₂₀ alkynyl, a cyclic C₂-C₂₀ alkynyl, a linear or branched C₂-C₂₀ alkynyl, a branched or cyclic C₂-C₂₀ alkynyl, or a linear, branched, or cyclic C₂-C₂₀ alkynyl. Still more preferred alkynyl groups have two to 10 carbon atoms, i.e., C₂-C₁₀ alkynyl. In some forms, a C₂-C₁₀ alkynyl can be a linear C₂-C₁₀ alkynyl, a branched C₂-C₁₀ alkynyl, a cyclic C₂-C₁₀ alkynyl, a linear or branched C₂-C₁₀ alkynyl, a branched or cyclic C₂-C₁₀ alkynyl, or a linear, branched, or cyclic C₂-C₂₀ alkynyl. The most preferred alkynyl groups have two to 6 carbon atoms, i.e., C₂-C₆ alkynyl. In some forms, a C₂-C₆ alkynyl can be a linear C₂-C₆ alkynyl, a branched C₂-C₆ alkynyl, a cyclic C₂-C₆ alkynyl, a linear or branched C₂-C₆ alkynyl, a branched or cyclic C₂-C₆ alkynyl, or a linear, branched, or cyclic C₂-C₆ alkynyl. Preferred C₂-C₆ alkynyl groups have two to four carbons, i.e., C₂-C₄ alkynyl. In some forms, a C₂-C₄ alkynyl can be a linear C₂-C₄ alkynyl, a branched C₂-C₄ alkynyl, a cyclic C₂-C₄ alkynyl, a linear or branched C₂-C₄ alkynyl, a branched or cyclic C₂-C₄ alkynyl, or a linear, branched, or cyclic C₂-C₄ alkynyl. If the alkynyl is branched, it is understood that at least four carbons are present. If alkynyl is cyclic, it is understood that at least three carbons are present.

As used herein, the term “heteroalkynyl” refers to alkynyl groups in which one or more triply bonded carbon atoms are replaced by a heteroatom. Heteroalkynyl groups can be linear, branched, or cyclic. Preferred heteroalkynyl groups have two to carbon atoms, i.e., C₂-C₃₀ heteroalkynyl. In some forms, a C₂-C₃₀ heteroalkynyl can be a linear C₂-C₃₀ heteroalkynyl, a branched C₂-C₃₀ heteroalkynyl, a cyclic C₂-C₃₀ heteroalkynyl, a linear or branched C₂-C₃₀ heteroalkynyl, a linear or cyclic C₂-C₃₀ heteroalkynyl, a branched or cyclic C₂-C₃₀ heteroalkynyl, or a linear, branched, or cyclic C₂-C₃₀ heteroalkynyl. More preferred heteroalkynyl groups have two to 20 carbon atoms, i.e., C₂-C₂₀ heteroalkynyl. In some forms, a C₂-C₂₀ heteroalkynyl can be a linear C₂-C₂₀ heteroalkynyl, a branched C₂-C₂₀ heteroalkynyl, a cyclic C₂-C₂₀ heteroalkynyl, a linear or branched C₂-C₂₀ heteroalkynyl, a branched or cyclic C₂-C₂₀ heteroalkynyl, or a linear, branched, or cyclic C₂-C₂₀ heteroalkynyl. Still more preferred heteroalkynyl groups have two to 10 carbon atoms, i.e., C₂-C₁₀ heteroalkynyl. In some forms, a C₂-C₁₀ heteroalkynyl can be a linear C₂-C₁₀ heteroalkynyl, a branched C₂-C₁₀ heteroalkynyl, a cyclic C₂-C₁₀ heteroalkynyl, a linear or branched C₂-C₁₀ heteroalkynyl, a branched or cyclic C₂-C₁₀ heteroalkynyl, or a linear, branched, or cyclic C₂-C₂₀ heteroalkynyl. The most preferred heteroalkynyl groups have two to 6 carbon atoms, i.e., C₂-C₆ heteroalkynyl. In some forms, a C₂-C₆ heteroalkynyl can be a linear C₂-C₆ heteroalkynyl, a branched C₂-C₆ heteroalkynyl, a cyclic C₂-C₆ heteroalkynyl, a linear or branched C₂-C₆ heteroalkynyl, a branched or cyclic C₂-C₆ heteroalkynyl, or a linear, branched, or cyclic C₂-C₆ heteroalkynyl. Preferred C₂-C₆ heteroalkynyl groups have two to four carbons, i.e., C₂-C₄ heteroalkynyl. In some forms, a C₂-C₄ heteroalkynyl can be a linear C₂-C₄ heteroalkynyl, a branched C₂-C₄ heteroalkynyl, a cyclic C₂-C₄ heteroalkynyl, a linear or branched C₂-C₄ heteroalkynyl, a branched or cyclic C₂-C₄ heteroalkynyl, or a linear, branched, or cyclic C₂-C₄ heteroalkynyl. If the heteroalkynyl is branched, it is understood that at least four carbons are present. If heteroalkynyl is cyclic, it is understood that at least three carbons are present.

As used herein, the term “aryl” refers to univalent groups derived from arenes by removal of a hydrogen atom from a ring atom. Arenes are monocyclic and polycyclic aromatic hydrocarbons. In polycyclic aryl groups, the rings can be attached together in a pendant manner or can be fused. Preferred aryl groups have six to 50 carbon atoms, i.e., C₆-C₅₀ aryl. In some forms, a C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl. More preferred aryl groups have six to 30 carbon atoms, i.e., C₆-C₃₀ aryl. In some forms, a C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Even more preferred aryl groups have six to 20 carbon atoms, i.e., C₆-C₂₀ aryl. In some forms, a C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl. The most preferred aryl groups have six to twelve carbon atoms, i.e., C₆-C₁₂ aryl. In some forms, a C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Preferred C₆-C₁₂ aryl groups have six to eleven carbon atoms, i.e., C₆-C₁₁ aryl. In some forms, a C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. More preferred C₆-C₁₂ aryl groups have six to nine carbon atoms, i.e., C₆-C₉ aryl. In some forms, a C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. The most preferred C₆-C₁₂ aryl groups have six carbon atoms, i.e., C₆ aryl. In some forms, a C₆ aryl can be a branched C₆ aryl or a monocyclic C₆ aryl.

As used herein, the term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). In polycyclic heteroaryl groups, the rings can be attached together in a pendant manner or can be fused. Preferred heteroaryl groups have three to 50 carbon atoms, i.e., C₃-C₅₀ heteroaryl. In some forms, a C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. More preferred heteroaryl groups have six to 30 carbon atoms, i.e., C₆-C₃₀ heteroaryl. In some forms, a C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₀ heteroaryl. Even more preferred heteroaryl groups have six to 20 carbon atoms, i.e., C₆-C₂₀ heteroaryl. In some forms, a C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂₀ heteroaryl. The most preferred heteroaryl groups have six to twelve carbon atoms, i.e., C₆-C₁₂ heteroaryl. In some forms, a C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Preferred C₆-C₁₂ heteroaryl groups have six to eleven carbon atoms, i.e., C₆-C₁₁ heteroaryl. In some forms, a C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. More preferred C₆-C₁₂ heteroaryl groups have six to nine carbon atoms, i.e., C₆-C₉ heteroaryl. In some forms, a C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. The most preferred C₆-C₁₂ heteroaryl groups have six carbon atoms, i.e., C₆ heteroaryl. In some forms, a C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

As used herein, the term “hydroxamate” refers to —C(═O)NH—OH, where the hydrogen atoms can be substituted with substituents.

As used herein, the term “oligomer” refers to multimers of subunits (e.g., monomers, building blocks) having a small or moderate number of monomer resides. Notable examples of oligomers are peptides, oligonucleotides, and oligomers of synthetic monomers. A multimer is any chain of two or more monomer residues. An oligomer is any multimer having from two to 100 monomer residues. Generally, an oligomer can have from two to 100 monomer residues, preferably five to 50 monomer residues, most preferably from five to 20 monomer residues. A polymer is any multimer having 20 or more monomer residues. Generally, a polymer can have 50 or more monomer residues, 75 or more monomer residues, or 100 or more monomer residues. Thus, the terms multimers, oligomer, and polymer overlap, but oligomers and polymers have different length domains.

As used herein, the term “monomer” refers to a unit that is or can be the building block of a multimers, oligomer, polymer, etc. For example, amino acids are the normal building blocks (i.e., monomers) of peptides and proteins. Nucleotides are the normal building blocks (i.e., monomers) of oligonucleotides and polynucleotides. Synthetic monomer subunits (e.g., ethylene glycol subunit, acrylamide subunit, vinyl subunit, etc.) are the building blocks of synthetic oligomers and polymers (e.g., polyethylene glycol, polyacrylamide, polyvinyl, etc.).

As used herein, the term “residue” refers to the part of a monomer subunit that remains or is present in a multimers, oligomer, or polymer in which the monomer residue resides.

As used herein, the term “synthetic material” refers to a non-oligomer, non-polymer component.

As used herein, the term “small molecule drug” refers to an organic compound that can regulate a biological process, with a molecular weight equals or less than 900 daltons.

As used herein, the term “substituted,” means that the chemical group or moiety contains one or more substituents replacing the hydrogen atoms in the chemical group or moiety. The substituents include, but are not limited to:

a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{T1′})(OR^{T2′}), —OP(═O)(OR^{T1′})(OR^{T2′}), —BR^{T1′}(OR^{T2′}), —B(OR^{T1′})(OR^{T2′}), or G′R^{T1′} in which -T′ is —O—, —S—, —NR^{T2′}—, —C(═O)—, —S(═O)—, —SO₂—, —C(═O)O—, —C(═O)NR^{T2′}—, —OC(═O)—, —NR^{T2′}C(═O)—, —OC(═O)O—, —OC(═O)NR^{T2′}—, —NR^{T2′}C(═O)O—, —NR^{T2′}C(═O)NR^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′}), —NR^{T2′}C(═NR^{T3′})—, —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

As used herein, the term “derivative” refers to a compound which is formed from a parent compound by chemical reaction(s).

As used herein, the term “Oligonucleotide” refers to short nucleic acid (i.e., DNA and RNA) molecules. They contain less than 100 nucleotides. Preferably, they contain less than 50 nucleotides. More preferably, they contain 25 or less nucleotides. Most preferably, they contain 13-25 nucleotides.

As used herein, the term “Luminescence” refers to emission of light by a substance not resulting from heat. It can be caused by chemical reactions, electrical energy, subatomic motions or stress on a crystal, which all are ultimately caused by spontaneous emission. It can refer to chemiluminescence, i.e., the emission of light as a result of a chemical reaction. It can also refer to photoluminescence, i.e., the emission of light as a result of absorption of photons. The photoluminescence can include fluorescence and phosphorescence.

As used herein, the terms “carrier” or “carriers” refer to all components present in a formulation other than the active ingredient or ingredients. They can include but are not limited to diluents, binders, lubricants, desintegrators, fillers, plasticizers, pigments, colorants, stabilizing agents, and glidants.

As used herein, the term “conversion” refers to the ratio of the amount of product to the amount of a reactant.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

Numerical ranges disclosed in the present application of any type, disclose individually each possible number that such a range could reasonably encompass, as well as any sub-ranges and combinations of sub-ranges encompassed therein.

### II. Compounds

Disclosed herein are cyclic compounds. In particular, the disclosed compounds have a structures of Formula I or salts thereof.

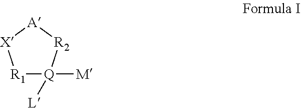

where A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; where X′ is —NR₃, an oxygen atom, or a sulfur atom, where R₃ is a hydrogen, a substituted alkyl group, a substituted cycloalkyl group, a substituted heteroalkyl group, a substituted alkenyl group, a substituted heteroalkenyl group, a substituted aryl group, or a substituted heteroaryl group;

where R₁ and R₂ are independently absent, an unsubstituted alkyl group, a substituted alkyl group, an unsubstituted heteroalkyl group, or a substituted heteroalkyl group;

where Q is a compound of interest; and

where L′ and M′ are independently absent or a compound of interest.

In some forms, X′ is a sulfur atom. In form forms, Q, L′, and M′, if present, are the same type of molecule (that is, the same type of molecule as each other), such as the same type of oligomer. In some forms, Q can be an oligomer or a synthetic material. In some forms, Q can be an oligomer of synthetic monomer residues. In some forms, L′ and M′ are one or more monomer residues or a synthetic material. In some forms, L′ and M′ are one or more monomer residues. In some forms, L′ and M′ are a synthetic material.

In some forms, the monomer residues can each be independently amino acid residues or nucleotide residues. In some forms, the monomer residues can be amino acid residues. In some forms, the monomer residues can be nucleotide residues. In some forms, L′ and M′ can each be independently one or more amino acid residues.

In some forms, Q can be a peptide or an oligonucleotide. In some forms, Q can be a peptide. In some forms, Q can be a linear peptide, a cyclic peptide, or a branched peptide. In some forms, Q can be a linear peptide. In some forms, Q can be a cyclic peptide. In some forms, Q can be a branched peptide. In some forms, Q can be an oligonucleotide. In some forms, Q can be an unprotected peptide. In some forms, Q can be a protected peptide. In some forms, Q can be a polysaccharide. In some forms, Q can be monosaccharides.

In some forms, A′ is

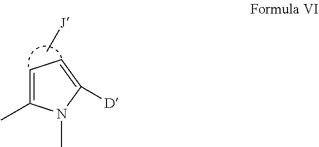

where J′ is

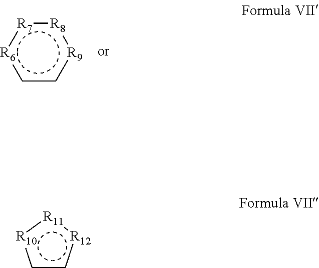

where D′ comprises a chemical probe and/or a biofunctional molecule, where R₆-R₁₂ are each independently C, S, O, or N. In some forms of Formula VII′, each of R₆-R₉ is C. In some forms of Formula VII″, each of R₁₀-R₁₂ is C.

In some forms of Formula VII′, one of R₆-R₉ is S, O, or N and the other of R₆-R₉ are C. In some forms of Formula VII′, two of R₆-R₉ are independently S, O, or N and the other of R₆-R₉ are C. In some forms of Formula VII′, three of R₆-R₉ are independently S, O, or N and the other of R₆-R₉ is C. In some forms of Formula VII′, four of R₆-R₉ are independently S, O, or N. In some forms of Formula VII′, one of R₆-R₉ is S and the other of R₆-R₉ are C. In some forms of Formula VII′, two of R₆-R₉ are S and the other of R₆-R₉ are C. In some forms of Formula VII′, three of R₆-R₉ are S and the other of R₆-R₉ is C. In some forms of Formula VII′, four of R₆-R₉ are S. In some forms of Formula VII′, one of R₆-R₉ is O and the other of R₆-R₉ are C. In some forms of Formula VII′, two of R₆-R₉ are O and the other of R₆-R₉ are C. In some forms of Formula VII′, three of R₆-R₉ are O and the other of R₆-R₉ is C. In some forms of Formula VII′, four of R₆-R₉ are O. In some forms of Formula VII′, one of R₆-R₉ is N and the other of R₆-R₉ are C. In some forms of Formula VII′, two of R₆-R₉ are N and the other of R₆-R₉ are C. In some forms of Formula VII′, three of R₆-R₉ are N and the other of R₆-R₉ is C. In some forms of Formula VII′, four of R₆-R₉ are N.

In some forms of Formula VII″, one of R₁₀-R₁₂ is S, O, or N and the other of R₁₀-R₁₂ are C. In some forms of Formula VII″, two of R₁₀-R₁₂ are independently S, O, or N and the other of R₁₀-R₁₂ is C. In some forms of Formula VII″, three of R₁₀-R₁₂ are independently S, O, or N. In some forms of Formula VII″, one of R₁₀-R₁₂ is S and the other of R₁₀-R₁₂ are C. In some forms of Formula VII″, two of R₁₀-R₁₂ are S and the other of R₁₀-R₁₂ is C. In some forms of Formula VII″, three of R₁₀-R₁₂ are S. In some forms of Formula VII″, one of R₁₀-R₁₂ is O and the other of R₁₀-R₁₂ are C. In some forms of Formula VII″, two of R₁₀-R₁₂ are O and the other of R₁₀-R₁₂ is C. In some forms of Formula VII″, three of R₁₀-R₁₂ are O. In some forms of Formula VII″, one of R₁₀-R₁₂ is N and the other of R₁₀-R₁₂ are C. In some forms of Formula VII″, two of R₁₀-R₁₂ are N and the other of R₁₀-R₁₂ is C. In some forms of Formula VII″, three of R₁₀-R₁₂ are N. In some forms of Formula VII″, one of R₁₀ or R₁₂ is S, O, or N, the other of R₁₀ or R₁₂ is C, and Ru is C. In some forms of Formula VII″, one of R₁₀ or R₁₂ is S, the other of R₁₀ or R₁₂ is C, and Ru is C. In some forms of Formula VII″, one of R₁₀ or R₁₂ is O, the other of R₁₀ or R₁₂ is C, and Ru is C. In some forms of Formula VII″, one of R₁₀ or R₁₂ is N, the other of R₁₀ or R₁₂ is C, and Ru is C.

In some forms, D′ further comprises a linker coupled to the ring of Formula VI and to the chemical probe and/or biofunctional molecule. In some forms, D′ is —R₄—(CH₂)ₙ—Z, where R₄ is:

a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where n is zero or a positive integer; and

where Z is optional and comprises a chemical probe and/or a biofunctional molecule.

In some forms, the compound can have the structure of Formula II:

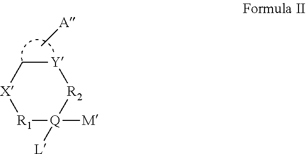

where X′, R₁, R₂, Q, L′, and M′ are as defined above;

where A″ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; and where Y′ is a nitrogen atom.

In some forms, A″ can be an unsubstituted polyaryl group, a substituted polyaryl group, an unsubstituted polyheteroaryl group, or a substituted polyheteroaryl group. In some forms, A″ can be an unsubstituted polyheteroaryl group or a substituted polyheteroaryl group. In some forms, A″ can be an unsubstituted polyheteroaryl group. In some forms, A″ can be a substituted polyheteroaryl group.

In some forms, A″ is

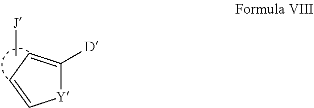

where J′ is

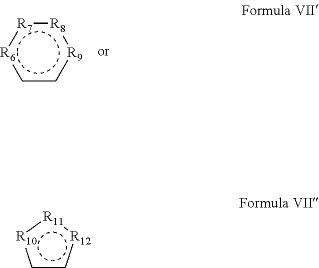

where D′ comprises a chemical probe and/or a biofunctional molecule, where R₆-R₁₂ are each independently C, S, O, or N. In some forms of Formula VII′, each of R₆-R₉ is C. In some forms of Formula VII″, each of R₁₀-R₁₂ is C.

In some forms of Formula VII′, one of R₆-R₉ is S, O, or N and the other of R₆-R₉ are C. In some forms of Formula VII′, two of R₆-R₉ are independently S, O, or N and the other of R₆-R₉ are C. In some forms of Formula VII′, three of R₆-R₉ are independently S, O, or N and the other of R₆-R₉ is C. In some forms of Formula VII′, four of R₆-R₉ are independently S, O, or N. In some forms of Formula VII′, one of R₆-R₉ is S and the other of R₆-R₉ are C. In some forms of Formula VII′, two of R₆-R₉ are S and the other of R₆-R₉ are C. In some forms of Formula VII′, three of R₆-R₉ are S and the other of R₆-R₉ is C. In some forms of Formula VII′, four of R₆-R₉ are S. In some forms of Formula VII′, one of R₆-R₉ is O and the other of R₆-R₉ are C. In some forms of Formula VII′, two of R₆-R₉ are O and the other of R₆-R₉ are C. In some forms of Formula VII′, three of R₆-R₉ are O and the other of R₆-R₉ is C. In some forms of Formula VII′, four of R₆-R₉ are O. In some forms of Formula VII′, one of R₆-R₉ is N and the other of R₆-R₉ are C. In some forms of Formula VII′, two of R₆-R₉ are N and the other of R₆-R₉ are C. In some forms of Formula VII′, three of R₆-R₉ are N and the other of R₆-R₉ is C. In some forms of Formula VII′, four of R₆-R₉ are N.

In some forms of Formula VII″, one of R₁₀-R₁₂ is S, O, or N and the other of R₁₀-R₁₂ are C. In some forms of Formula VII″, two of R₁₀-R₁₂ are independently S, O, or N and the other of R₁₀-R₁₂ is C. In some forms of Formula VII″, three of R₁₀-R₁₂ are independently S, O, or N. In some forms of Formula VII″, one of R₁₀-R₁₂ is S and the other of R₁₀-R₁₂ are C. In some forms of Formula VII″, two of R₁₀-R₁₂ are S and the other of R₁₀-R₁₂ is C. In some forms of Formula VII″, three of R₁₀-R₁₂ are S. In some forms of Formula VII″, one of R₁₀-R₁₂ is O and the other of R₁₀-R₁₂ are C. In some forms of Formula VII″, two of R₁₀-R₁₂ are O and the other of R₁₀-R₁₂ is C. In some forms of Formula VII″, three of R₁₀-R₁₂ are O. In some forms of Formula VII″, one of R₁₀-R₁₂ is N and the other of R₁₀-R₁₂ are C. In some forms of Formula VII″, two of R₁₀-R₁₂ are N and the other of R₁₀-R₁₂ is C. In some forms of Formula VII″, three of R₁₀-R₁₂ are N. In some forms of Formula VII″, one of R₁₀ or R₁₂ is S, O, or N, the other of R₁₀ or R₁₂ is C, and Ru is C. In some forms of Formula VII″, one of R₁₀ or R₁₂ is S, the other of R₁₀ or R₁₂ is C, and Ru is C. In some forms of Formula VII″, one of R₁₀ or R₁₂ is O, the other of R₁₀ or R₁₂ is C, and Ru is C. In some forms of Formula VII″, one of R₁₀ or R₁₂ is N, the other of R₁₀ or R₁₂ is C, and Ru is C.

In some forms, D′ further comprises a linker coupled to the ring of Formula VI and to the chemical probe and/or biofunctional molecule. In some forms, D′ is —R₄—(CH₂)ₙ—Z, where R₄ is:

a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where n is zero or a positive integer; and

where Z is optional and comprises a chemical probe and/or a biofunctional molecule.

In some forms, the compound can have the structure of Formula III:

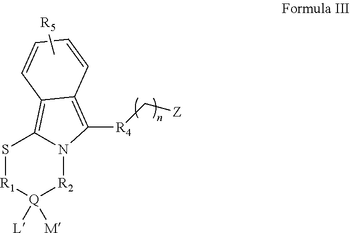

where R₁, R₂, Q, L′, and M′ are as defined above;

where R₄ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where R₅ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group, an unsubstituted carbonyl group, a substituted carbonyl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

where n is zero or a positive integer; and where Z is optional and comprises a chemical probe and/or a biofunctional molecule.

In some forms, the compound can have the structure of Formula III′ or Formula III″:

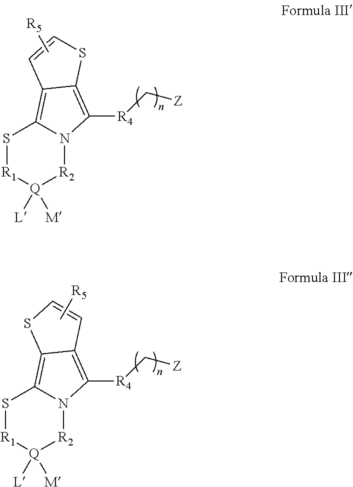

where R₁, R₂, R₄, R₅, Q, L′, M′, n and Z are as defined above.

In a particular form, when R₄ is hydrogen, n can be zero, and Z can be absent. In some forms, R₄ can be an unsubstituted alkenyl group, a substituted alkenyl group, an unsubstituted heteroalkenyl group, or a substituted heteroalkenyl group. In some forms, R₄ can be a substituted alkenyl group or a substituted heteroalkenyl group. In some forms, R₄ can be a substituted alkenyl group. In some forms, R₄ can be an unsubstituted succinimidyl group or a substituted succinimidyl group. In some forms, R₄ can be an unsubstituted succinimidyl group or a substituted succinimidyl group. In some forms, R₄ can be an unsubstituted succinimidyl group. In some forms, R₄ can be a substituted succinimidyl group.

In some forms, Z, if present, can be or contain a luminescence probe. In some forms, the luminescence probe can be an organic dye, a biological fluorophore, or a quantum dot. In some forms, the luminescence probe can be an organic dye. In some forms, the organic dye can be fluorescein, rhodamine, or derivatives thereof. In some forms, the luminescence probe can be a biological fluorophore. In some forms, the luminescence probe can be a quantum dot. Exemplary luminescence probes include, but are not limited to, fluorescein, rhodamine, resorufin, Tokyo Green, coumarin, luciferin, and derivatives thereof.

In some forms, Z, if present, can be or contain a colorimetric probe. Exemplary colorimetric probes include p-nitrophenol, p-thio-nitrobenzoic acid, and derivatives thereof. In some forms, Z, if present, can be or contain a biofunctional molecule. In some forms, the biofunctional molecule can be a glycan, a peptide, an oligonucleotide, a protein, or a small molecule drug. In some forms, the functional molecule can be a glycan. In some forms, the functional molecule can be a peptide. In some forms, the functional molecule can be a protein. In some forms, the functional molecule can be a small molecule drug. In some forms, Z can contain two or more biofunctional molecules. In some forms, Z, if present, can contain a combination of luminescence probe and biofunctional molecule.

In some forms, the compound of Formula I, Formula II, Formula III, Formula III′ and Formula III″ is fluorescent.

Independently in some forms of the disclosed compounds, each R₁, R₂, R₃, R₄, and R₅ can independently be hydrogen or a substituted or unsubstituted C₁-C₃₀ alkyl, linear C₁-C₃₀ alkyl, branched C₁-C₃₀ alkyl, C₁-C₂₀ alkyl, linear C₁-C₂₀ alkyl, branched C₁-C₂₀ alkyl, C₁-C₁₀ alkyl, linear C₁-C₁₀ alkyl, branched C₁-C₁₀ alkyl, C₁-C₆ alkyl, linear C₁-C₆ alkyl, branched C₁-C₆ alkyl, C₁-C₄ alkyl, linear C₁-C₄ alkyl, branched C₁-C₄ alkyl, C₁-C₃₀ heteroalkyl, linear C₁-C₃₀ heteroalkyl, branched C₁-C₃₀ heteroalkyl, C₁-C₂₀ heteroalkyl, linear C₁-C₂₀ heteroalkyl, branched C₁-C₂₀ heteroalkyl, C₁-C₁₀ heteroalkyl, linear C₁-C₁₀ heteroalkyl, branched C₁-C₁₀ heteroalkyl, C₁-C₆ heteroalkyl, linear C₁-C₆ heteroalkyl, branched C₁-C₆ heteroalkyl, C₁-C₄ heteroalkyl, linear C₁-C₄ heteroalkyl, branched C₁-C₄ heteroalkyl, C₂-C₃₀ alkenyl, linear C₂-C₃₀ alkenyl, branched C₂-C₃₀ alkenyl, cyclic C₂-C₃₀ alkenyl, C₂-C₂₀ alkenyl, linear C₂-C₂₀ alkenyl, branched C₂-C₂₀ alkenyl, cyclic C₂-C₂₀ alkenyl, C₂-C₁₀ alkenyl, linear C₂-C₁₀ alkenyl, branched C₂-C₁₀ alkenyl, cyclic C₂-C₁₀ alkenyl, C₂-C₆ alkenyl, linear C₂-C₆ alkenyl, branched C₂-C₆ alkenyl, cyclic C₂-C₆ alkenyl, C₂-C₄ alkenyl, linear C₂-C₄ alkenyl, branched C₂-C₄ alkenyl, cyclic C₂-C₄ alkenyl, C₂-C₃₀ heteroalkenyl, linear C₂-C₃₀ heteroalkenyl, branched C₂-C₃₀ heteroalkenyl, cyclic C₂-C₃₀ heteroalkenyl, C₂-C₂₀ heteroalkenyl, linear C₂-C₂₀ heteroalkenyl, branched C₂-C₂₀ heteroalkenyl, cyclic C₂-C₂₀ heteroalkenyl, C₂-C₁₀ heteroalkenyl, linear C₂-C₁₀ heteroalkenyl, branched C₂-C₁₀ heteroalkenyl, cyclic C₂-C₁₀ heteroalkenyl, C₂-C₆ heteroalkenyl, linear C₂-C₆ heteroalkenyl, branched C₂-C₆ heteroalkenyl, cyclic C₂-C₆ heteroalkenyl, C₂-C₄ heteroalkenyl, linear C₂-C₄ heteroalkenyl, branched C₂-C₄ heteroalkenyl, cyclic C₂-C₄ heteroalkenyl, C₂-C₃₀ alkynyl, linear C₂-C₃₀ alkynyl, branched C₂-C₃₀ alkynyl, cyclic C₂-C₃₀ alkynyl, C₂-C₂₀ alkynyl, linear C₂-C₂₀ alkynyl, branched C₂-C₂₀ alkynyl, cyclic C₂-C₂₀ alkynyl, C₂-C₁₀ alkynyl, linear C₂-C₁₀ alkynyl, branched C₂-C₁₀ alkynyl, cyclic C₂-C₁₀ alkynyl, C₂-C₆ alkynyl, linear C₂-C₆ alkynyl, branched C₂-C₆ alkynyl, cyclic C₂-C₆ alkynyl, C₂-C₄ alkynyl, linear C₂-C₄ alkynyl, branched C₂-C₄ alkynyl, cyclic C₂-C₄ alkynyl, C₂-C₃₀ heteroalkynyl, linear C₂-C₃₀ heteroalkynyl, branched C₂-C₃₀ heteroalkynyl, cyclic C₂-C₃₀ heteroalkynyl, C₂-C₂₀ heteroalkynyl, linear C₂-C₂₀ heteroalkynyl, branched C₂-C₂₀ heteroalkynyl, cyclic C₂-C₂₀ heteroalkynyl, C₂-C₁₀ heteroalkynyl, linear C₂-C₁₀ heteroalkynyl, branched C₂-C₁₀ heteroalkynyl, cyclic C₂-C₁₀ heteroalkynyl, C₂-C₆ heteroalkynyl, linear C₂-C₆ heteroalkynyl, branched C₂-C₆ heteroalkynyl, cyclic C₂-C₆ heteroalkynyl, C₂-C₄ heteroalkynyl, linear C₂-C₄ heteroalkynyl, branched C₂-C₄ heteroalkynyl, cyclic C₂-C₄ heteroalkynyl, C₆-C₅₀ aryl, branched C₆-C₅₀ aryl, monocyclic C₆-C₅₀ aryl, polycyclic C₆-C₅₀ aryl, branched polycyclic C₆-C₅₀ aryl, fused polycyclic C₆-C₅₀ aryl, branched fused polycyclic C₆-C₅₀ aryl, C₆-C₃₀ aryl, branched C₆-C₃₀ aryl, monocyclic C₆-C₃₀ aryl, polycyclic C₆-C₃₀ aryl, branched polycyclic C₆-C₃₀ aryl, fused polycyclic C₆-C₃₀ aryl, branched fused polycyclic C₆-C₃₀ aryl, C₆-C₂₀ aryl, branched C₆-C₂₀ aryl, monocyclic C₆-C₂₀ aryl, polycyclic C₆-C₂₀ aryl, branched polycyclic C₆-C₂₀ aryl, fused polycyclic C₆-C₂₀ aryl, or branched fused polycyclic C₆-C₂₀ aryl, C₆-C₁₂ aryl, branched C₆-C₁₂ aryl, monocyclic C₆-C₁₂ aryl, polycyclic C₆-C₁₂ aryl, branched polycyclic C₆-C₁₂ aryl, fused polycyclic C₆-C₁₂ aryl, branched fused polycyclic C₆-C₁₂ aryl, C₆-C₁₁ aryl, branched C₆-C₁₁ aryl, monocyclic C₆-C₁₁ aryl, polycyclic C₆-C₁₁ aryl, branched polycyclic C₆-C₁₁ aryl, fused polycyclic C₆-C₁₁ aryl, branched fused polycyclic C₆-C₁₁ aryl, C₆-C₉ aryl, branched C₆-C₉ aryl, monocyclic C₆-C₉ aryl, polycyclic C₆-C₉ aryl, branched polycyclic C₆-C₉ aryl, fused polycyclic C₆-C₉ aryl, branched fused polycyclic C₆-C₉ aryl, C₆ aryl, branched C₆ aryl, monocyclic C₆ aryl, C₆-C₅₀ heteroaryl, branched C₆-C₅₀ heteroaryl, monocyclic C₆-C₅₀ heteroaryl, polycyclic C₆-C₅₀ heteroaryl, branched polycyclic C₆-C₅₀ heteroaryl, fused polycyclic C₆-C₅₀ heteroaryl, branched fused polycyclic C₆-C₅₀ heteroaryl, C₆-C₃₀ heteroaryl, branched C₆-C₃₀ heteroaryl, monocyclic C₆-C₃₀ heteroaryl, polycyclic C₆-C₃₀ heteroaryl, branched polycyclic C₆-C₃₀ heteroaryl, fused polycyclic C₆-C₃₀ heteroaryl, branched fused polycyclic C₆-C₃₀ heteroaryl, C₆-C₂₀ heteroaryl, branched C₆-C₂₀ heteroaryl, monocyclic C₆-C₂₀ heteroaryl, polycyclic C₆-C₂₀ heteroaryl, branched polycyclic C₆-C₂₀ heteroaryl, fused polycyclic C₆-C₂₀ heteroaryl, or branched fused polycyclic C₆-C₂₀ heteroaryl, C₆-C₁₂ heteroaryl, branched C₆-C₁₂ heteroaryl, monocyclic C₆-C₁₂ heteroaryl, polycyclic C₆-C₁₂ heteroaryl, branched polycyclic C₆-C₁₂ heteroaryl, fused polycyclic C₆-C₁₂ heteroaryl, branched fused polycyclic C₆-C₁₂ heteroaryl, C₆-C₁₁ heteroaryl, branched C₆-C₁₁ heteroaryl, monocyclic C₆-C₁₁ heteroaryl, polycyclic C₆-C₁₁ heteroaryl, branched polycyclic C₆-C₁₁ heteroaryl, fused polycyclic C₆-C₁₁ heteroaryl, branched fused polycyclic C₆-C₁₁ heteroaryl, C₆-C₉ heteroaryl, branched C₆-C₉ heteroaryl, monocyclic C₆-C₉ heteroaryl, polycyclic C₆-C₉ heteroaryl, branched polycyclic C₆-C₉ heteroaryl, fused polycyclic C₆-C₉ heteroaryl, branched fused polycyclic C₆-C₉ heteroaryl, C₆ heteroaryl, branched C₆ heteroaryl, or monocyclic C₆ heteroaryl.

Exemplary compounds with the structure of Formula I, Formula II, Formula III, Formula III′ or Formula III″ include compounds 1a-1j, 7a-7c, 9a-9n, cKC10′-F, cKC10′-R, cKC9′-F, cKC9′-R, cCK9′-F, cCK9′-R, 20a-20e, 23a-23r, and 23a′-23r′, the structure of which are shown below.

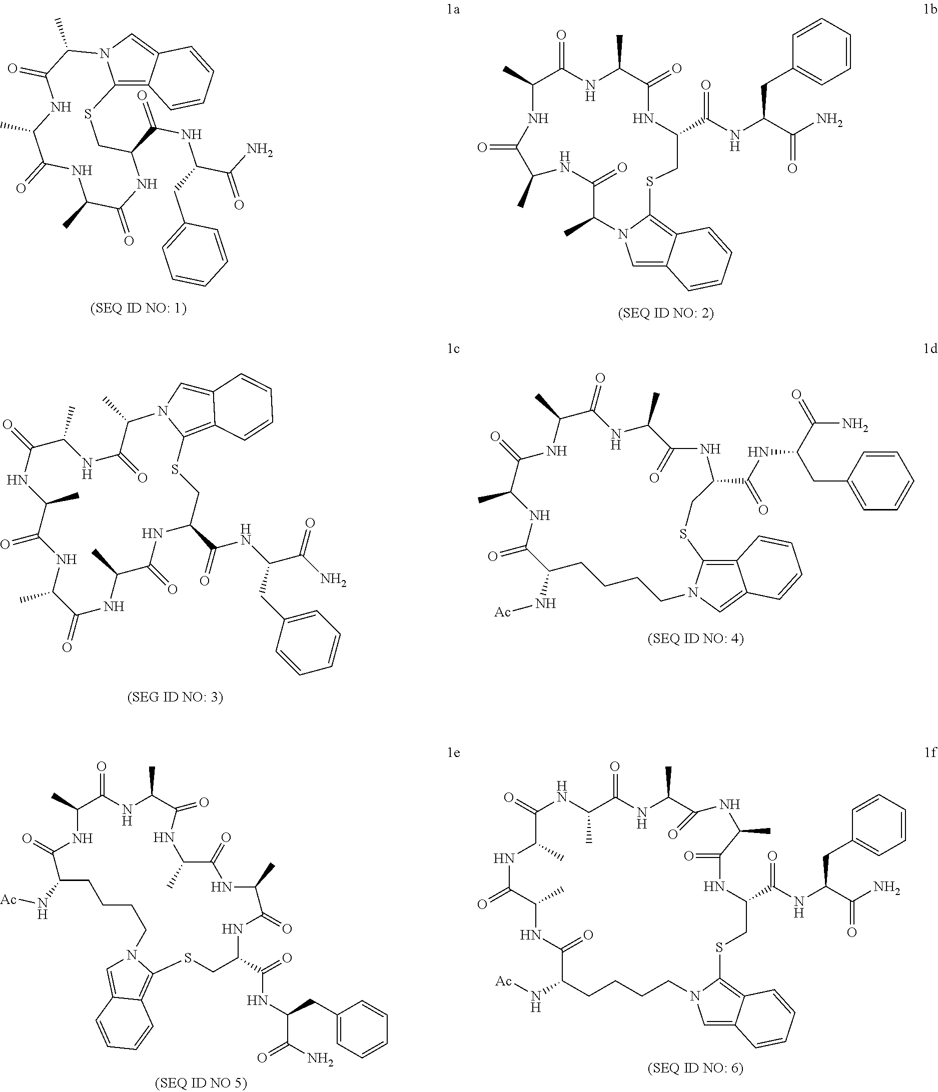

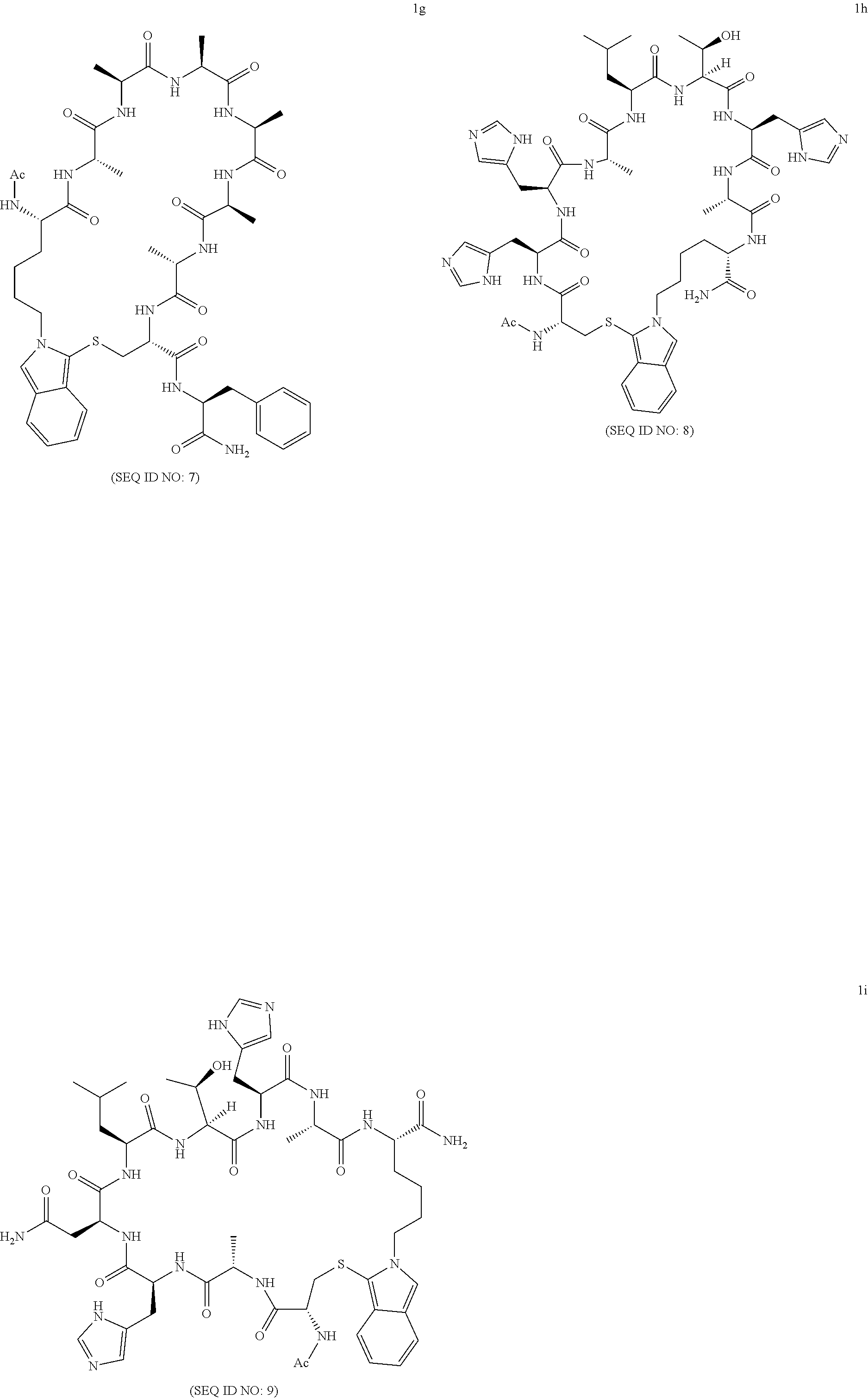

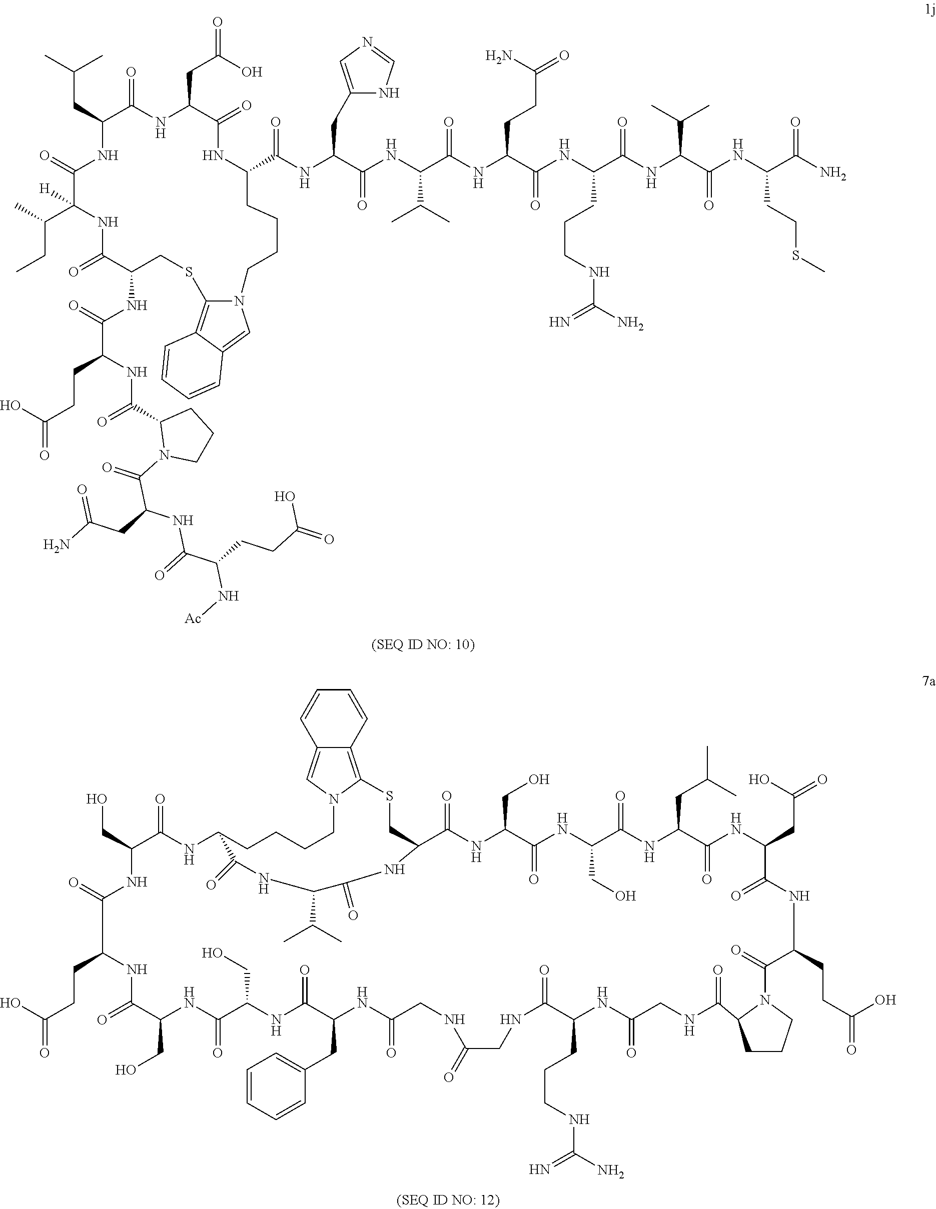

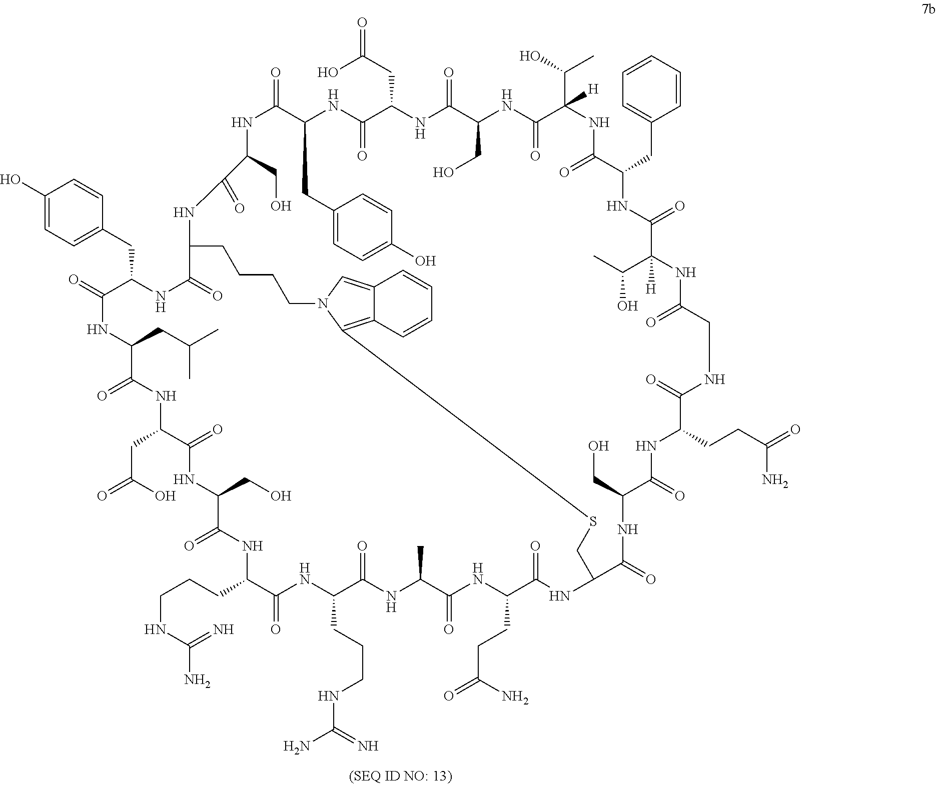 [SMILES: CC(C)C[C@@H]1NC(=O)[C@H](Cc2ccc(O)cc2)NC(=O)C2CCCCn3cc4ccccc4c3SCC(NC(=O)[C@H](CCC(N)=O)NC(=O)[C@H](C)NC(=O)[C@H](CCCNC(=N)N)NC(=O)[C@H](CCCNC(=N)N)NC(=O)[C@H](CO)NC(=O)[C@H](CC(=O)O)NC1=O)C(=O)N[C@@H](CO)C(=O)N[C@@H](CCC(N)=O)C(=O)NCC(=O)N[C@@H]([C@@H](C)O)C(=O)N[C@@H](Cc1ccccc1)C(=O)N[C@@H]([C@@H](C)O)C(=O)N[C@@H](CO)C(=O)N[C@@H](CC(=O)O)C(=O)N[C@@H](Cc1ccc(O)cc1)C(=O)N[C@@H](CO)C(=O)N2]

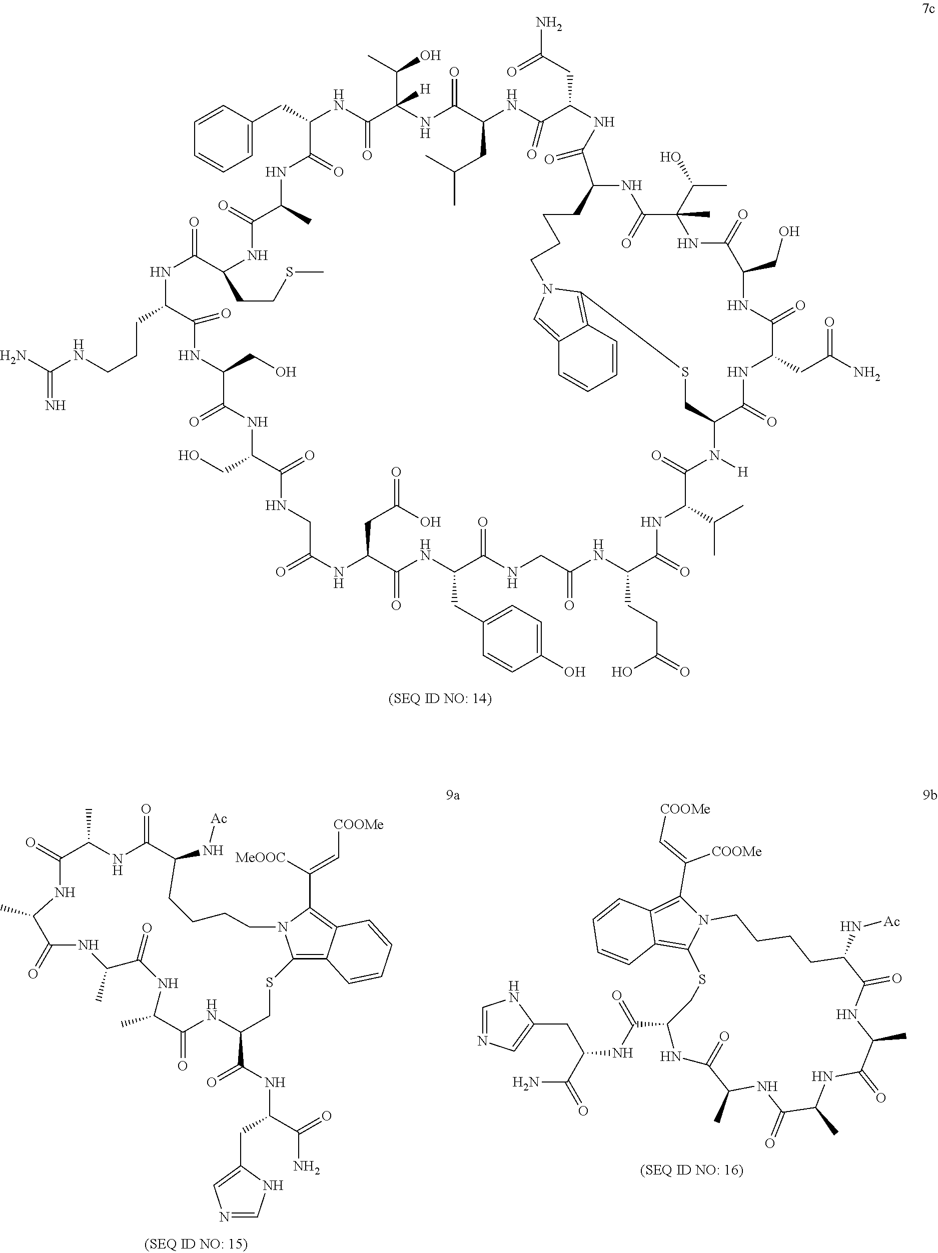

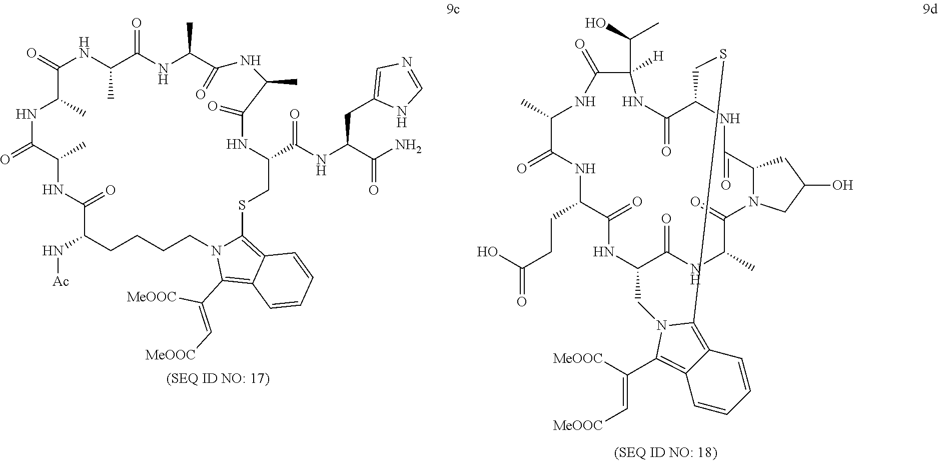

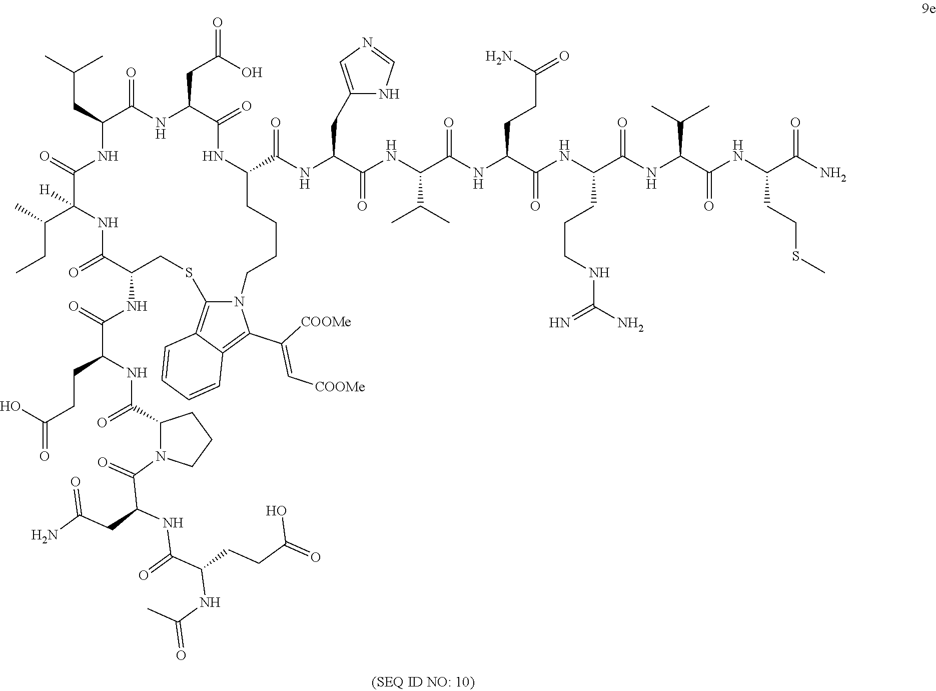 [SMILES: CC[C@H](C)[C@@H]1NC(=O)[C@@H](NC(=O)[C@H](CCC(=O)O)NC(=O)[C@@H]2CCCN2C(=O)[C@H](CC(N)=O)NC(=O)[C@H](CCC(=O)O)NC(C)=O)CSc2c3ccccc3c(/C(=C/C(=O)OC)C(=O)OC)n2CCCC[C@@H](C(=O)N[C@@H](Cc2cnc[nH]2)C(=O)N[C@H](C(=O)N[C@@H](CCC(N)=O)C(=O)N[C@@H](CCCNC(=N)N)C(=O)N[C@H](C(=O)N[C@@H](CCSC)C(N)=O)C(C)C)C(C)C)NC(=O)[C@H](CC(=O)O)NC(=O)[C@H](CC(C)C)NC1=O]

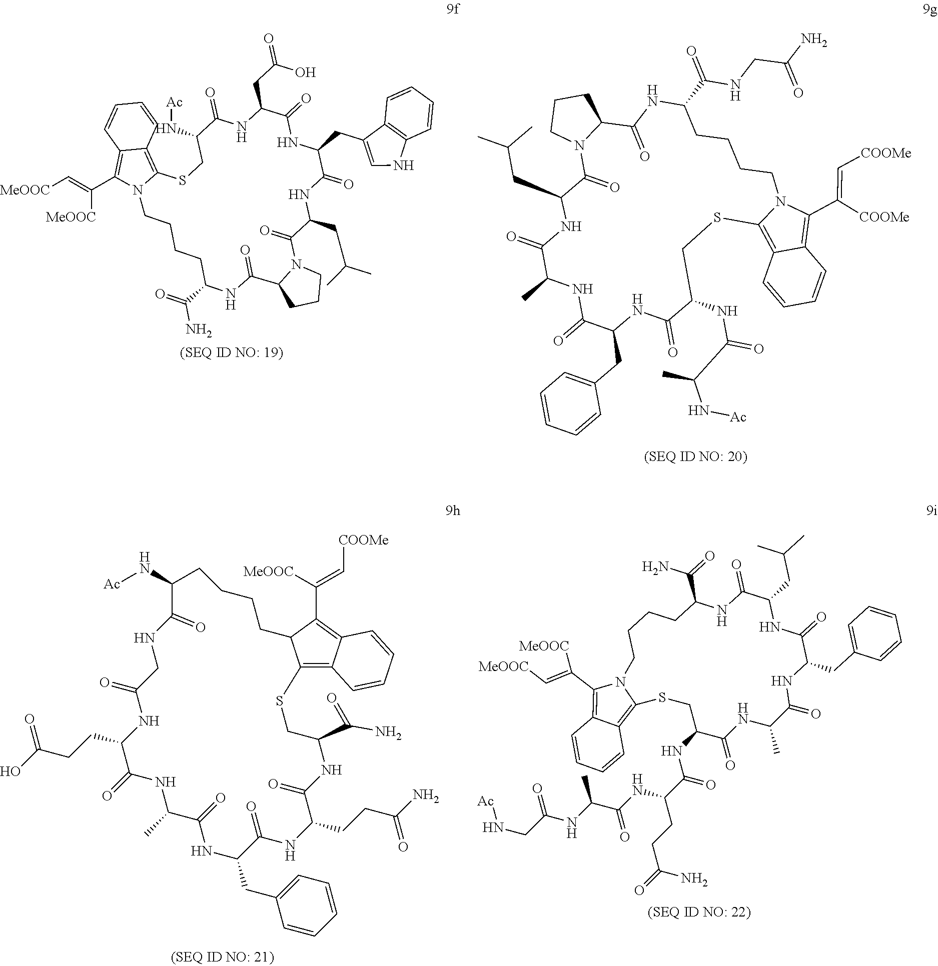

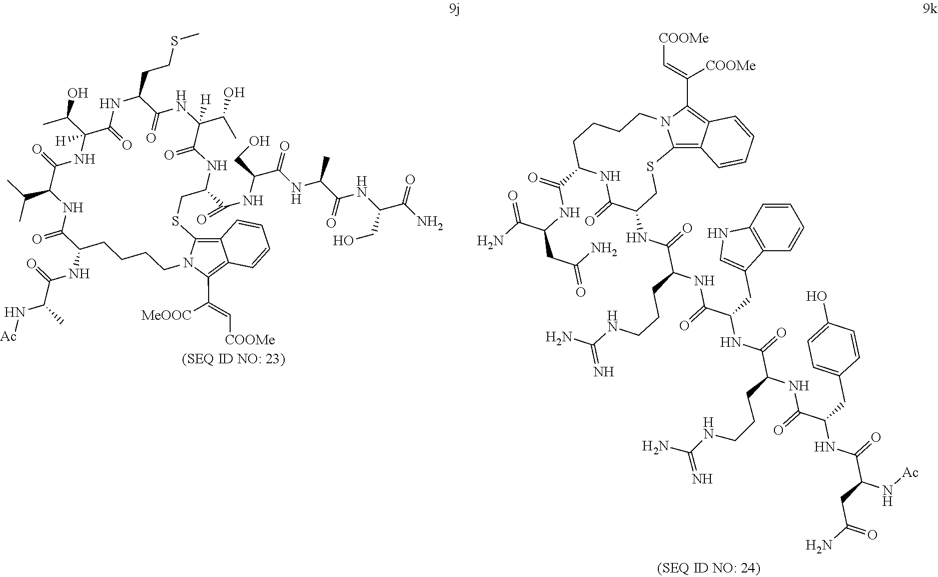 [SMILES: COC(=O)/C=C(\C(=O)OC)c1c2ccccc2c2n1CCCC[C@H](NC(=O)[C@H](C)NC(C)=O)C(=O)N[C@@H](C(C)C)C(=O)N[C@@H]([C@@H](C)O)C(=O)N[C@@H](CCSC)C(=O)N[C@@H]([C@@H](C)O)C(=O)N[C@H](C(=O)N[C@@H](CO)C(=O)N[C@@H](C)C(=O)N[C@@H](CO)C(N)=O)CS2 ; COC(=O)/C=C(\C(=O)OC)c1c2ccccc2c2n1CCCC[C@@H](C(=O)N[C@@H](CC(N)=O)C(N)=O)NC(=O)[C@@H](NC(=O)[C@H](CCCNC(=N)N)NC(=O)[C@H](Cc1c[nH]c3ccccc13)NC(=O)[C@H](CCCNC(=N)N)NC(=O)[C@H](Cc1ccc(O)cc1)NC(=O)[C@H](CC(N)=O)NC(C)=O)CS2]

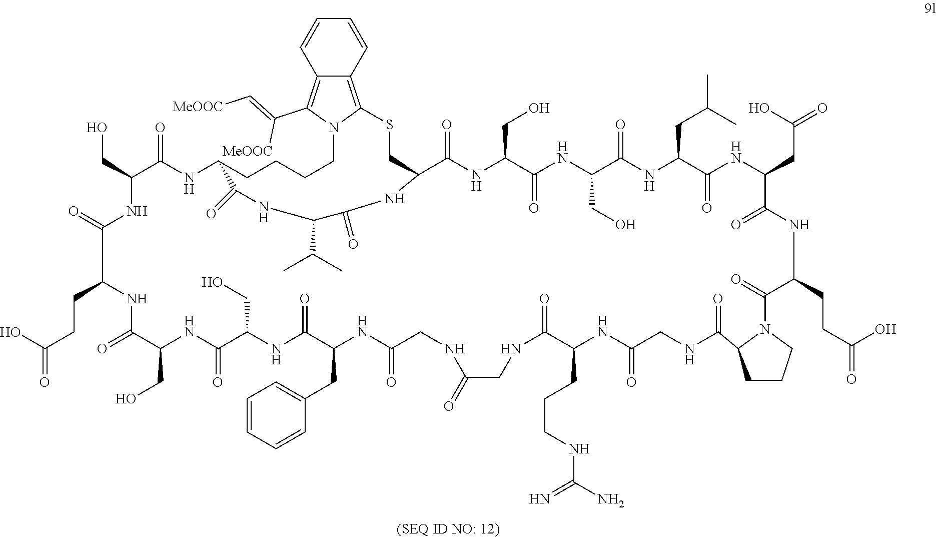 [SMILES: COC(=O)/C=C(\C(=O)OC)c1c2ccccc2c2n1CCCC[C@@H]1NC(=O)[C@H](CO)NC(=O)[C@H](CCC(=O)O)NC(=O)[C@H](CO)NC(=O)[C@H](CO)NC(=O)[C@H](Cc3ccccc3)NC(=O)CNC(=O)CNC(=O)[C@H](CCCNC(=N)N)NC(=O)CNC(=O)[C@@H]3CCCN3C(=O)[C@H](CCC(=O)O)NC(=O)[C@H](CC(=O)O)NC(=O)[C@H](CC(C)C)NC(=O)[C@H](CO)NC(=O)[C@H](CO)NC(=O)[C@H](CS2)NC(=O)[C@H](C(C)C)NC1=O]

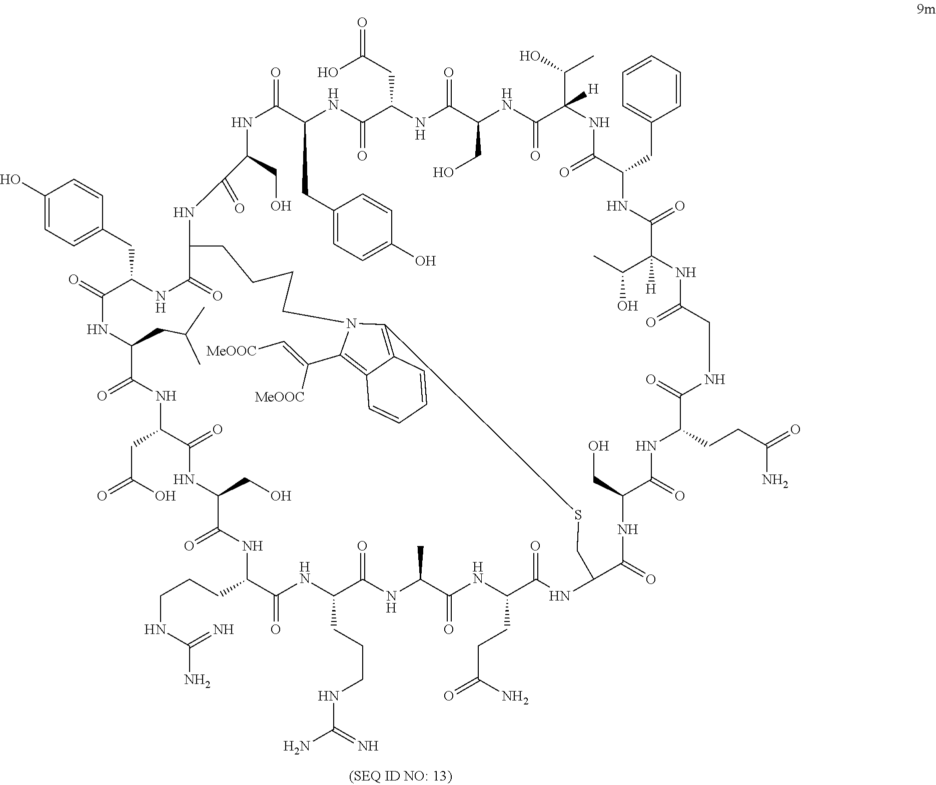 [SMILES: COC(=O)/C=C(\C(=O)OC)c1c2ccccc2c2n1CCCCC1NC(=O)[C@H](CO)NC(=O)[C@H](Cc3ccc(O)cc3)NC(=O)[C@H](CC(=O)O)NC(=O)[C@H](CO)NC(=O)[C@H]([C@@H](C)O)NC(=O)[C@H](Cc3ccccc3)NC(=O)[C@H]([C@@H](C)O)NC(=O)CNC(=O)[C@H](CCC(N)=O)NC(=O)[C@H](CO)NC(=O)C(CS2)NC(=O)[C@H](CCC(N)=O)NC(=O)[C@H](C)NC(=O)[C@H](CCCNC(=N)N)NC(=O)[C@H](CCCNC(=N)N)NC(=O)[C@H](CO)NC(=O)[C@H](CC(=O)O)NC(=O)[C@H](CC(C)C)NC(=O)[C@H](Cc2ccc(O)cc2)NC1=O]

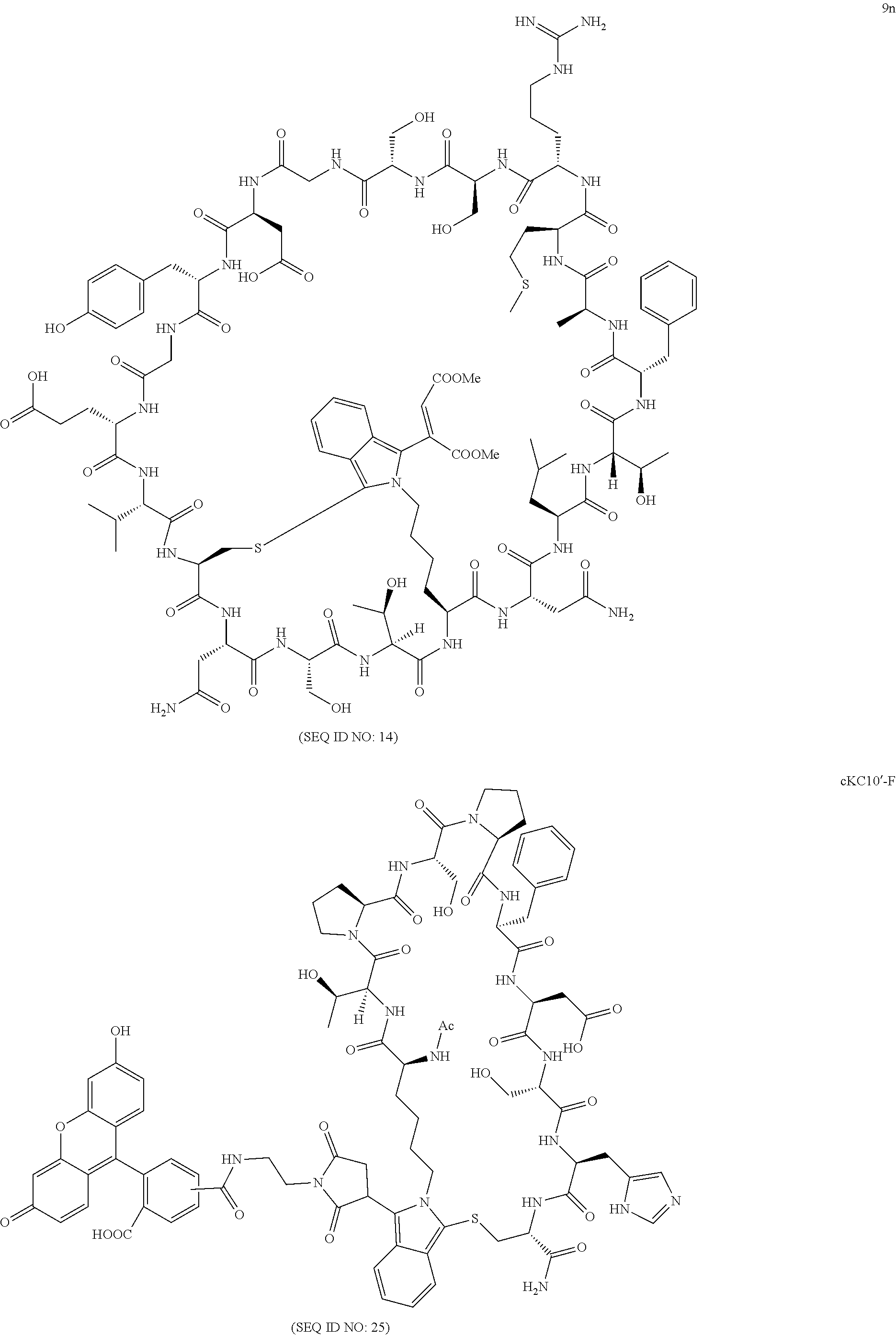

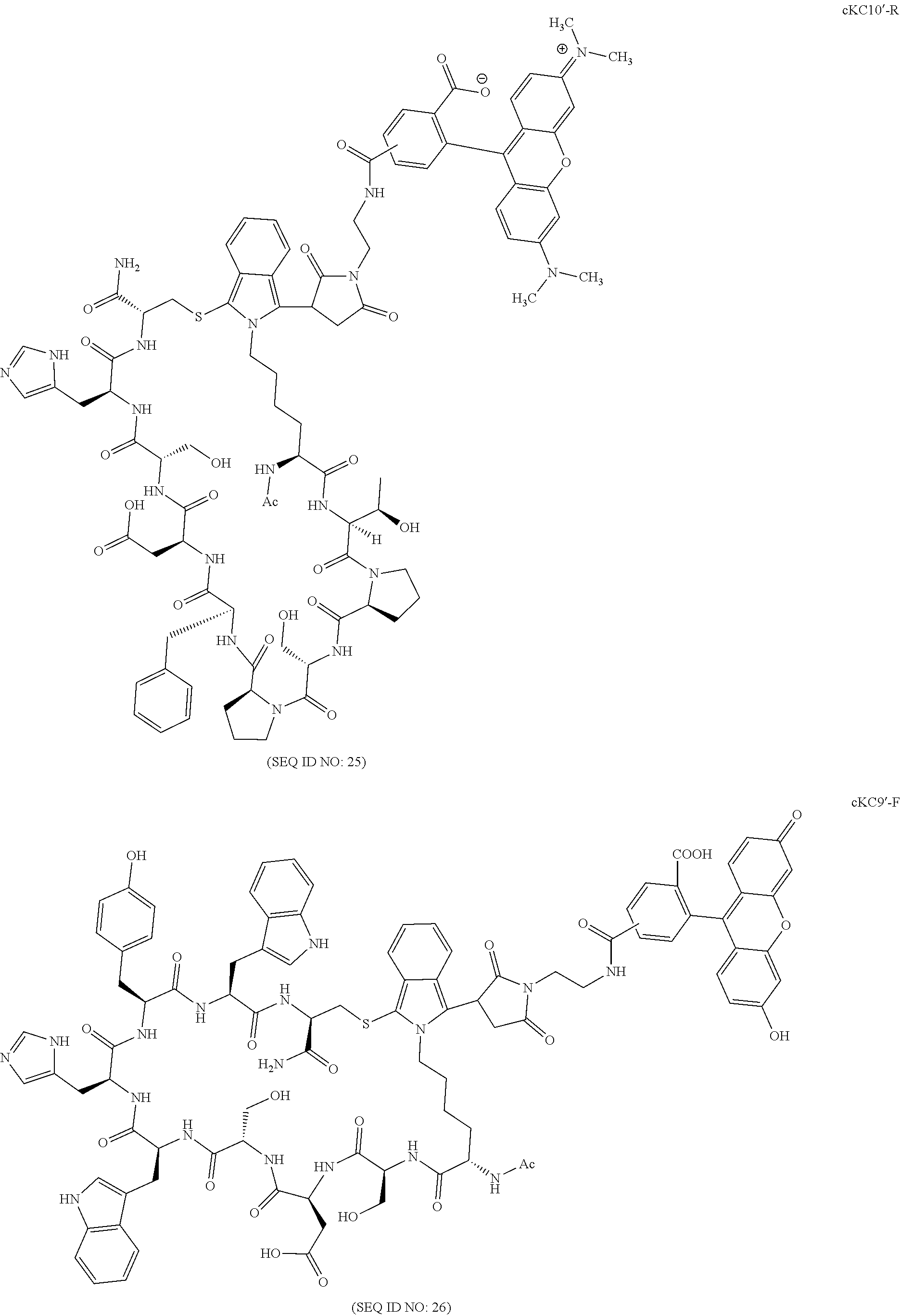

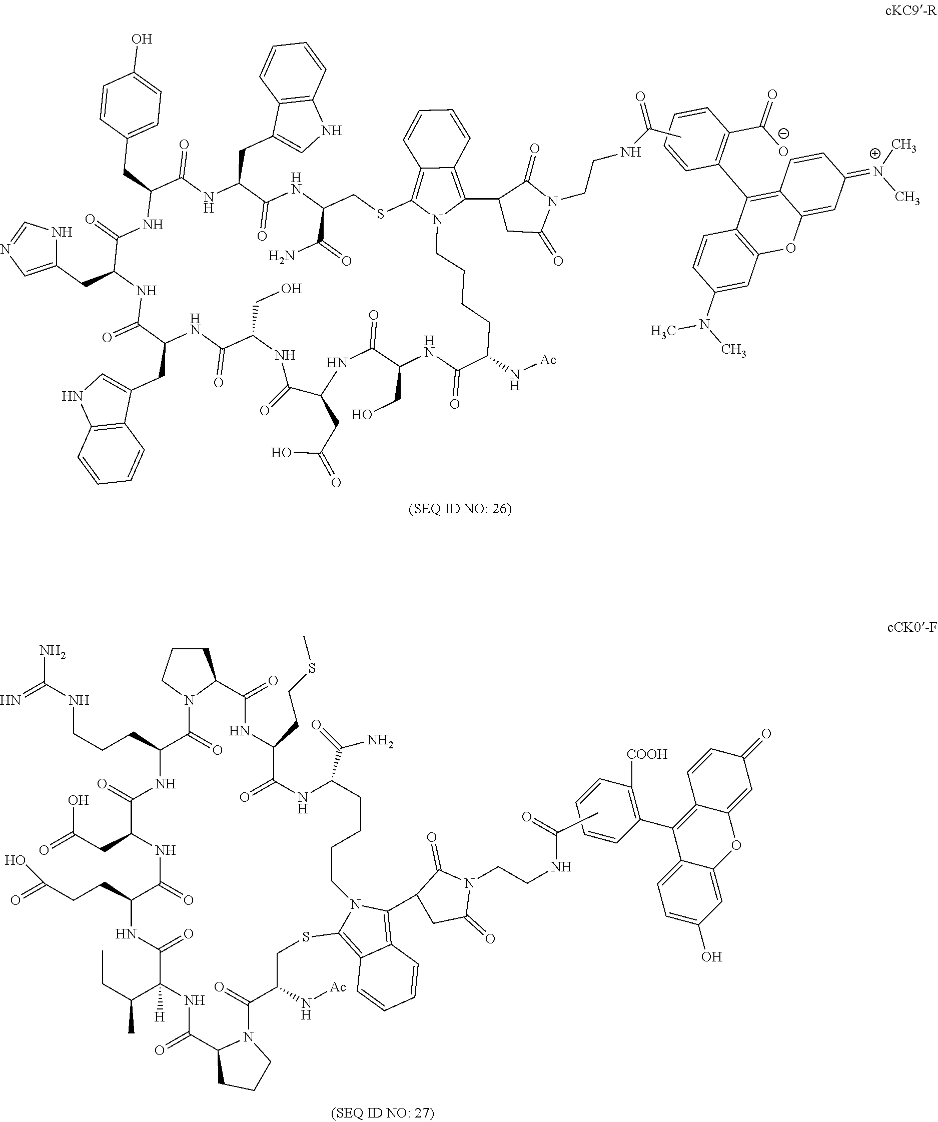

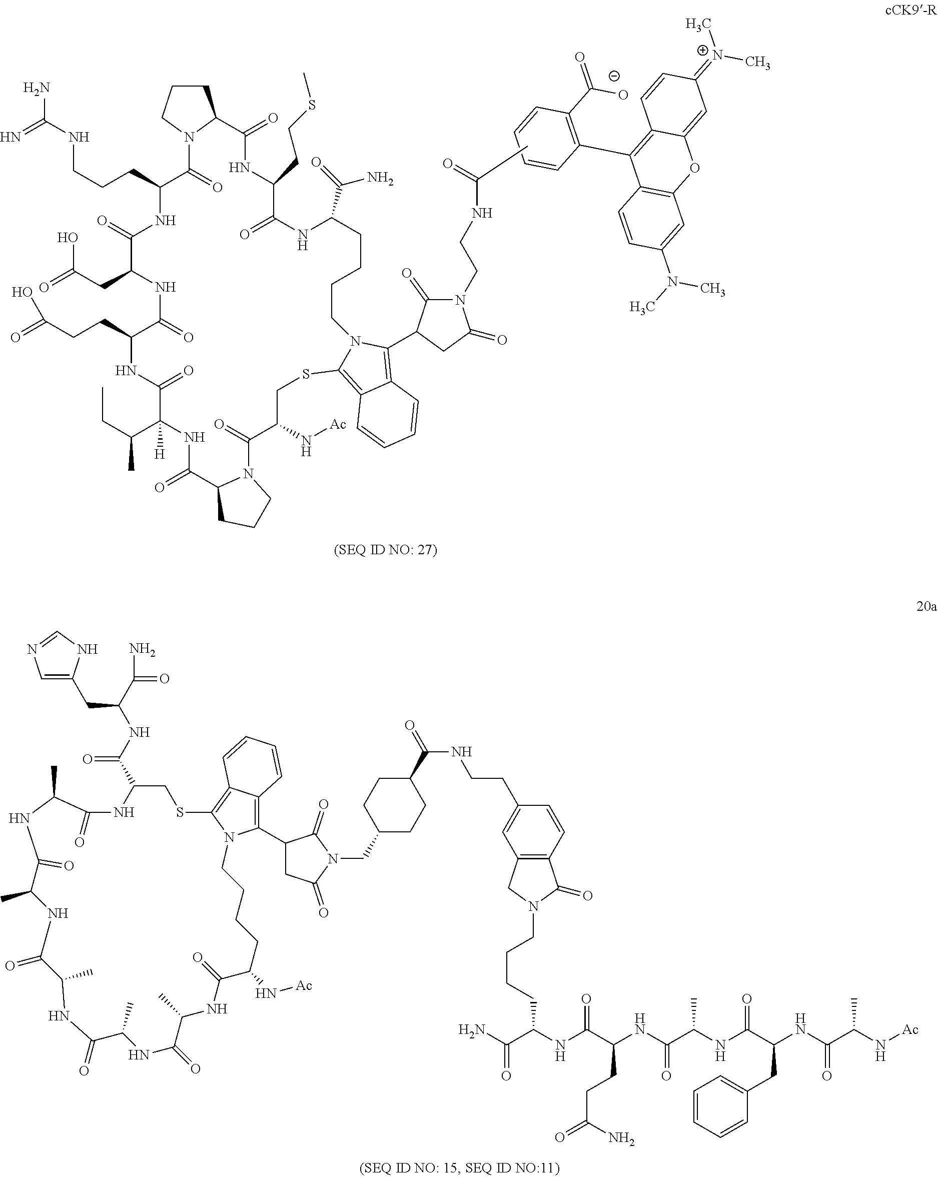

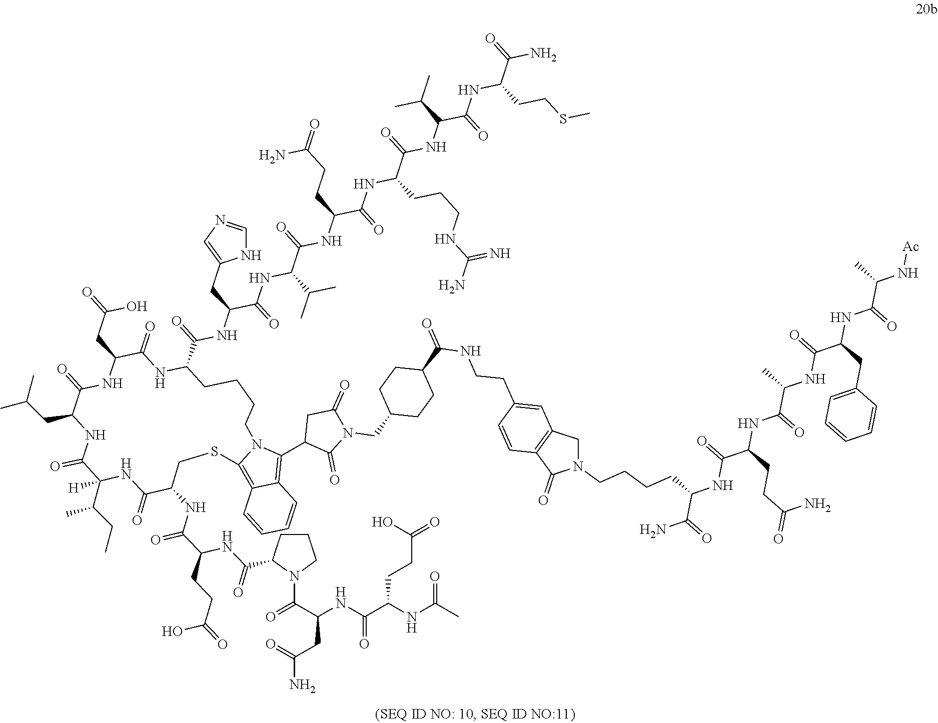

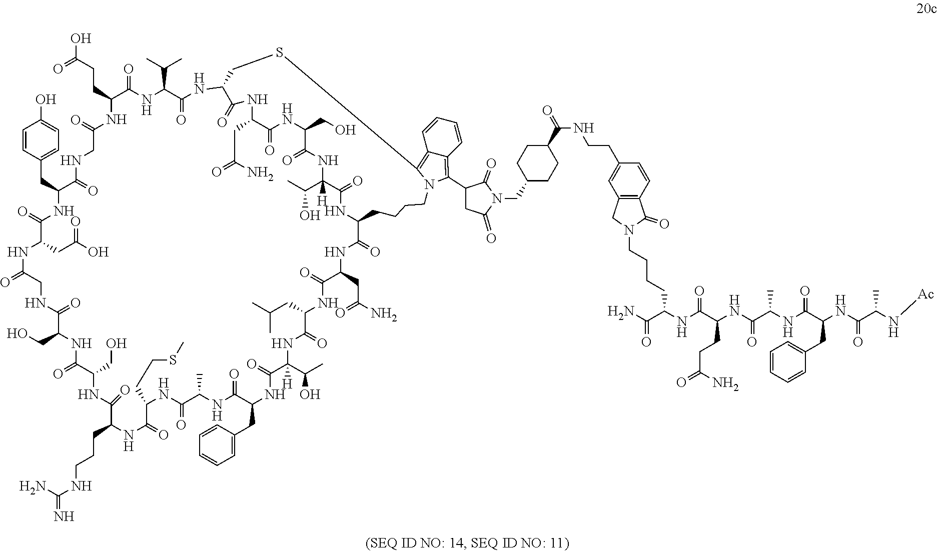

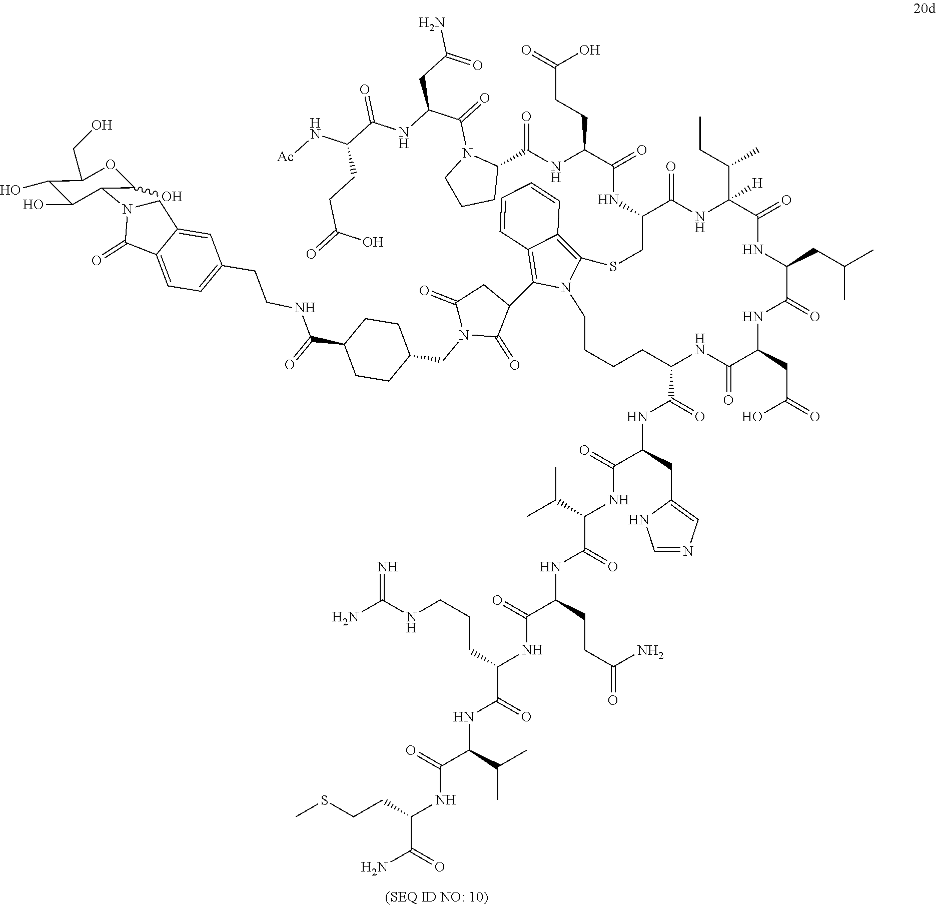 [SMILES: CC[C@H](C)[C@@H]1NC(=O)[C@@H](NC(=O)[C@H](CCC(=O)O)NC(=O)[C@@H]2CCCN2C(=O)[C@H](CC(N)=O)NC(=O)[C@H](CCC(=O)O)NC(C)=O)CSc2c3ccccc3c(C3CC(=O)N(C[C@H]4CC[C@H](C(=O)NCCc5ccc6c(c5)CN(C5C(O)OC(CO)C(O)C5O)C6=O)CC4)C3=O)n2CCCC[C@@H](C(=O)N[C@@H](Cc2cnc[nH]2)C(=O)N[C@H](C(=O)N[C@@H](CCC(N)=O)C(=O)N[C@@H](CCCNC(=N)N)C(=O)N[C@H](C(=O)N[C@@H](CCSC)C(N)=O)C(C)C)C(C)C)NC(=O)[C@H](CC(=O)O)NC(=O)[C@H](CC(C)C)NC1=O]

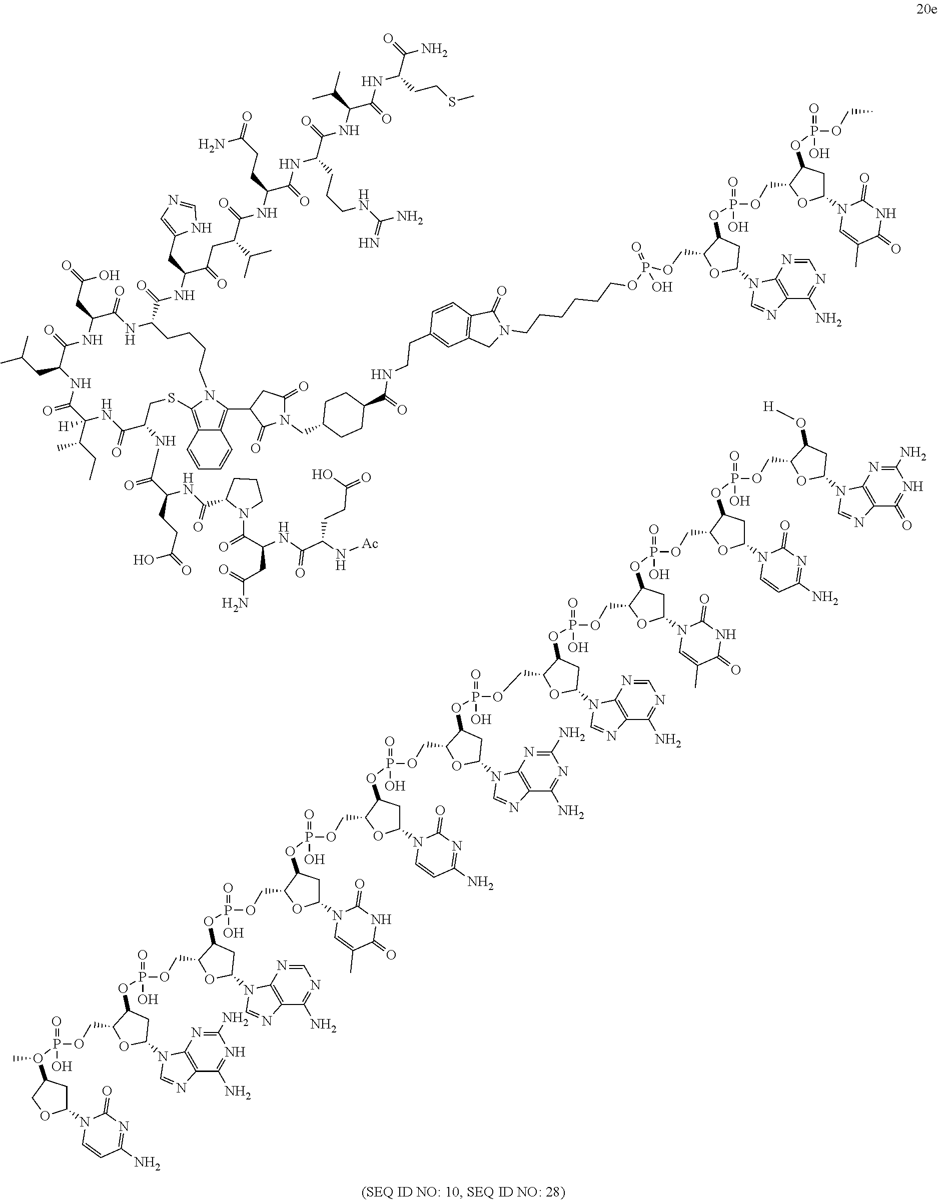

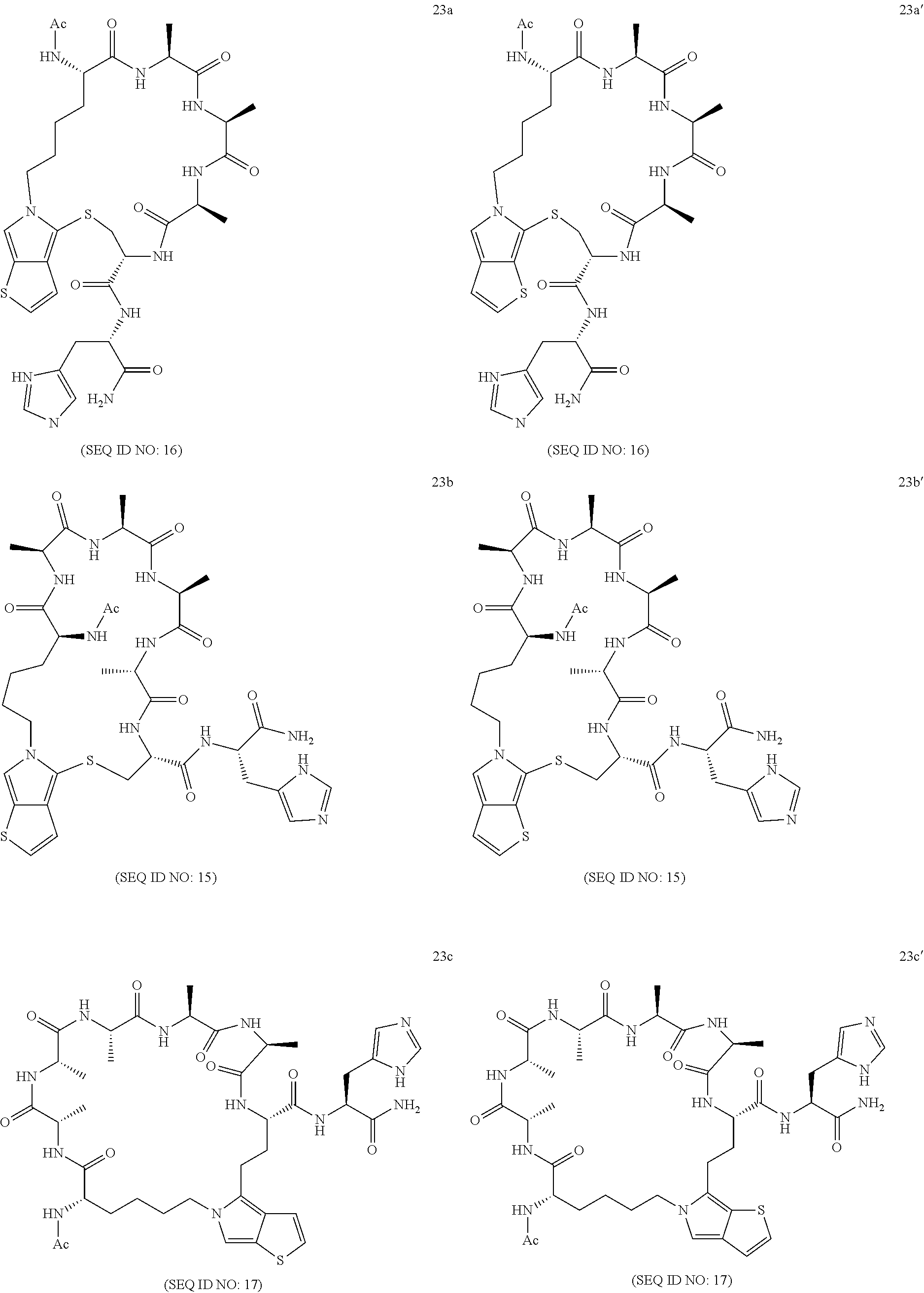

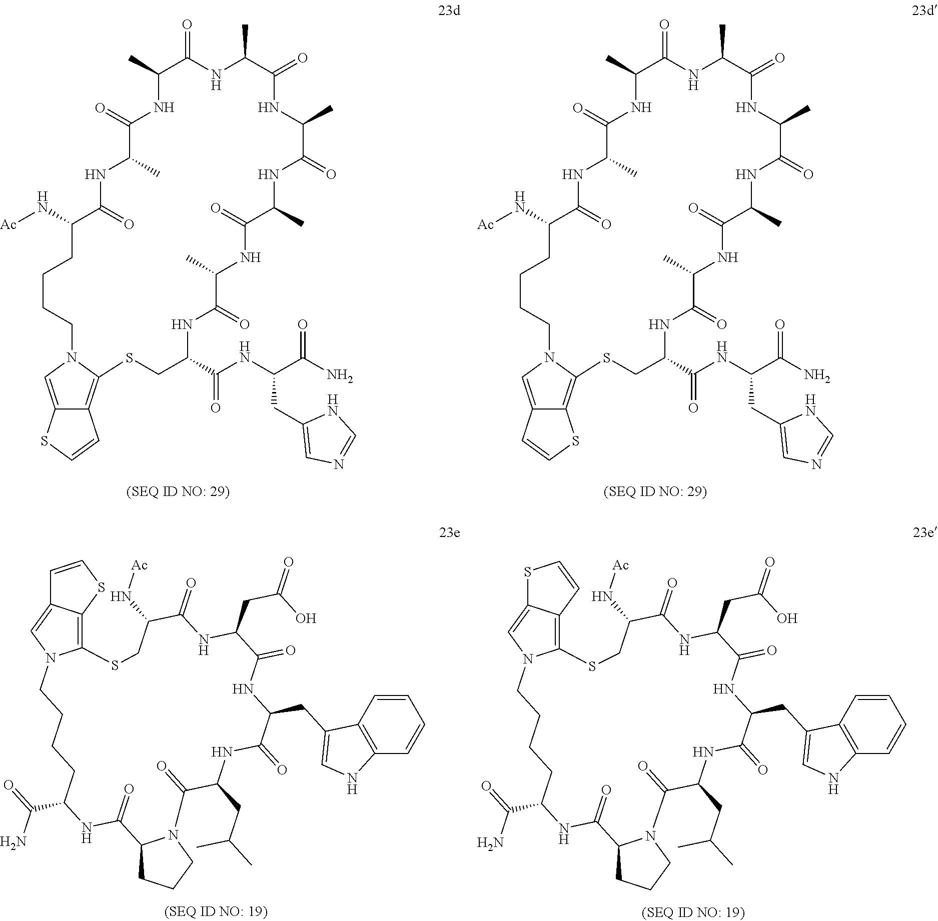

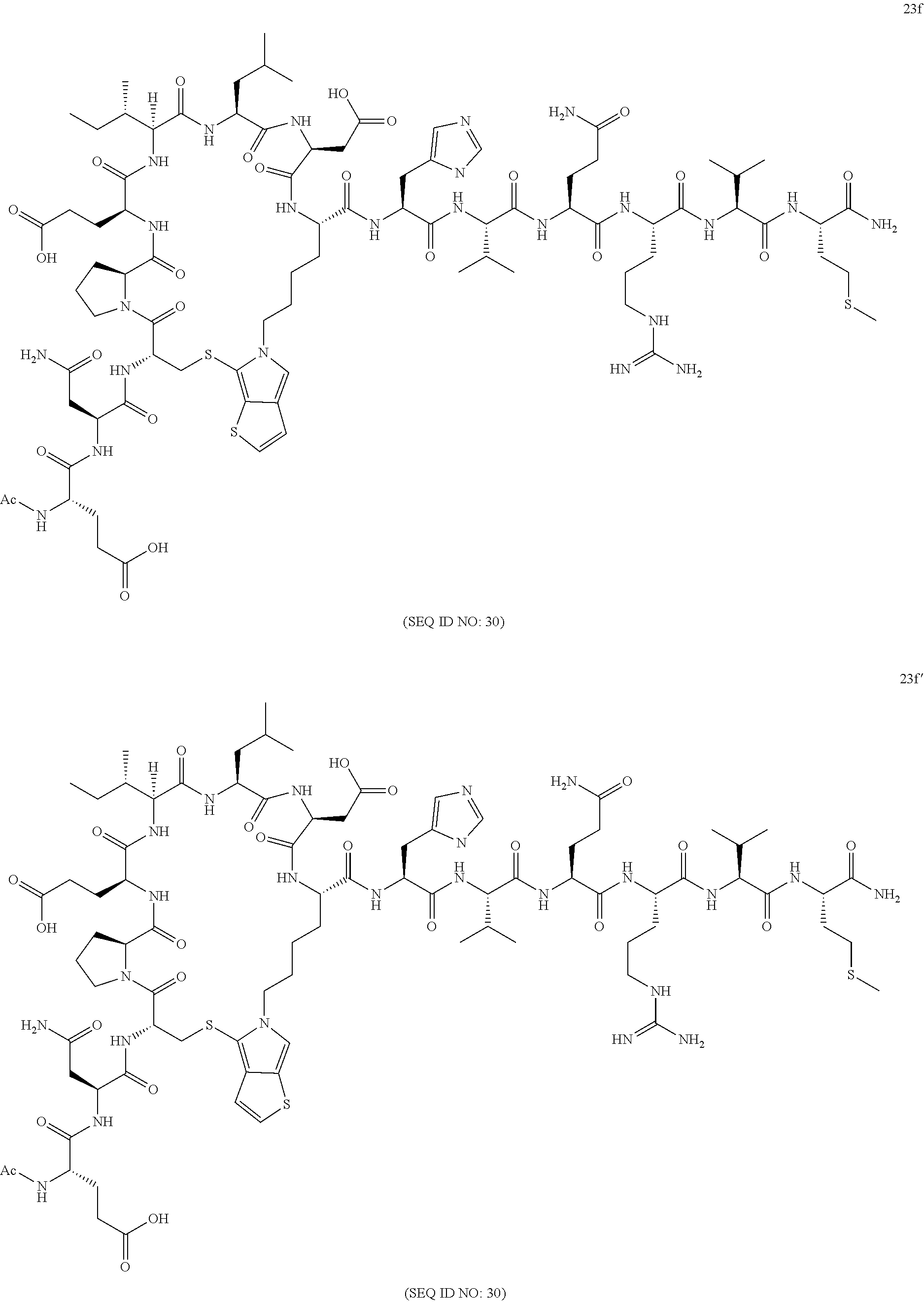

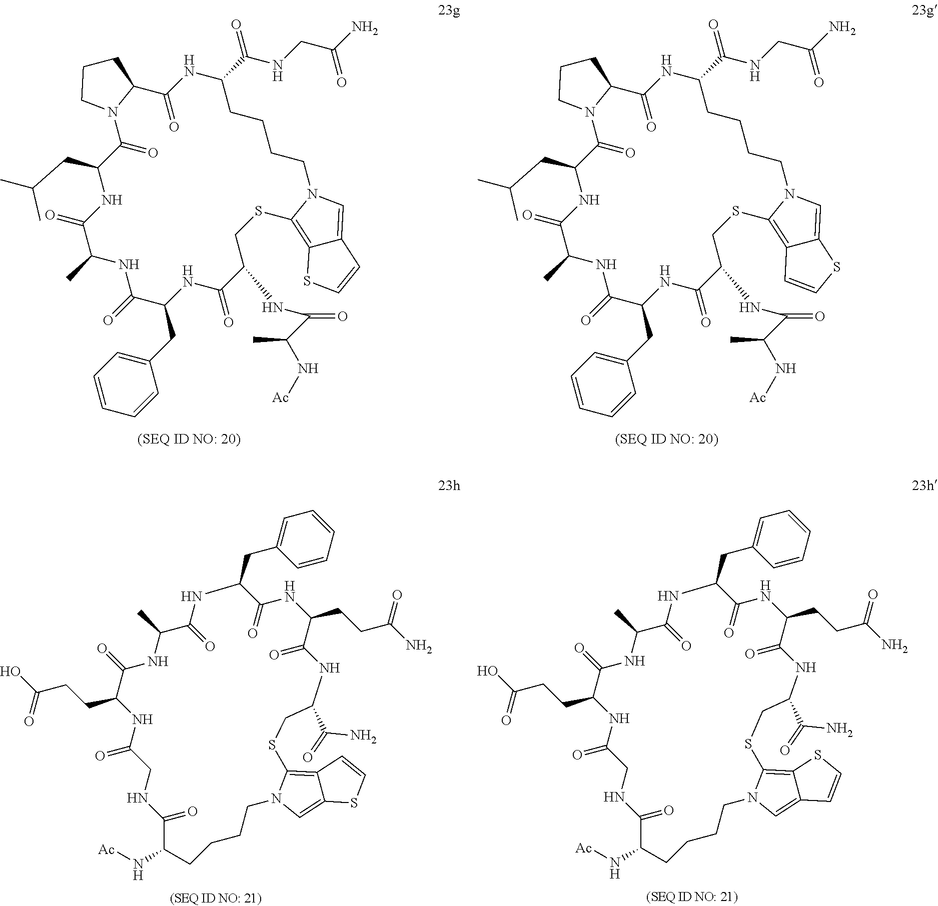

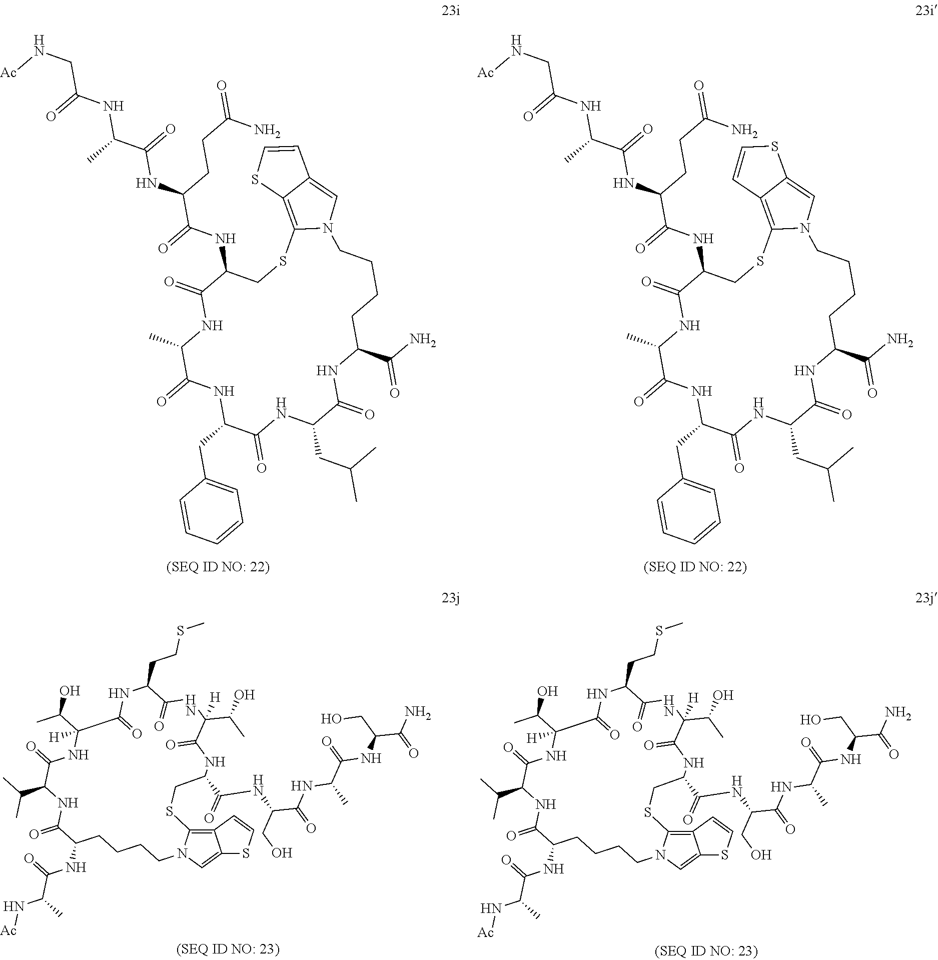

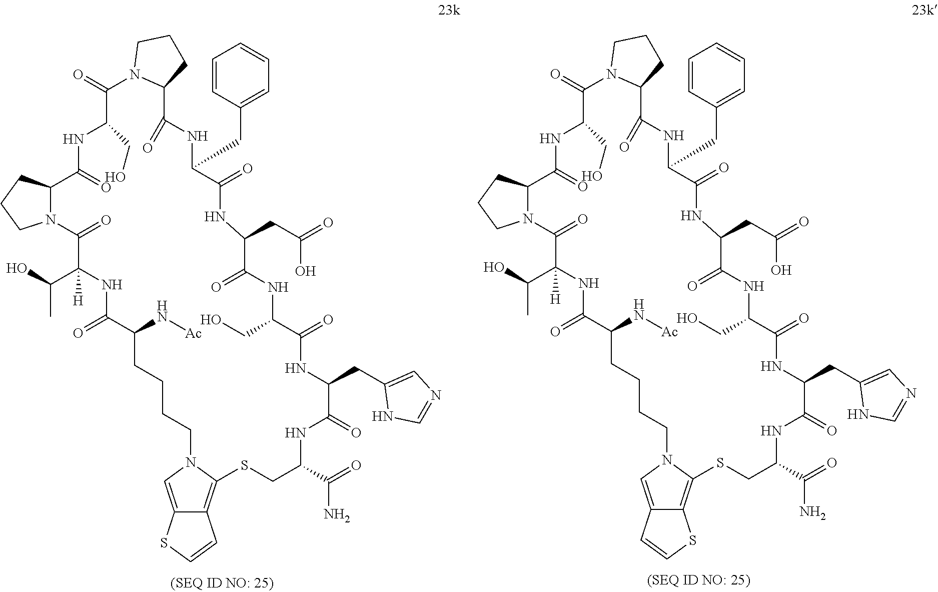 [SMILES: CC(=O)N[C@H]1CCCCn2cc3sccc3c2SC[C@@H](C(N)=O)NC(=O)[C@H](Cc2cnc[nH]2)NC(=O)[C@H](CO)NC(=O)[C@H](CC(=O)O)NC(=O)[C@H](Cc2ccccc2)NC(=O)[C@@H]2CCCN2C(=O)[C@H](CO)NC(=O)[C@@H]2CCCN2C(=O)[C@H]([C@@H](C)O)NC1=O ; CC(=O)N[C@H]1CCCCn2cc3ccsc3c2SC[C@@H](C(N)=O)NC(=O)[C@H](Cc2cnc[nH]2)NC(=O)[C@H](CO)NC(=O)[C@H](CC(=O)O)NC(=O)[C@H](Cc2ccccc2)NC(=O)[C@@H]2CCCN2C(=O)[C@H](CO)NC(=O)[C@@H]2CCCN2C(=O)[C@H]([C@@H](C)O)NC1=O]

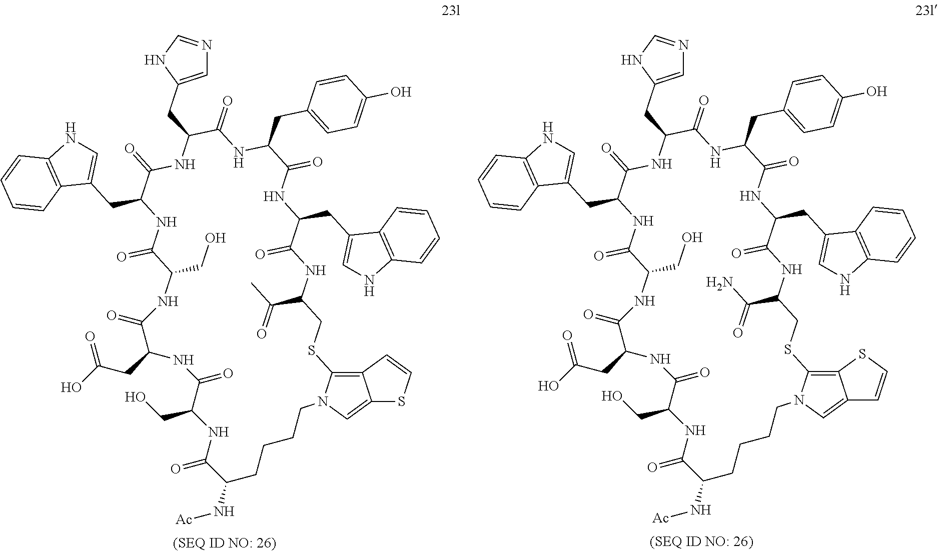

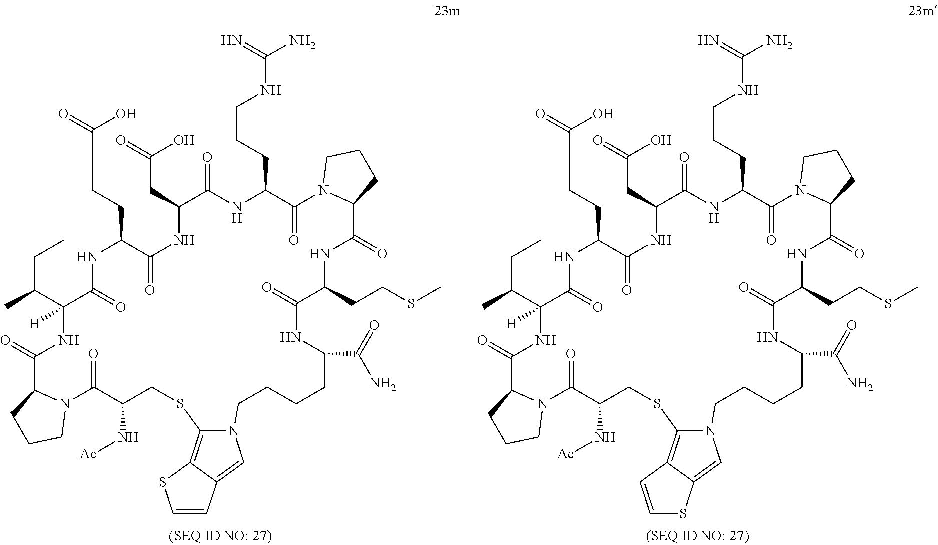 [SMILES: CC[C@H](C)[C@@H]1NC(=O)[C@@H]2CCCN2C(=O)[C@@H](NC(C)=O)CSc2c3sccc3cn2CCCC[C@@H](C(N)=O)NC(=O)[C@H](CCSC)NC(=O)[C@@H]2CCCN2C(=O)[C@H](CCCNC(=N)N)NC(=O)[C@H](CC(=O)O)NC(=O)[C@H](CCC(=O)O)NC1=O ; CC[C@H](C)[C@@H]1NC(=O)[C@@H]2CCCN2C(=O)[C@@H](NC(C)=O)CSc2c3ccsc3cn2CCCC[C@@H](C(N)=O)NC(=O)[C@H](CCSC)NC(=O)[C@@H]2CCCN2C(=O)[C@H](CCCNC(=N)N)NC(=O)[C@H](CC(=O)O)NC(=O)[C@H](CCC(=O)O)NC1=O]

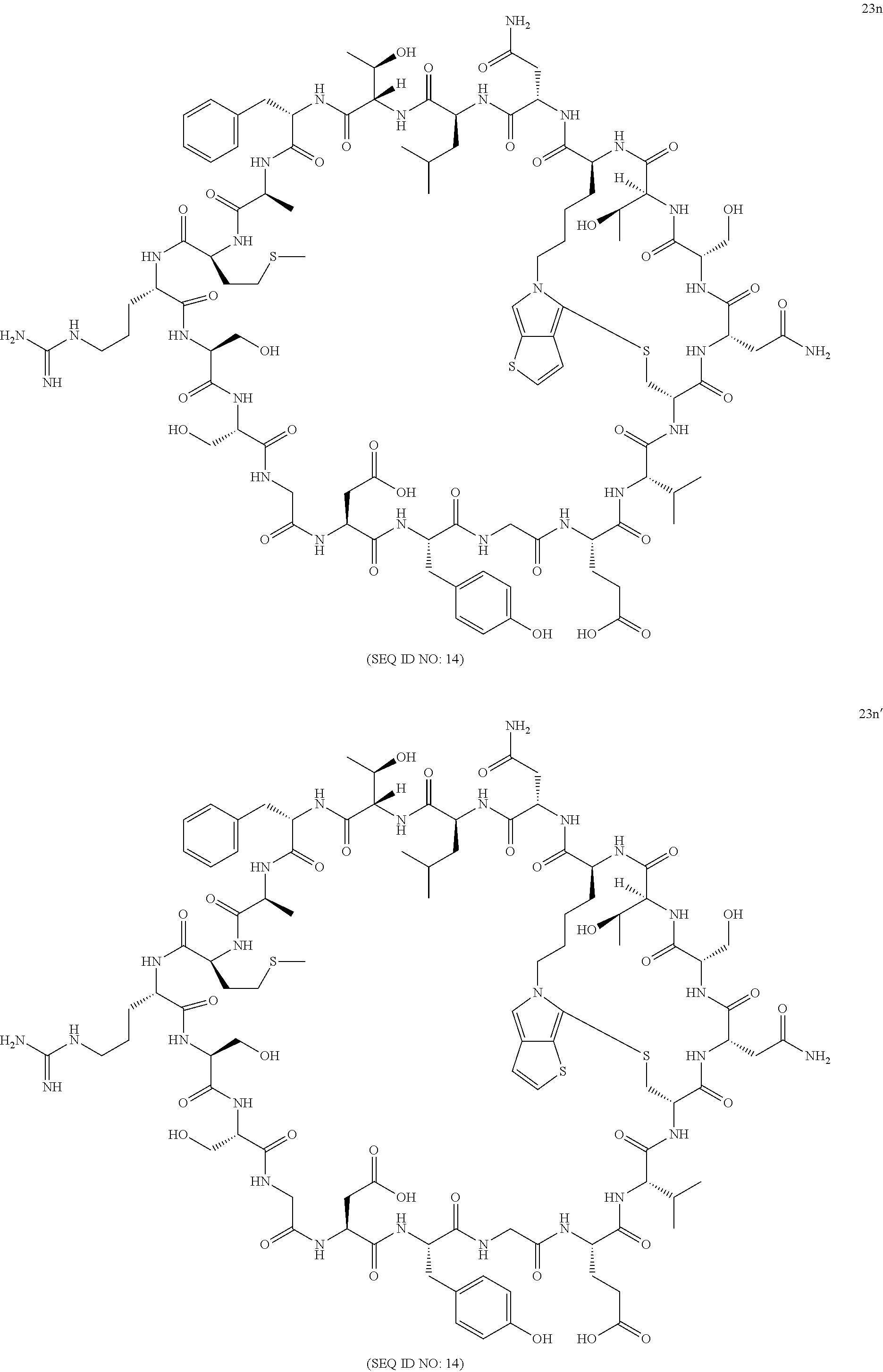

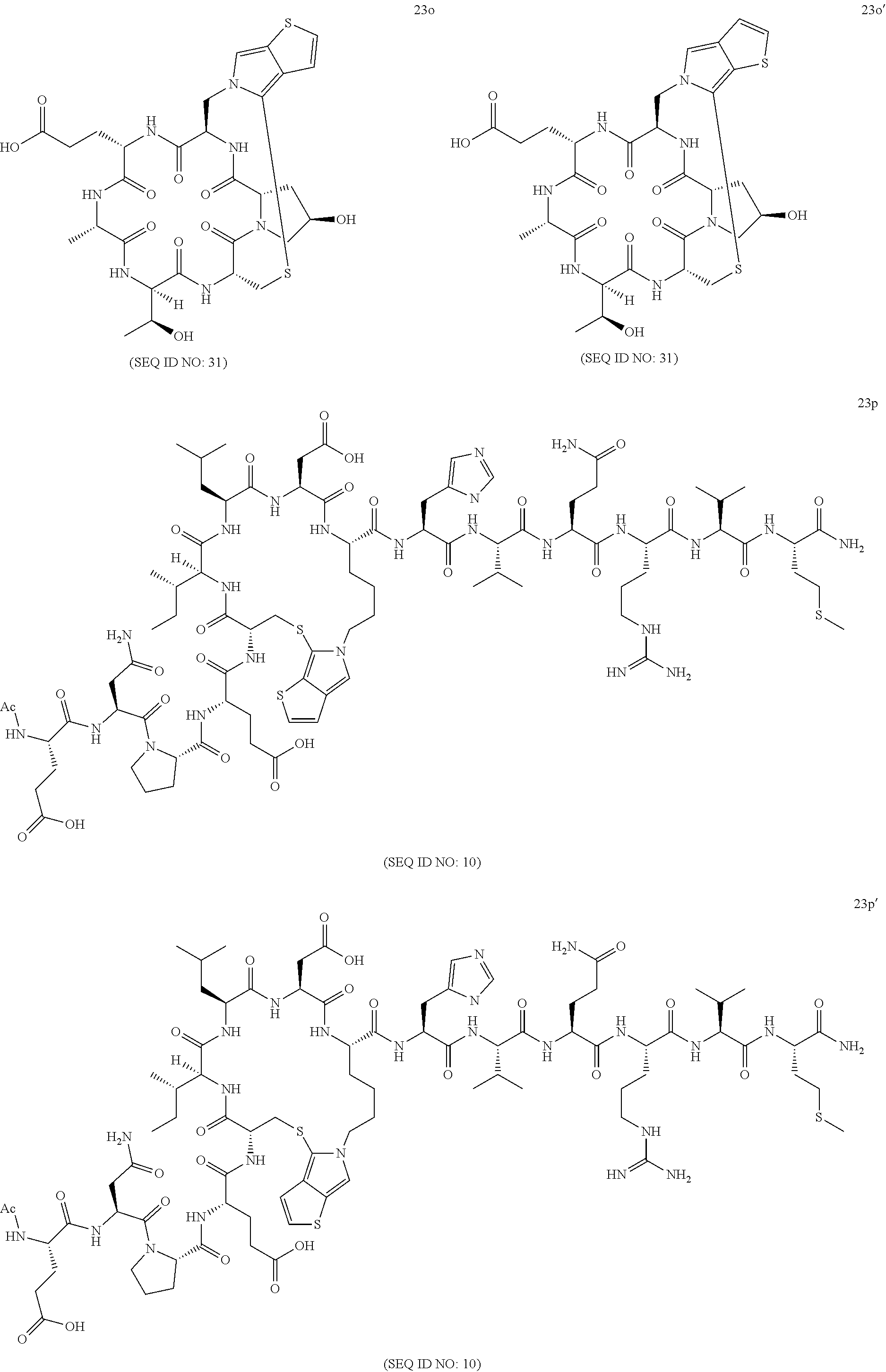

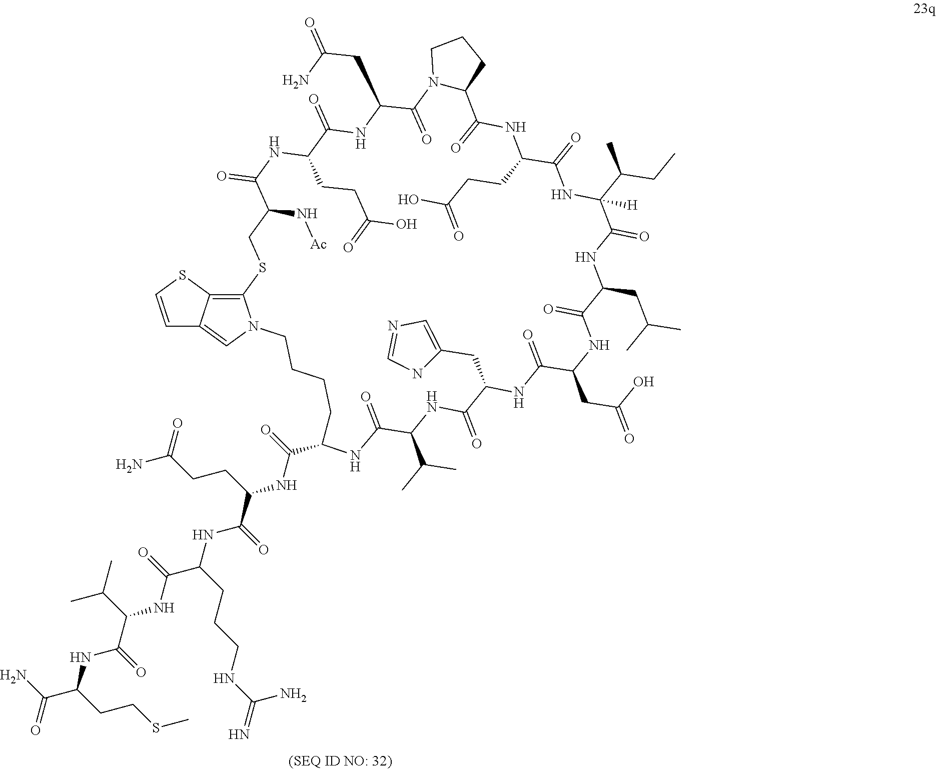

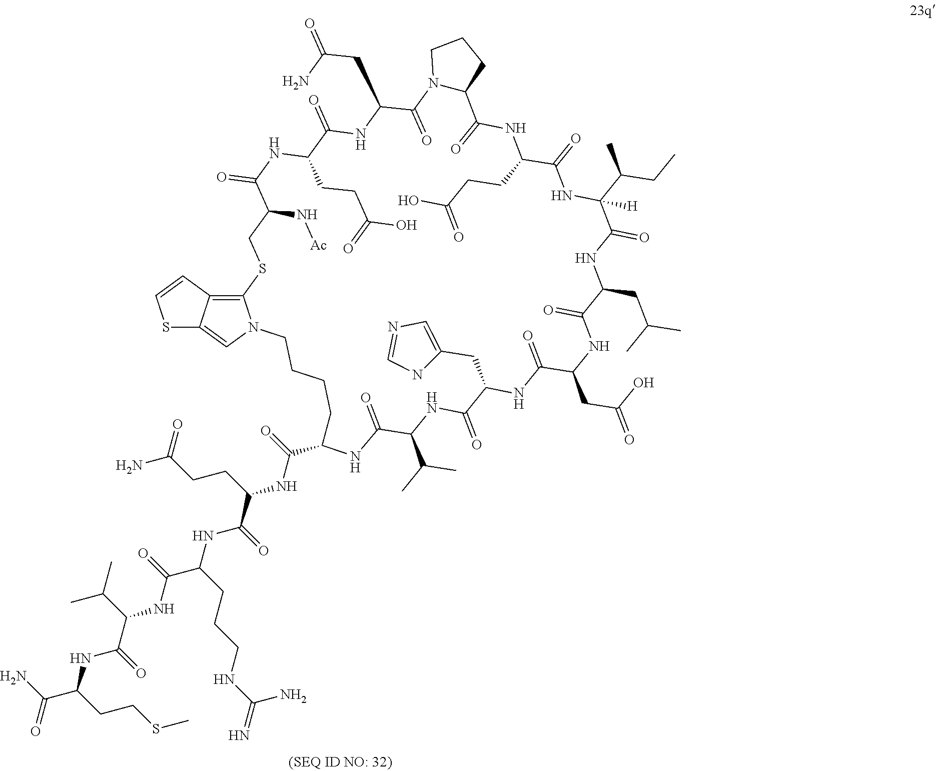

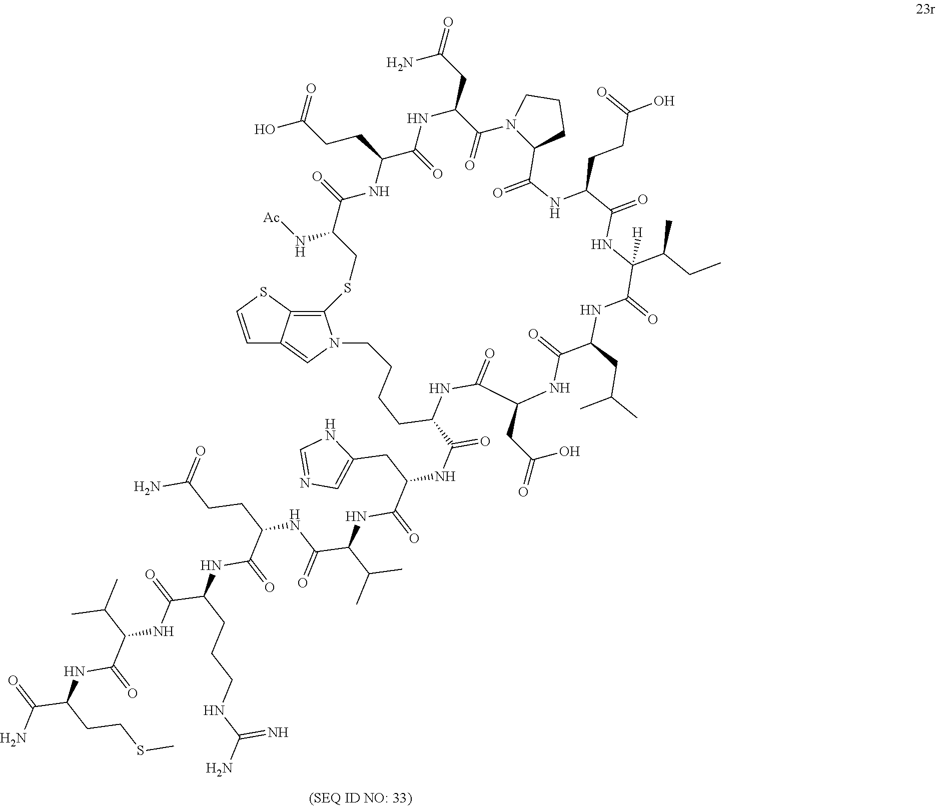 [SMILES: CC[C@H](C)[C@@H]1NC(=O)[C@H](CCC(=O)O)NC(=O)[C@@H]2CCCN2C(=O)[C@H](CC(N)=O)NC(=O)[C@H](CCC(=O)O)NC(=O)[C@@H](NC(C)=O)CSc2c3sccc3cn2CCCC[C@@H](C(=O)N[C@@H](Cc2cnc[nH]2)C(=O)N[C@H](C(=O)N[C@@H](CCC(N)=O)C(=O)N[C@@H](CCCNC(=N)N)C(=O)N[C@H](C(=O)N[C@@H](CCSC)C(N)=O)C(C)C)C(C)C)NC(=O)[C@H](CC(=O)O)NC(=O)[C@H](CC(C)C)NC1=O]

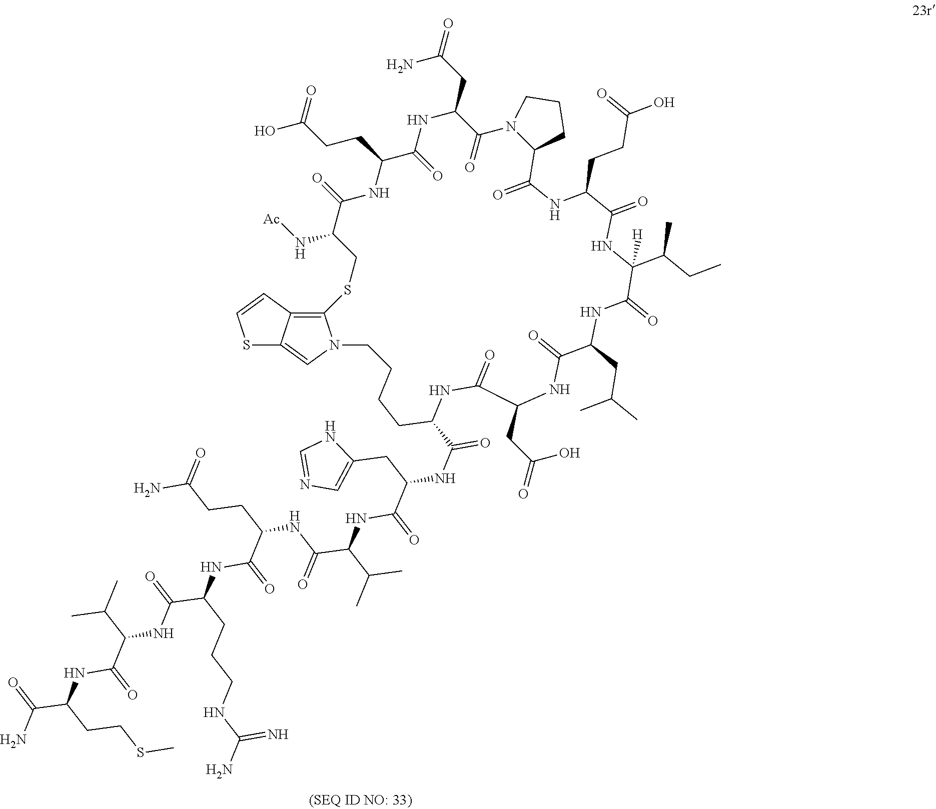 [SMILES: CC[C@H](C)[C@@H]1NC(=O)[C@H](CCC(=O)O)NC(=O)[C@@H]2CCCN2C(=O)[C@H](CC(N)=O)NC(=O)[C@H](CCC(=O)O)NC(=O)[C@@H](NC(C)=O)CSc2c3ccsc3cn2CCCC[C@@H](C(=O)N[C@@H](Cc2cnc[nH]2)C(=O)N[C@H](C(=O)N[C@@H](CCC(N)=O)C(=O)N[C@@H](CCCNC(=N)N)C(=O)N[C@H](C(=O)N[C@@H](CCSC)C(N)=O)C(C)C)C(C)C)NC(=O)[C@H](CC(=O)O)NC(=O)[C@H](CC(C)C)NC1=O]

In some forms, the salts of Formulas I, II, III, III′ and III″ can be prepared by treating the free acid form of the compounds with an appropriate amount of a base. Exemplary bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

1. Amino Acids, Peptides, and Proteins

Peptides and polypeptides, such as multimers, oligomers, and polymers of or comprising amino acids, can be included in the disclosed compounds. For example, Q, L′, M′, and combinations thereof can be, comprise, or include peptides and polypetides.

The terms “polypeptide” and “protein” are used interchangeably herein to refer to an amino acid sequence comprising a polymer of amino acid residues. The term “peptide” refers to an amino acid sequence comprising an oligomer of amino acid residues. The terms also apply to amino acid polymers and oligomers in which one or more amino acid residues is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids, and isomers thereof. Naturally occurring amino acids are those encoded by the genetic code, as well as those amino acids that are later modified, e.g., hydroxyproline, carboxyglutamate, 0-phosphoserine, and isomers thereof. The term “amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The term “artificial amino acid” as used herein refers to an amino acid that is different from the twenty naturally occurring amino acids (alanine, arginine, glycine, asparagine, aspartic acid, cysteine, glutamine, glutamic acid, serine, threonine, histidine, lysine, methionine, proline, valine, isoleucine, leucine, tyrosine, tryptophan, phenylalanine) in its side chain functionality. The non-natural amino acid can be a close analog of one of the twenty natural amino acids, or it can introduce a completely new functionality and chemistry, as long as the hydrophobicity of the non-natural amino acid is either equivalent to or greater than that of the natural amino acid. The non-natural amino acid can either replace an existing amino acid in a protein (substitution), or be an addition to the wild type sequence (insertion). The incorporation of non-natural amino acids can be accomplished by known chemical methods including solid-phase peptide synthesis or native chemical ligation, or by biological methods. In certain embodiments, artificial amino acids include 4-fluoro-L-phenylalanine (F-Phe) and 1-methyl-L-tryptophan (Me-Trp).

2. Nucleic Acids

Nucleic acids, such as multimers, oligomers, and polymers of or comprising nucleotides, can be included in the disclosed compounds. For example, Q, L′, M′, and combinations thereof can be, comprise, or include nucleic acids.

Nucleic acids are multimers, oligomers, and polymers of nucleotides. The term “polynucleotide” refers to a nucleotide sequence comprising a polymer of nucleotide residues. The term “nucleotide oligomer” refers to a nucleotide sequence comprising an oligomer of nucleotide residues. The terms also apply to nucleotide polymers and oligomers in which one or more nucleotide residues is an artificial chemical mimetic of a corresponding naturally occurring amino acid, as well as to naturally occurring amino acid polymers and non-naturally occurring amino acid polymers.

As used herein, the term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a standard nucleotide can be adenin-9-yl (A), cytosin-1-yl (C), guanin-9-yl (G), uracil-1-yl (U), and thymin-1-yl (T). The sugar moiety of a standard nucleotide is a ribose or a deoxyribose. The phosphate moiety of a standard nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

As used herein, the term “nucleotide analog” refers to a nucleotide which contains some type of modification to the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

As used herein, the term “nucleotide substitute” refers to a nucleotide molecule having similar functional properties to nucleotides, but which does not contain a phosphate moiety. An exemplary nucleotide substitute is peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Unless the context indicates otherwise, reference to “nucleotide” herein refers to any of the forms of nucleotide (standard nucleotides, nucleotide analogs, and nucleotide substitutes).

In some forms, nucleic acids can comprise ribonucleotides and non-ribonucleotides. In some such forms, nucleic acids can comprise one or more ribonucleotides and one or more deoxyribonucleotides. In some forms, nucleic acids can comprise one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, peptide nucleic acids (PNA), bridged nucleic acids (BNA), or morpholinos. Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, peptide nucleic acid (PNA), morpholino, polyethylene glycol (PEG), triethylene glycol, or tetraethyleneglycol (TEG). Further examples of modified bases include, but are not limited to, 2-aminopurine, 5-bromo-uridine, pseudouridine (Ψ), N¹-methylpseudouridine (me¹Ψ), 5-methoxyuridine (5moU), inosine, 7-methylguanosine. Examples of nucleic acid chemical modifications include, without limitation, incorporation of 2′-O-methyl (M), 2′-O-methyl-3′-phosphorothioate (MS), phosphorothioate (PS), S-constrained ethyl (cEt), 2′-O-methyl-3′-thioPACE (MSP), or 2′-O-methyl-3′-phosphonoacetate (MP) at one or more terminal nucleotides.

Examples of modified nucleotides (such as non-naturally occurring nucleotides) include, but are not limited to, diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Nucleic acid molecules can also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Locked nucleic acid (LNA) is a family of conformationally locked nucleotide analogues which provide very high affinity and very high nuclease resistance to DNA and RNA oligonucleotides (Wahlestedt C, et al., Proc. Nat Acad. Sci. USA, 975633-5638 (2000); Braasch, D A, et al., Chem. Biol. 81-7 (2001); Kurreck J, et al., Nucleic Acids Res. 301911-1918 (2002)).

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen P E et al., Science 254, 1497-1500 (1991)). It is chemically stable and resistant to hydrolytic (enzymatic) cleavage.

In some forms, nucleic acid can comprise morpholino oligonucleotides. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

### III. Methods of Making the Compounds

Disclosed are methods of making the disclosed cyclic compounds. In some forms, methods of making the compounds of Formula I, Formula II, Formula III, Formula III′ and Formula III″ can involve:

(a) performing a reaction between a compound of Formula IV and a compound of Formula V.

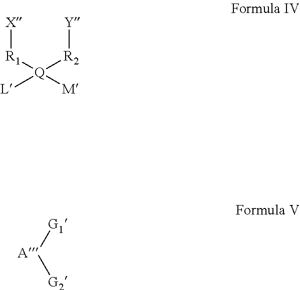

where R₁, R₂, Q, L′, and M′ are as defined above;

where X″ and Y″ are independently a carboxylic acid group, a carboxylate group,

an amino group optionally containing one substituent at the amino nitrogen, where the substituent is a substituted alkyl group, a substituted cycloalkyl group, a substituted heteroalkyl group, a substituted alkenyl group, a substituted heteroalkenyl group, a substituted aryl group, or a substituted heteroaryl group,

a hydroxyl group optionally containing one substituent at the hydroxyl oxygen, where the substituent is a substituted alkyl group, a substituted cycloalkyl group, a substituted heteroalkyl group, a substituted alkenyl group, a substituted heteroalkenyl group, a substituted aryl group, or a substituted heteroaryl group, or

a thiol group optionally containing one substituent at the thiol sulfur, where the substituent is a substituted alkyl group, a substituted cycloalkyl group, a substituted heteroalkyl group, a substituted alkenyl group, a substituted heteroalkenyl group, a substituted aryl group, or a substituted heteroaryl group;

where A′″ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; and

where G₁′ and G₂′ are reactive groups.

In some forms, X″ and Y″ can each be independently an amino group optionally containing one substituent at the amino nitrogen, where the substituent is a substituted alkyl group, a substituted cycloalkyl group, a substituted heteroalkyl group, a substituted alkenyl group, a substituted heteroalkenyl group, a substituted aryl group, or a substituted heteroaryl group, or a thiol group optionally containing one substituent at the thiol sulfur, where the substituent is a substituted alkyl group, a substituted cycloalkyl group, a substituted heteroalkyl group, a substituted alkenyl group, a substituted heteroalkenyl group, a substituted aryl group, or a substituted heteroaryl group.

In some forms, X″ and Y″ can each be independently an amine group or a thiol group. In some forms, X″ and Y″ are different and can each be independently an amino group or a thiol group. In some forms, X″ is a thiol group and Y″ is an amino group. In some forms, X″ is a thiol group and Y″ is an amine group.

In some forms, A′″ can be an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group. In some forms, A′″ can be an unsubstituted aryl group, a substituted aryl group, an unsubstituted polyaryl group, or a substituted polyaryl group. In some forms, A′″ can be an unsubstituted aryl group or a substituted aryl group.

In some forms, G1′ and G2′ can each be independently an aldehyde group, a cyanate group, a nitrile group, an isonitrile group, a nitro group, a nitroso group, a nitrosooxy group, an acyl group, a carboxylic acid group, or a carboxylate group. In some forms, G₁′ and G₂′ can each be independently an aldehyde group or an acyl group. In some forms, G₁′ and G₂′ are the same and can be aldehyde groups or acyl groups. In some forms, G₁′ and G₂′ are the same and can be aldehyde groups.

In a particular form, the compound of Formula V is ortho-phthalaldehyde (OPA). In another particular form, the compound of Formula V is 2,3-Thiophenedicarboxaldehyde (TDA).

In some forms, the compounds of Formula I, Formula II, Formula III, Formula III′ and Formula III″ can be made by:

(a) performing a reaction between a compound of Formula IV and a compound of Formula V to form an adduct, where Formula IV and Formula V are as defined above; and

(b) performing a reaction between the adduct from step (a) and a reactant to form a second adduct.

In some forms, the reactant can be an unsubstituted maleimide, a substituted maleimide, an unsubstituted alkynyl group, a substituted alkynyl group, or derivatives thereof. In some forms, the reactant can be an unsubstituted maleimide, a substituted maleimide, or derivatives thereof. In some forms, the reactant can be a maleimide derivative. In some forms, the reactant can be an unsubstituted alkynyl group, a substituted alkynyl group, or derivatives thereof. In some forms, the reactant can be a derivatized alkynyl group.

In some forms, the reaction of step (a) can be performed in a buffer solution. In some forms, the reaction of step (b) can be performed in a buffer solution. In some forms, the reactions of step (a) and step (b) can each be performed independently in a buffer solution. In some forms, the reactions of step (a) and step (b) can be performed in the same buffer solution. In some forms, the buffer solution can be acetate buffer, phosphate buffer, HEPES buffer, TEAA buffer, or borate buffer. In some forms, the reaction of step (a) can be performed at a pH of at least about 6, preferably at least about 7, more preferably at least about 7.4. In some forms, the reaction of step (a) can be performed at a pH of at least about 7. In some forms, the reaction of step (a) can be performed at a pH of at least about 7.4. In some forms, the reaction of step (a) can be performed at a pH between about 6 and about 10, between about 6.5 and about 10, between about 6.8 and about 10, between about 7 and about 10, between about 7.4 and about 10, or between about 8 and about 10.

In some forms, the reaction of step (b) can be performed at a pH of at least about 6, preferably at least about 7, more preferably at least about 7.4. In some forms, the reaction of step (b) can be performed at a pH of at least about 7. In some forms, the reaction of step (b) can be performed at a pH of at least about 7.4. In some forms, the reaction of step (b) can be performed at a pH between about 6 and about 10, between about 6.5 and about 10, between about 6.8 and about 10, between about 7 and about 10, between about 7.4 and about 10, or between about 8 and about 10.

In some forms, the reaction of step (a) is performed at a pH different from the reaction of step (b). In some forms, the reaction of step (a) is performed at the same pH as the reaction of step (b). In some forms, the reactions of both step (a) and step (b) are performed at a pH of at least about 7.4.

In some forms, the reaction of step (a) can be performed at room temperature. In some forms, the reaction of step (b) can be performed at room temperature. In some forms, the reactions of step (a) and step (b) can both be performed at room temperature.

In some forms, the reaction of step (a) can be performed at a rate where 80% of the compound of Formula IV and/or of the compound of Formula V has reacted at about 2.5 hours, preferably at about 2 hours, more preferably at about 1.5 hours. In some forms, the reaction of step (a) can be performed at a rate where 80% of the compound of Formula IV has reacted at about 2.5 hours, preferably at about 2 hours, more preferably at about 1.5 hours. In some forms, the reaction of step (a) can be performed at a rate where 80% of the compound of Formula V has reacted at about 2.5 hours, preferably at about 2 hours, more preferably at about 1.5 hours.

In some forms, the reaction of step (a) can be performed at a rate where 80% of the compound of Formula IV and/or of the compound of Formula V has reacted at about 30 minutes, preferably at about 20 minutes, more preferably at about 10 minutes. In some forms, the reaction of step (a) can be performed at a rate where 80% of the compound of Formula IV has reacted at about 30 minutes, preferably at about 20 minutes, more preferably at about 10 minutes. In some forms, the reaction of step (a) can be performed at a rate where 80% of the compound of Formula V has reacted at about 30 minutes, preferably at about 20 minutes, more preferably at about 10 minutes.

In some forms, the reaction of step (b) can be performed at a rate where 80% of the adduct formed in step (a) and/or the reactant has reacted at about 30 minutes, preferably at about 20 minutes, more preferably at about 10 minutes. In some forms, the reaction of step (b) can be performed at a rate where 80% of the adduct formed in step (a) has reacted at about 30 minutes, preferably at about 20 minutes, more preferably at about 10 minutes. In some forms, the reaction of step (b) can be performed at a rate where 80% of the reactant has reacted at about 30 minutes, preferably at about 20 minutes, more preferably at about 10 minutes.

In some forms, the reaction of step (a) can be performed at a rate where at least 80% of the compound of Formula IV and/or of the compound of Formula V has reacted at about 2.5 hours, preferably at about 2 hours, more preferably at about 1.5 hours. In some forms, the reaction of step (a) can be performed at a rate where at least 80% of the compound of Formula IV has reacted at about 2.5 hours, preferably at about 2 hours, more preferably at about 1.5 hours. In some forms, the reaction of step (a) can be performed at a rate where at least 80% of the compound of Formula V has reacted at about 2.5 hours, preferably at about 2 hours, more preferably at about 1.5 hours.

In some forms, the reaction of step (a) can be performed at a rate where at least 80% of the compound of Formula IV and/or of the compound of Formula V has reacted at about 30 minutes, preferably at about 20 minutes, more preferably at about 10 minutes. In some forms, the reaction of step (a) can be performed at a rate where at least 80% of the compound of Formula IV has reacted at about 30 minutes, preferably at about 20 minutes, more preferably at about 10 minutes. In some forms, the reaction of step (a) can be performed at a rate where at least 80% of the compound of Formula V has reacted at about 30 minutes, preferably at about 20 minutes, more preferably at about 10 minutes.

In some forms, the reaction of step (b) can be performed at a rate where at least 80% of the adduct formed in step (a) and/or the reactant has reacted at about 30 minutes, preferably at about 20 minutes, more preferably at about 10 minutes. In some forms, the reaction of step (b) can be performed at a rate where at least 80% of the adduct formed in step (a) has reacted at about 30 minutes, preferably at about 20 minutes, more preferably at about 10 minutes. In some forms, the reaction of step (b) can be performed at a rate where at least 80% of the reactant has reacted at about 30 minutes, preferably at about 20 minutes, more preferably at about 10 minutes.

In some forms, the reaction of step (a) can be performed at a rate different from that of step (b). In some forms, the reaction of step (a) can be performed at a rate the same as that of step (b).

In some forms, the reaction of step (a) can reach a conversion of at least about 70%, preferably at least about 80%, more preferably at least about 90%. In some forms, the reaction of step (a) can reach a conversion of at least about 80%. In some forms, the reaction of step (a) can reach a conversion of at least about 90%.

In some forms, the reaction of step (b) can reach a conversion of at least about 70%, preferably at least about 80%, more preferably at least about 90%. In some forms, the reaction of step (b) can reach a conversion of at least about 80%. In some forms, the reaction of step (a) can reach a conversion of at least about 90%.

In some forms, the conversion reached by the reaction of step (a) is different from that by the reaction of step (b). In some forms, the conversion reached by the reaction of step (a) is higher than that by the reaction of step (b). In some forms, the conversion reached by the reaction of step (a) is lower than that by the reaction of step (b).

In some forms, a purification step can be optionally performed following the reaction of step (a) and/or the reaction of step (b). In some forms, a purification step can be optionally performed following the reaction of step (a). In some forms, a purification step can be optionally performed following the reaction of step (b).

In some forms, the reaction of step (a) can be an OPA-cyclization reaction. This method allows for rapid and clean transformation, operational simplicity, moderate reaction conditions, and various post-modifications to achieve diverse functionalities. In some forms, the reaction of step (a) can be an OPA-cyclization reaction to form cyclo-peptide. In some forms, the cyclo-peptide can be a side chain-to-tail cyclo-peptide or a side chain-to-side chain cyclo-peptide. In some forms, the reaction is performed between an unprotected peptide and OPA. In some forms, the unprotected peptide can contain at least one lysine and at least one cysteine. In some forms, the lysine, cysteine, and OPA react to form a cyclo-peptide. In some forms, the lysine and cysteine can have a ratio of 1:1 mol/mol. Exemplary OPA-cyclization reaction to form cyclo-peptide is shown below:

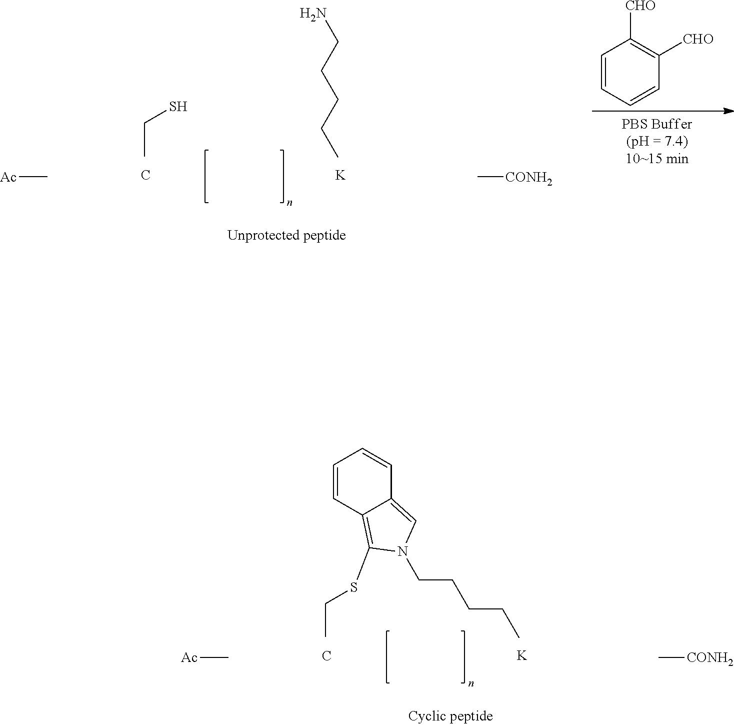

In some forms, the reaction of step (a) can be an TDA-cyclization reaction. This method allows for rapid and clean transformation, operational simplicity, moderate reaction conditions, and various post-modifications to achieve diverse functionalities. In some forms, the reaction of step (a) can be a TDA-cyclization reaction to form cyclo-peptide. In some forms, the cyclo-peptide can be a side chain-to-side chain cyclo-peptide. In some forms, the reaction is performed between an unprotected peptide and TDA. In some forms, the unprotected peptide can contain at least one lysine and at least one cysteine. In some forms, the lysine, cysteine, and TDA react to form a cyclo-peptide. In some forms, the lysine and cysteine can have a ratio of 1:1 mol/mol. Exemplary TDA-cyclization reaction to form cyclo-peptide is shown below:

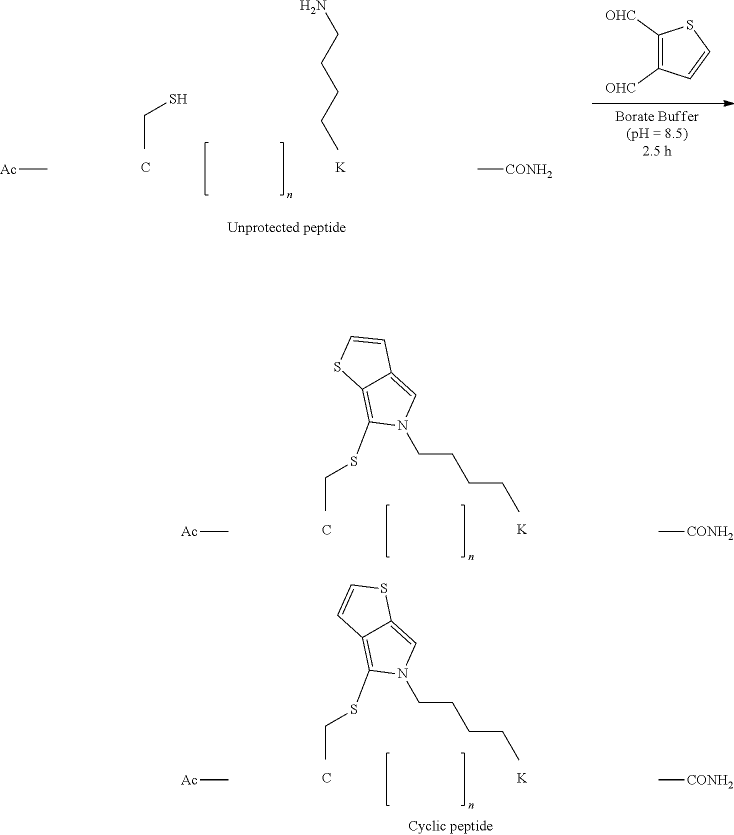

In some forms, the reaction of step (a) can be an OPA-cyclization reaction to form bicyclo-peptide. In some forms, the reaction can be performed between a cyclo-peptide and OPA. In some forms, the cyclo-peptide can contain at least one lysine and at least one cysteine. In some forms, the OPA-cyclization can be a side chain-to-tail reaction or a side chain-to-side chain reaction between lysine, cysteine, and OPA. In some forms, the lysine and cysteine can have a ratio of 1:1 mol/mol. In some forms, a NCL reaction can be performed prior to OPA-cyclization to provide a cyclo-peptide. Exemplary OPA-cyclization reaction to form bicyclo-peptide is shown below:

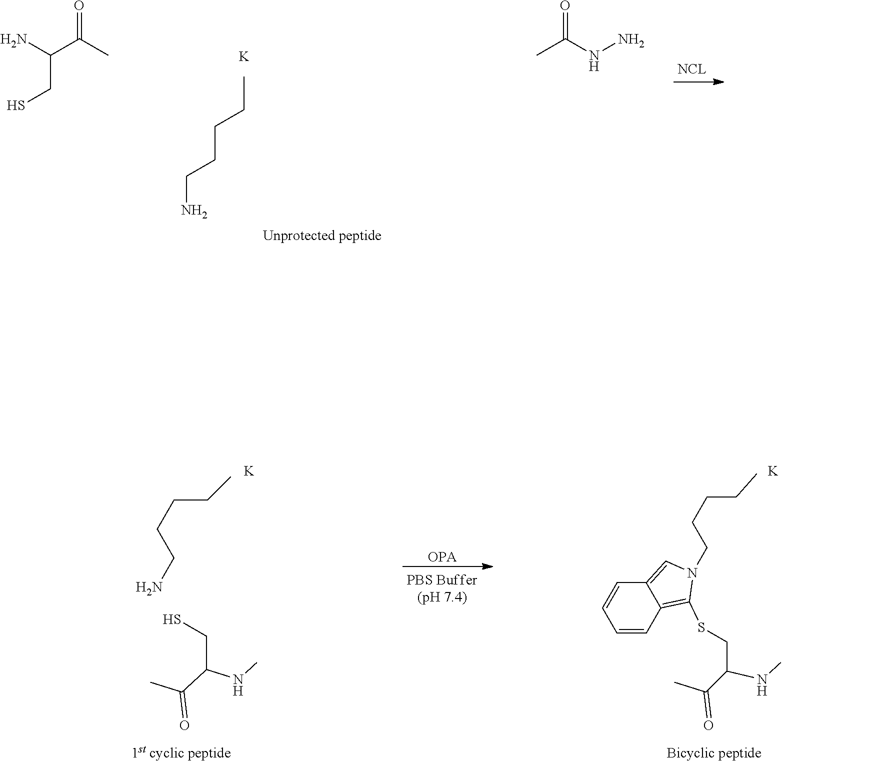

In some forms, post-modifications (step (b)) can be performed following an OPA-cyclization reaction to further modify cyclo-peptide and/or bicycle-peptide. In some forms, the adduct formed in the reaction of step (a) can be further modified with a reactant to increase the stability of the cyclo-peptide and/or bicycle-peptide, to introduce functionalities, or a combination of both.

In some forms, post-modifications (step (b)) can be performed following a TDA-cyclization reaction to further modify cyclo-peptide and/or bicycle-peptide. In some forms, the adduct formed in the reaction of step (a) can be further modified with a reactant to increase the stability of the cyclo-peptide and/or bicycle-peptide, to introduce functionalities, or a combination of both.

In some forms, ortho-phthalaldehyde (OPA) and/or 2,3-Thiophenedicarboxaldehyde (TDA) in step (a) and reactant in step (b) can be added sequentially to the reaction mixture containing peptide or cyclo-peptide and a solvent. In some forms, OPA and/or 2,3-Thiophenedicarboxaldehyde (TDA) in step (a) and reactant in step (b) can be added simultaneously to the reaction mixture containing peptide or cyclo-peptide and a solvent. In some forms, the solvent can be a buffer, an organic solvent, or a mixture of both. In some forms, the solvent can be a buffer. In some forms, the solvent can be an organic solvent. In some forms, the solvent can be a mixture of buffer and organic solvent. In some forms, the organic solvent can be dimethyl sulfoxide, methanol, ethanol, propanol, acetonitrile, ethylamine, or dimethylformamide. In some forms, the organic solvent can be dimethyl sulfoxide.

In some forms, the reactant can be dimethyl acetylenedicarboxylate (DMAC), N-maleimide, or maleimide derivatives. In some forms, the maleimide derivatives can contain a chemical probe or a biofunctional molecule. In some forms, the maleimide derivatives can contain a fluorophore, a peptide, an oligonucleotide, or a glycan. In some forms, the maleimide derivatives can contain a fluorophore. In some forms, the maleimide derivatives can contain a peptide. In some forms, the maleimide derivatives can an oligonucleotide. In some forms, the maleimide derivatives can contain a glycan.

Exemplary DMAC mediated post-modification of OPA-cyclization reaction is shown below:

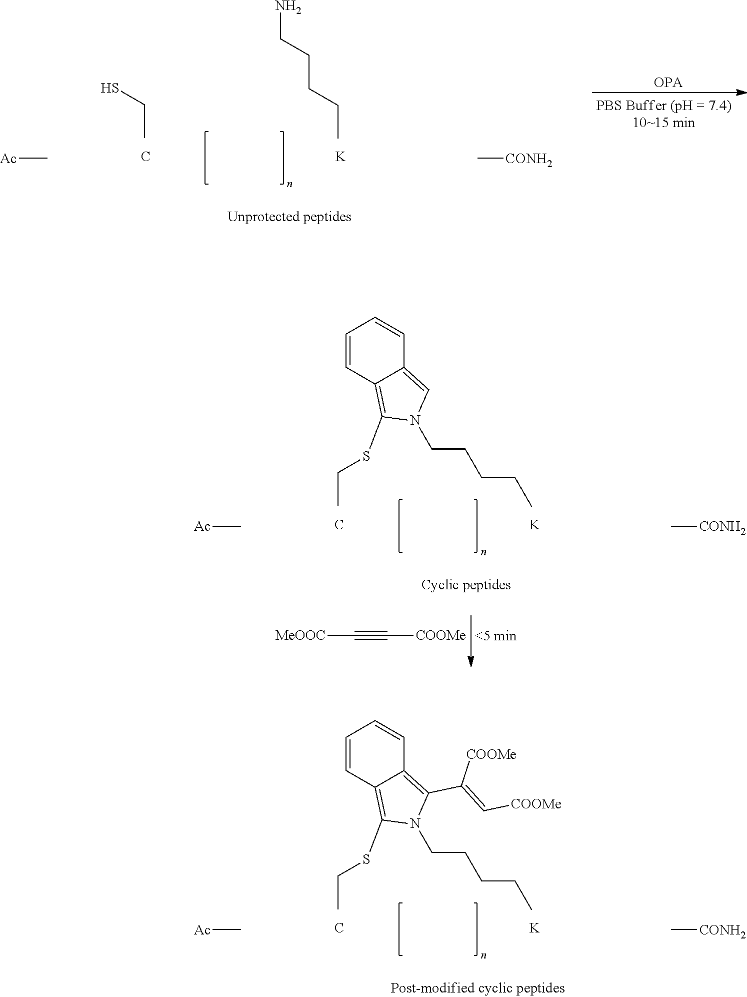

Exemplary fluorophore-maleimide mediated post-modification of OPA cyclization is shown below:

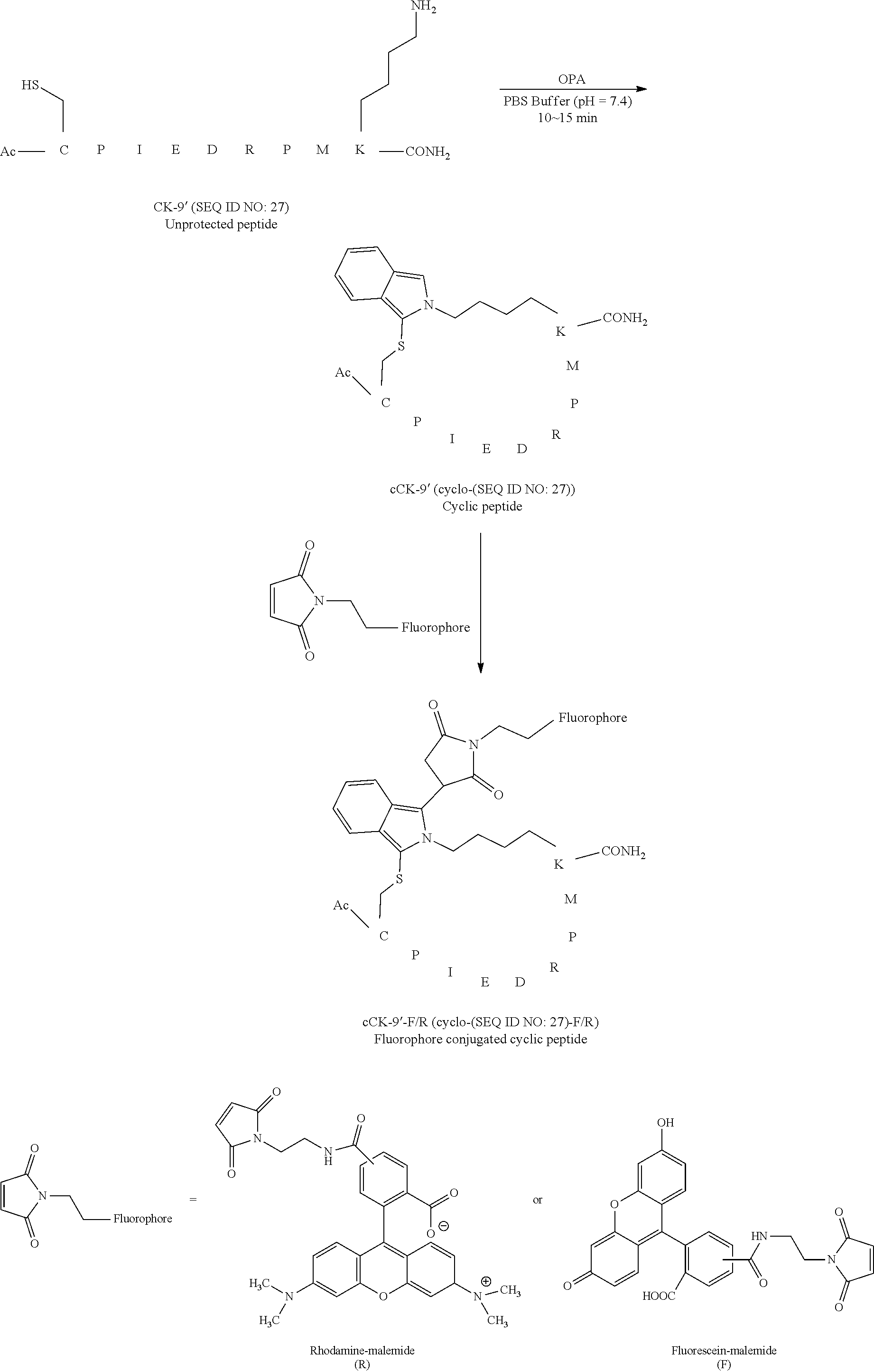

Exemplary biofunctional molecule-maleimide mediated post-modification of OPA cyclization is shown below:

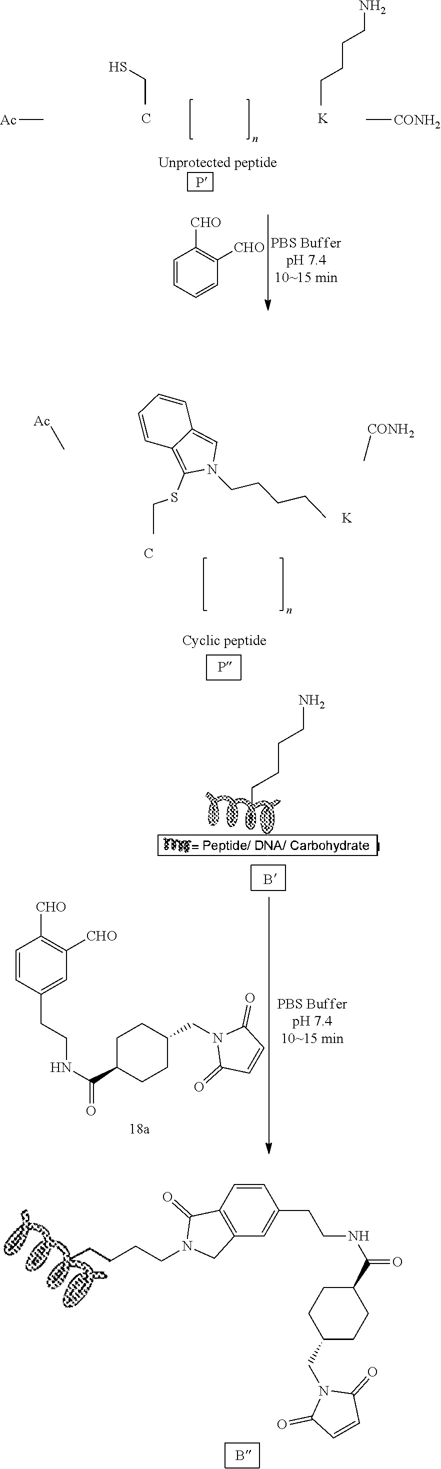

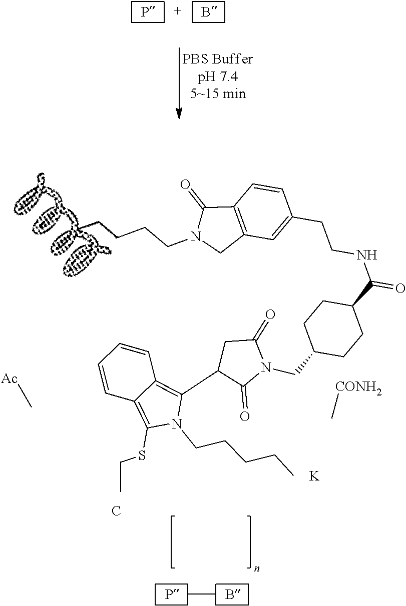

Exemplary methods to synthesize the specific compounds of Formula I, Formula II, Formula III, Formula III′ and Formula III″, i.e., for making 1a-1j, 7a-7c, 9a-9n, cKC10′-F, cKC10′-R, cKC9′-F, cKC9′-R, cCK9′-F, cCK9′-R, 20a-20e, 23a-23r, and 23a′-23r′ are described in the disclosed Examples.

### IV. Kits

The compounds described above can be packaged together with other components in any suitable combination as a kit useful for performing, or aiding in the performance of, the disclosed methods. It is useful if the components in a given kit are designed and adapted for use together in the disclosed methods.

In one aspect disclosed are kits for performing reactions for synthesis of cyclic compounds. The kits contains, in one or more containers, one or more of the disclosed compounds of Formula IV and Formula V, optionally one or more of the disclosed reactants, one or more buffers, as well as one or more other components, such as compounds, solvents, reactants, and carriers, instructions for use, and, optionally, an ionic or non-ionic detergent. The other components do not interfere with the effectiveness of the disclosed compounds of Formula IV and Formula V in the reactions for synthesis of cyclic compounds.

The kits can also contain an ionic or non-ionic detergent. The kits can also include instructions to use.

### V. Methods of Using the Compounds

One of the various forms of the disclosed cyclic compounds is a method of generating a library of cyclic compounds for drug discovery, i.e. construction of DNA-encoded cyclo-peptide library and phage-display cyclo-peptide library. In some forms, the disclosed cyclic compounds can be used in chemical biology study, i.e. cell imaging. The cyclic compounds can be used for both in vitro and in vivo chemical biology studies. In some forms, the disclosed cyclic compounds can be used as an in vitro probe for tissue staining and imaging and/or cell staining and imaging. In some forms, the disclosed cyclic compounds can be used as an in vivo probe for imaging. In some forms, the disclosed cyclic compounds can be used as a drug. In some forms, the disclosed cyclic compounds can be used for drug delivery, preferably targeted drug delivery. In some forms, the disclosed cyclic compounds can be used for high-throughput drug screening for the development of antibacterial cyclic peptides.

The disclosed compounds and methods can be further understood through the following numbered paragraphs.

1. A compound having a structure of Formula I:

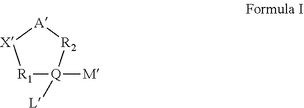

(a) wherein A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;

(b) wherein X′ is —NR₃, an oxygen atom, or a sulfur atom, wherein R₃ is a hydrogen, a substituted alkyl group, a substituted cycloalkyl group, a substituted heteroalkyl group, a substituted alkenyl group, a substituted heteroalkenyl group, a substituted aryl group, or a substituted heteroaryl group;

(c) wherein R₁ and R₂ are independently absent, an unsubstituted alkyl group, a substituted alkyl group, an unsubstituted heteroalkyl group, or a substituted heteroalkyl group;

(d) wherein Q is an oligomer or a synthetic material; and

(e) wherein L′ and M′ are independently absent, one or more monomer residues or a synthetic material.

2. The compound of paragraph 1, wherein the monomer residues are independently amino acid residues or nucleotide residues.

3. The compound of paragraph 1 or 2, wherein Q is a peptide or an oligonucleotide.

4. The compound of any one of paragraphs 1-3 having a structure of Formula II:

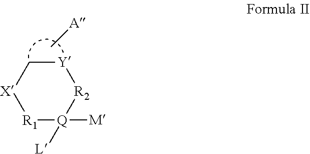

(a) wherein X′, R₁, R₂, Q, L′, and M′ are as defined in the base paragraph(s);

(b) wherein A″ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;

(c) wherein Y′ is a nitrogen atom.

5. The compound of any one of paragraph 4, wherein A″ is an unsubstituted polyaryl group, a substituted polyaryl group, an unsubstituted polyheteroaryl group, or a substituted polyheteroaryl group.

6. The compound of any one of paragraphs 1-5, wherein X′ is a sulfur atom.

7. The compound of any one of paragraphs 1-6, wherein Q is a peptide.

8. The compound of paragraph 7, wherein the peptide is a linear peptide, a cyclic peptide, or a branched peptide.

9. The compound of any one of paragraphs 1-8, wherein Q is an unprotected peptide.

10. The compound of any one of paragraphs 1-9, wherein L′ and M′ are independently one or more amino acid residues.

11. The compound of any one of paragraphs 1-6, wherein Q is an oligomer of synthetic monomer residues.

12. The compound of any one of paragraphs 1-11, wherein the compound is fluorescent.

13. The compound of any one of paragraphs 1-12 having a structure of Formula III:

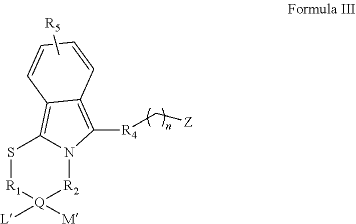

(a) wherein R₁, R₂, Q, L′, and M′ are as defined in the base paragraph(s);

(b) wherein R₄ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

(c) wherein R₅ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group, an unsubstituted carbonyl group, a substituted carbonyl group,

an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,

an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or

a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;

(d) wherein n is zero or a positive integer; and

(e) wherein Z is optional and comprises a chemical probe and/or a biofunctional molecule.

14. The compound of any one of paragraphs 1-13, having a structure of Formula III′ or Formula III″

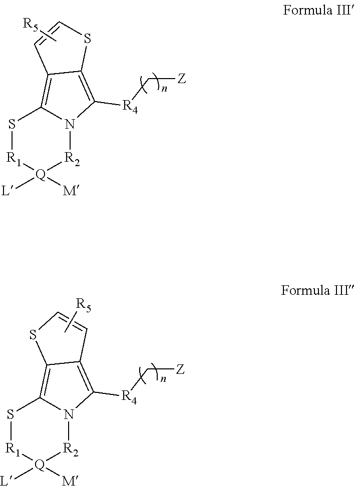

wherein R₁, R₂, R₄, R₅, Q, L′, M′, n and Z are as defined above.

15. The compound of paragraph 13 or paragraph 14, wherein when R₄ is hydrogen, n is zero, and Z is absent.

16. The compound of paragraph 13 or paragraph 14, wherein R₄ is an unsubstituted alkenyl group, a substituted alkenyl group, an unsubstituted heteroalkenyl group, or a substituted heteroalkenyl group.

17. The compound of paragraph 13 or paragraph 14, wherein R₄ is an unsubstituted succinimidyl group or a substituted succinimidyl group.

18. The compound of any one of paragraphs 13-17, wherein Z comprises a luminescence probe.

19. The compound of paragraph 18, wherein the luminescence probe is an organic dye, a biological fluorophore, or a quantum dot.

20. The compound of paragraph 19, wherein the luminescence probe is an organic dye selected from the group consisting of fluorescein, rhodamine, and derivatives thereof.

21. The compound of any one of paragraphs 13-20, wherein Z comprises a colorimetric probe.

22. The compound of any one of paragraphs 13-21, wherein Z comprises a biofunctional molecule selected from the group consisting of glycans, peptides, oligonucleotides, proteins, and small molecule drugs.

23. A method of making the compound of any one of paragraphs 1-22, comprising:

(a) performing a reaction between a compound of Formula IV and a compound of Formula V to form an adduct,

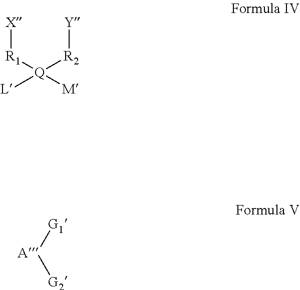

wherein R₁, R₂, Q, L′, and M′ are as defined in the base paragraph(s);

wherein X″ and Y″ are independently a carboxylic acid group, a carboxylate group,
an amino group optionally containing one substituent at the amino nitrogen, wherein the substituent is a substituted alkyl group, a substituted cycloalkyl group, a substituted heteroalkyl group, a substituted alkenyl group, a substituted heteroalkenyl group, a substituted aryl group, or a substituted heteroaryl group,
a hydroxyl group optionally containing one substituent at the hydroxyl oxygen, wherein the substituent is a substituted alkyl group, a substituted cycloalkyl group, a substituted heteroalkyl group, a substituted alkenyl group, a substituted heteroalkenyl group, a substituted aryl group, or a substituted heteroaryl group, or
a thiol group optionally containing one substituent at the thiol sulfur, wherein the substituent is a substituted alkyl group, a substituted cycloalkyl group, a substituted heteroalkyl group, a substituted alkenyl group, a substituted heteroalkenyl group, a substituted aryl group, or a substituted heteroaryl group;

wherein A′″ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; an

wherein G₁′ and G₂′ are independently an aldehyde group, a cyanate groups, a nitrile group, an isonitrile group, a nitro group, a nitroso group, a nitrosooxy group, an acyl group, a carboxylic acid group, or a carboxylate group.

24. The method of paragraph 23, further comprising:

(b) performing a reaction between the adduct from step (a) and a reactant to form a second adduct, wherein the reactant is an unsubstituted maleimide, a substituted maleimide, an unsubstituted alkynyl group, a substituted alkynyl group, or derivatives thereof.

25. The method of paragraph 23 or 24, wherein the compound of Formula V is ortho-phthalaldehyde (OPA).

26. The method of paragraph 23 or 24, wherein the compound of Formula V is 2,3-Thiophenedicarboxaldehyde (TDA).

27. The method of any one of paragraphs 23-26, wherein X″ is a thiol group and Y″ is an amino group.

28. The method of any one of paragraph 23-27, wherein the reaction is performed in a buffer solution.

29. The method of paragraph 28, wherein the buffer solution is selected from the group consisting of acetate buffer, phosphate buffer, HEPES buffer, TEAA buffer, and borate buffer.

30. The method of any one of paragraphs 23-29, wherein the reaction is performed at a pH of at least about 6, preferably at least about 7, more preferably at least about 7.4.

31. The method of any one of paragraphs 23-30, wherein the reaction is performed at a pH of at least about 8, preferably at least about 8.5.

32. The method of any one of paragraphs 23-31, wherein the reaction in step (a) is performed at a rate wherein at least 80% of the compound of Formula IV and/or of the compound of Formula V has reacted at about 2.5 hours, preferably at about 2 hours, more preferably at about 1.5 hours.

33. The method of any one of paragraphs 23-32, wherein the reaction in step (a) is performed at a rate wherein 80% of the compound of Formula IV and/or of the compound of Formula V has reacted at about 30 minutes, preferably at about 20 minutes, more preferably at about 10 minutes.

34. The method of any one of paragraphs 23-33, wherein the reaction in step (b) is performed at a rate wherein 80% of the adduct formed in step (a) and/or the reactant has reacted at about 30 minutes, preferably at about 20 minutes, more preferably at about 10 minutes.

35. The method of any one of paragraphs 23-34, wherein the reaction of step (a) reaches a conversion of at least about 70%, preferably at least about 80%, more preferably at least about 90%.

36. The method of any one of paragraphs 23-35, wherein the reaction of step (b) reaches a conversion of at least about 70%, preferably at least about 80%, more preferably at least about 90%.

The present invention will be further understood by reference to the following non-limiting examples.

### EXAMPLES

The Examples below demonstrated highly effective chemoselective peptide cyclization and bicyclization directly on unprotected peptides, which generates novel structural motif. The fast reaction rate and operational simplicity render this method to be highly effective to synthesize cyclic structures, i.e. cyclic peptides.

The base cyclization reaction produces a cyclized structure that can be used in further reactions. Both the base cyclization and the further reactions can be one-pot reactions, including the sequential combination of both reactions. The reactions are operationally simple, highly efficient (<30 minutes for two steps), and performed under physiological conditions. It is especially notable that the peptide to be cyclized does not need to have any protecting groups and that the reactions are highly chemoselective.

Overall, the OPA/TDA cyclization method can be applied for the synthesis of various functional cyclic/bicyclic peptides, peptide conjugates and branched peptides in both chemical biology study and drug discovery. The operational simplicity and high efficiency of OPA/TDA peptide cyclization will also potentially provide a new tool for construction of DNA-encoded cyclic peptide library.

### Example 1. OPA-Cyclization Provides a Simple Way to Cyclize Peptides

Materials and Methods

Peptide Synthesis

All commercial materials (Sigma-Aldrich, Acros Organics, J&K Scientific and GL Biochem) were used without further purification. All solvents were reagent grade or HPLC grade (RCI or DUKSAN). Dry dichloromethane (CH₂Cl₂) was distilled from calcium hydride (CaH₂). Analytical TLC was performed on Silica gel 60 F254 pre-coated glass plates. The normal phase column chromatography was performed on silica gel (230-400 mesh, Merck). 1H and ¹³C NMR spectra were recorded on Bruker Avance DRX 400 FT-NMR spectrometer at 400 MHz for 1H NMR and 100 MHz for ¹³C NMR or Bruker Avance DRX 500 FT-NMR spectrometer at 500 MHz for 1H NMR and 126 MHz for ³C NMR. HPLC and MALDI TOF MS methods were described specifically in the corresponding text below.

All commercial materials (Aldrich, CSBio, Chem-Impex and GL Biochem) were used without further purification. All solvents were reagent grade or HPLC grade (RCI or DUKSAN). The following Fmoc amino acids were purchased from GL Biochem and Chemimpex and used in the solid phase synthesis: Fmoc-Ala-OH, Fmoc-Asn(Trt)-OH, Fmoc-His(Trt)-OH, Fmoc-Ile-OH, Fmoc-Leu-OH, Fmoc-Val-OH, Fmoc-Lys(Boc)-OH, Fmoc-Phe-OH, Fmoc-Ser(tBu)-OH, Fmoc-Thr(tBu)-OH, Fmoc-Tyr(tBu)-OH, Fmoc-Glu(OtBu)-OH, Fmoc-Asp(OtBu)-OH, Fmoc-Gln(Trt)-OH, Fmoc-Pro-OH, Fmoc-Met-OH, Fmoc-Gly-OH, Fmoc-Arg(Pbf)-OH, Fmoc-Trp(Boc)-OH, Fmoc-Cys(Trt)-OH, Fmoc-Dap(Boc)-OH, Fmoc-Hyp(tBu)-OH, Fmoc-D-Thr(tBu)-OH. All separations involved a mobile phase of 0.1% TFA (v/v) in acetonitrile and 0.1% TFA (v/v) in water. HPLC separations were performed with Waters HPLC system equipped with photodiode array detector (Waters 2996) using Vydac 218TP C18 colmn (5 m, 300 Å, 4.6×250 mm) at a flow rate of 0.6 mL/min for analytical HPLC and XBrigdge Prep C18 10 μm OBD column (10 m, 300 Å, 30×250 mm) at a flow rate of 15 mL/min for preparative HPLC. Mass spectral analyses were performed with Water 3100 mass spectroeter.

RP-LCMS Detection

For all the RP-LCMS and RP-UPLC detection of OPA-peptide conjugates, all separations involved a mobile phase of 0.1% TFA (v/v) in acetonitrile and 0.1% TFA (v/v) in water. RP-LCMS analysis was performed with Waters HPLC system equipped with photodiode array detector (Waters 2996) using Vydac 218TP C18 column (5 μm, 300 Å, 4.6×250 mm) at a flow rate of 0.6 mL/min. Waters UPLC H-class system equipped with an ACQUITY UPLC photodiode array detector and a Waters SQ Detector 2 mass spectrometer using a Waters ACQUITY BEH C18 column (1.7 μm, 130 Å, 2.1×50 mm) at a flow rate of 0.4 mL/min.

Solid Phase Peptide Synthesis and Native Chemical Ligation

Synthesis was performed manually on rink amide resin (GL Biochem) under the standard Fmoc protocol. Removal of Fmoc protecting group was performed using a mixture of 20/80 (v/v) of piperidine/DMF for 15-20 min. Coupling was performed using Fmoc-Amino acids (4.0 equiv.), HATU (4.0 equiv.) and DIPEA (8.0 equiv.) in DMF for 1 hour at room temperature. For N-terminal acetylated peptide, anhydrous CH₂Cl₂: Pyridine: Acetic anhydride (2:1:1, v:v:v) with resin for 1 h at room temperature. Upon completion of the synthesis, 9.5:0.25:0.25 of TFA: TIPS: Water (v:v:v) were used to perform global deprotection. The peptides were then precipitated in cold diethyl ether and purified by preparative RP-HPLC.

Preparation of hydrazine 2-chlorotrityl Chloride Resin

The 2-chlorotrityl chloride resin (500 mg, loading=0.5 mmol/g) was placed in a 10 mL polypropylene syringe with a polyethylene filter in the bottom. Pre-swelling the resin in anhydrous DCM for 20 min. Then wash the resin by DCM (3×6 mL) and DMF (3×6 mL). The NH₂NH₂.H₂O/DMF solution (1/15, v/v, 6 mL) was added into the resin and the mixture was shaken for 1 hour. Then wash the resin by DCM (3×6 mL) and DMF (3×6 mL). Another portion of NH₂NH₂.H₂O/DMF solution (1/15, v/v, 6 mL) was added and the mixture was shaken for 30 min. The resin was then washed by DCM (3×8 mL) and DMF (3×6 mL) again. The remaining 2-chlorotrityl chloride resin was quenched by methanol/DMF solution (1/15, v/v, 6 mL) for 20 min. The resin was then washed by DMF (3×8 mL) and ready for Fmoc-Solid phase peptide synthesis (Zheng, et al., Nature protocols., 8(12):2483 (2013)).

OPA-Cyclization with Model Peptides

Model peptides (with a lysine and cysteine, 1 equiv.) were dissolved in phosphate buffered saline (PBS buffer) (pH=7.4) with a final concentration of 0.5 mM. Phthaldialdehyde (1.2 equiv.) was added to the solution and the reaction was stirred at room temperature around 10-15 min. The conversion was monitored by LC-MS.

Results

Scheme of the OPA-guided chemoselective cyclization:

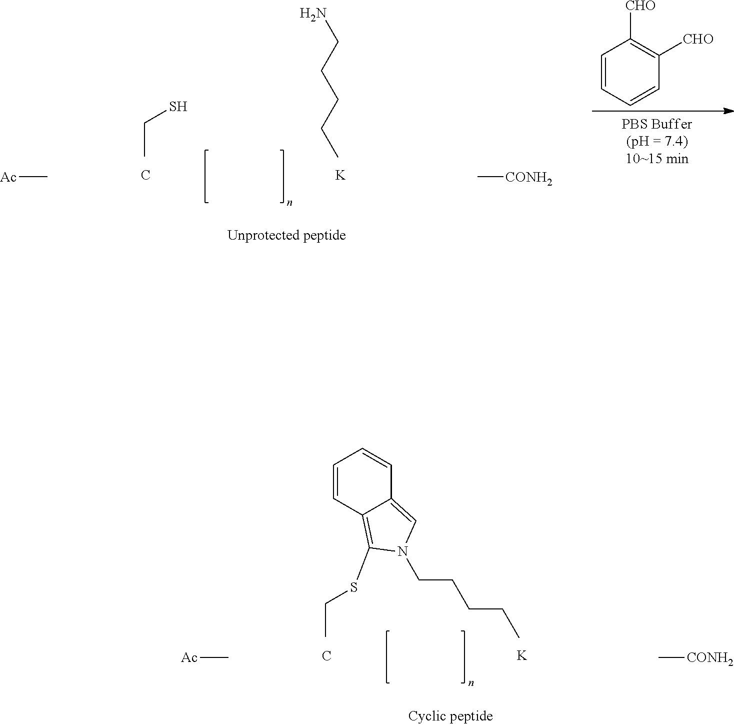

OPA can react with free amine groups rapidly in aqueous buffers to form phthalimidines (Zhang, et al., Org. Lett., 14(19):5146-5149 (2012); Tung, et al., Org. Lett., 18(11):2600-2603 (2016)). It was discovered that a two-component reaction of amine and OPA to form phthalimidines competes with the three-compound reaction of amine, OPA, and thiol to form isoindoles. For this reason, it is useful to use a large excess of thiol groups.

The model peptide (Ac-KAAAAAACF-CONH₂; SEQ ID NO:7) carried a cysteine residue and a lysine. Upon addition of OPA to the peptide in aqueous PBS buffer (pH 7.4), surprisingly, the reaction turned out to be very smooth and rapid to form the isoindole cyclic peptide, with full conversion within 10 minutes and without any trace of the two-component reaction product (c.f., phthalimidines) or other byproducts, judged by the LCMS analysis of the crude reaction mixture (See compounds 1a-1j described above). OPA was used in stoichiometric amount. This reaction is a simple and robust thiol-amine cyclization that is highly efficient and chemoselective. The expected intrinsic fluorescence was obtained after cyclization.

Ten model peptides with different length and spacing amino acids of 4 to 7 residues between the N-terminus or Lys side chain and the Cys residue reacted with OPA in the PBS buffer to afford cyclic peptides of different rings, with 93->98% conversions judged by LCMS analysis of the crude reaction mixtures (See compounds 1a-1j described above and Table 1). Various side chain functionalities present in the unprotect peptides did not interfere the reaction. Thus, this chemoselective OPA-cyclization provides a simple way to cyclize unprotected native peptides. It should be pointed out that this reaction couldn't differentiate the side chain amino group and the N-terminal amine, thus capable of producing both side chain-to-tail and side chain-to-side chain cyclic peptides. Orthogonal amine protecting groups need to be used when multiple lysine residues are present in the peptide sequence.

TABLE 1
OPA-guided cyclization with different peptide sequences.

| No. | Sequence | Cal. MS | Obtained MS | Conversion |
|---|---|---|---|---|
| 1a | NH₂-AAACF-CONH₂ | [M + H]⁺ = 578.20 | 579.2 | >98% |
| | (SEQ ID NO: 1) | | | |
| 1b | NH₂-AAAACF-CONH₂ | [M + H]⁺ = 649.66 | 650.3 | 96% |
| | (SEQ ID NO: 2) | | | |
| 1c | NH₂-AAAAACF-CONH₂ | [M + H]⁺ = 720.31 | 721.3 | >98% |
| | (SEQ ID NO: 3) | | | |
| 1d | Ac-KAAACF-CONH₂ | [M + H]⁺ = 748.30 | 749.4 | 98% |
| | (SEQ ID NO: 4) | | | |
| 1e | Ac-KAAAACF-CONH₂ | [M + H]⁺ = 819.30 | 820.2 | 93% |
| | (SEQ ID NO: 5) | | | |
| 1f | Ac-KAAAAACF-CONH₂ | [M + H]⁺ = 890.41 | 891.3 | >98% |
| | (SEQ ID NO: 6) | | | |
| 1g | Ac-KAAAAAACF-CONH₂ | [M + H]⁺ = 961.41 | 962.4 | >98% |
| | (SEQ ID NO: 7) | | | |
| 1h | Ac-CHHALTHAK-CONH₂ | [M + H]⁺ = 1155.50 | 1156.5 | >98% |
| | (SEQ ID NO: 8) | | | |
| 1i | Ac-CAHNLTHAK-CONH₂ | [M + H]⁺ = 1132.50 | 1133.5 | >98% |
| | (SEQ ID NO: 9) | | | |
| 1j | Ac-ENPECILDKHVQRVM- | [M + 2H]²⁺ = 976.02 | 975.9 | >98% |
| | CONH₂ (SEQ ID NO: 10) | | | |

### Example 2. Buffer Condition Affects OPA-Cyclization

Materials and Methods

Model peptide Ac-KAAACH-CONH₂ (SEQ ID NO: 16) (0.5 mM, 1 equiv.) was dissolved in various aqueous buffer solutions with a final concentration of 0.5 mM. OPA (1 equiv.) in DMSO was added into the reaction and stirred at room temperature for 15 min. After 15 min, the reaction was quenched by 10 μL of hydrazine monohydrate. Then the reaction was diluted by ACN/H₂O (with 0.1% TFA) and monitored the conversion by RP-UPLC and the conversion was calculated based on LC-MS spectrum.

Results

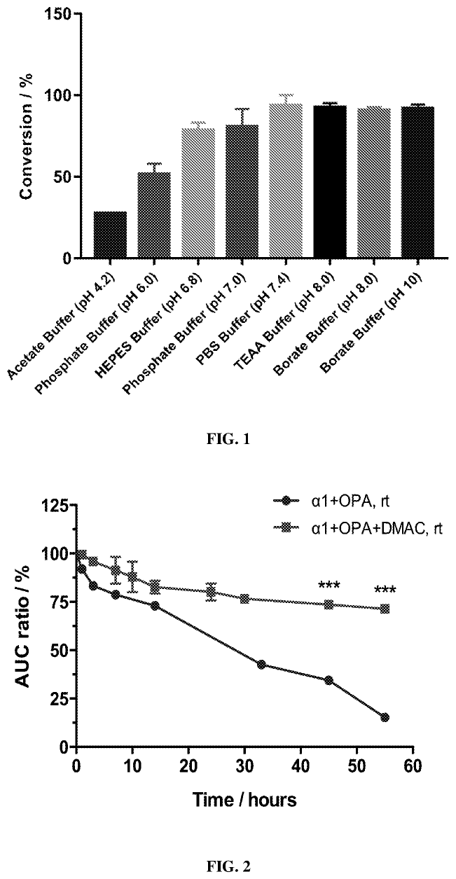

Further conditions screening showed that buffers with pH 7.4 or above gave very clean reaction, while buffers with pH 7 or below resulted in some minor unidentified byproduct, and pH 3 was not good at all (See FIG. 1).

### Example 3. Reaction of OPA and Intramolecular Thiol-Amine Offers Effective Peptide Cyclization

Materials and Methods

Model peptide Ac-ENPECILDKHVQRVM-CONH₂ (SEQ ID NO:10) (1 equiv.) and Ac-AFAQK-CONH₂ (SEQ ID NO:11) (1 equiv.) were dissolved in PBS buffer with a final concentration of 0.02 mM. OPA (1 equiv.) in DMSO was added into the mixture and stirred at room temperature for 30 min. After 30 min, the reaction solution was directly monitored by RP-UPLC and the conversion was calculated based on the LC-MS spectrum.

Results

The competition experiment was conducted to probe the reaction pathway. The peptide (Ac-AFAQ [symbol] -CONH₂) (SEQ ID NO:11) with only lysine and the peptide (Ac-ENPE [symbol] ILD [symbol] HVQRVM-CONH₂) (SEQ ID NO:10) with both lysine and cysteine were mixed in 1:1 ratio and reacted with OPA (1.0 equiv.) at a concentration of 0.02 mM in the PBS buffer. Both the two-component reaction product (6%) and three-component reaction product (94%) were observed from LCMS analysis (See Table 2). This result shows that after the imine formation, the intramolecular thiol attack on the imine was times faster than the hydroxyiminum formation, affording the three-component reaction product as the major product. It is also possible that the thiol reacted with the OPA to form a thiohemiacetal first, followed by the reaction with the intramolecular amine. In any scenario, the reaction of OPA and intramolecular thiol-amine offers a very effective peptide cyclization.

TABLE 2
The conversion of competition reaction

| Reagents | Equivalent | Conversion^{[a]} |
|---|---|---|
| OPA | 1 | — |
| Ac-AFAQK-CONH₂ (SEQ ID | 1 | 6.68 |
| NO: 11) | | |
| Ac-ENPECILDKHVQRVM-CONH₂ | 1 | 93.3 |
| (SEQ ID NO: 10) | | |

^{[a]}conversion percentage was calculated based on LCMS profile.

### Example 4. NCL- and OPA-Cyclization Provides Bicyclic Products with Over 90% Conversion

Materials and Methods

Scheme of the bicyclic peptides formation via NCL and OPA cyclization:

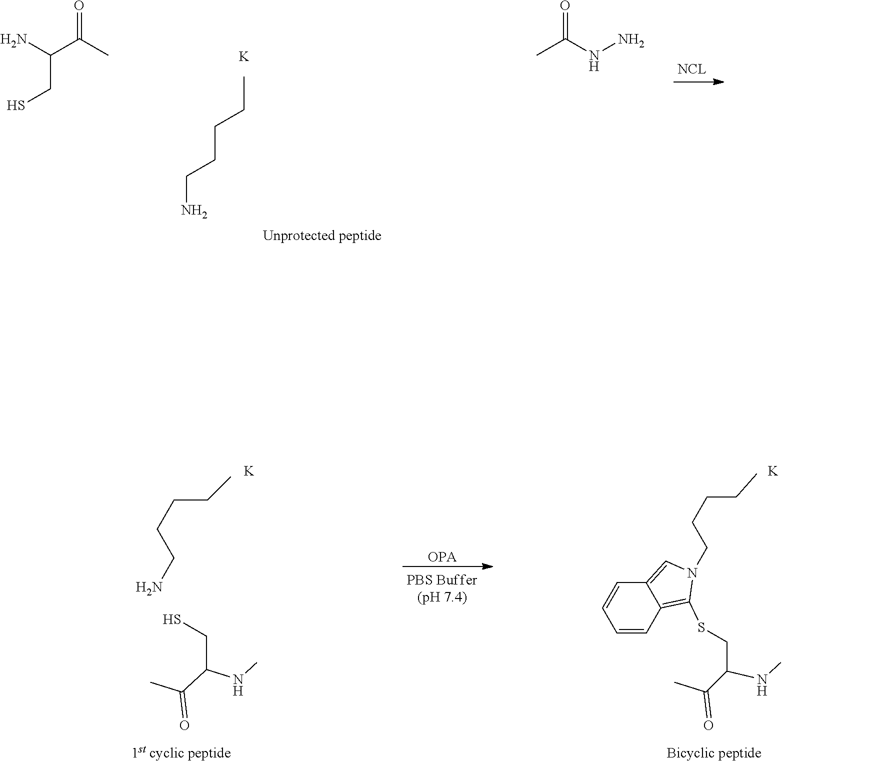

Bicyclo-(CSSLDEPGRGGFSSESKV) (SEQ ID NO:12) Scheme of bicyclo-(CSSLDEPGRGGFSSESKV) (7a) (SEQ ID NO:12) formation via NCL and OPA cyclization:

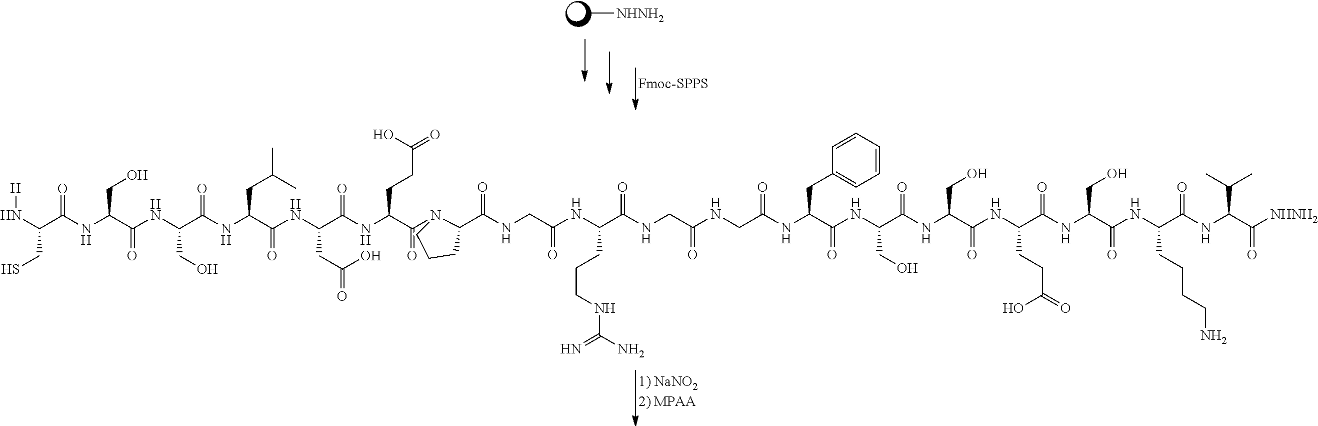

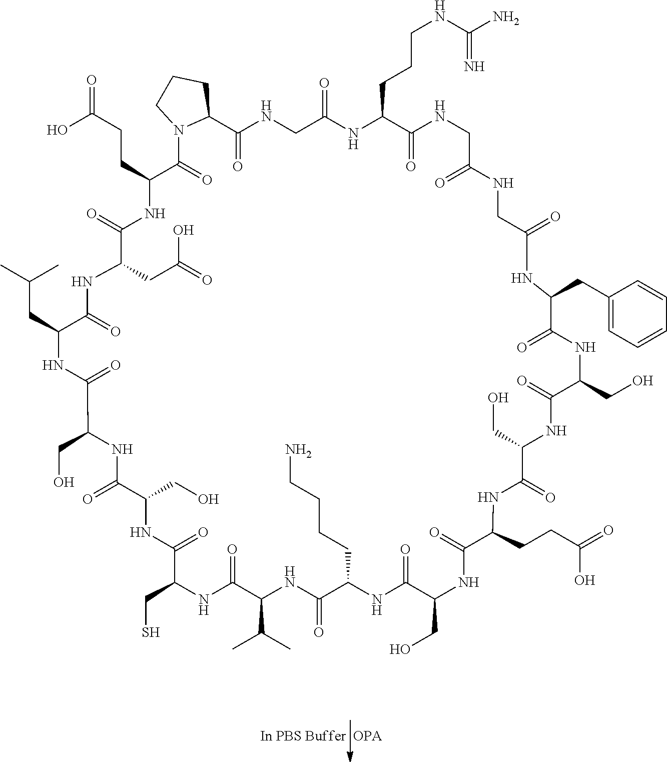 [SMILES: CC(C)C[C@@H]1NC(=O)[C@H](CO)NC(=O)[C@H](CO)NC(=O)[C@H](CS)NC(=O)[C@H](C(C)C)NC(=O)[C@H](CCCCN)NC(=O)[C@H](CO)NC(=O)[C@H](CCC(=O)O)NC(=O)[C@H](CO)NC(=O)[C@H](CO)NC(=O)[C@H](Cc2ccccc2)NC(=O)CNC(=O)CNC(=O)[C@H](CCCNC(=N)N)NC(=O)CNC(=O)[C@@H]2CCCN2C(=O)[C@H](CCC(=O)O)NC(=O)[C@H](CC(=O)O)NC1=O ; "OPA"]

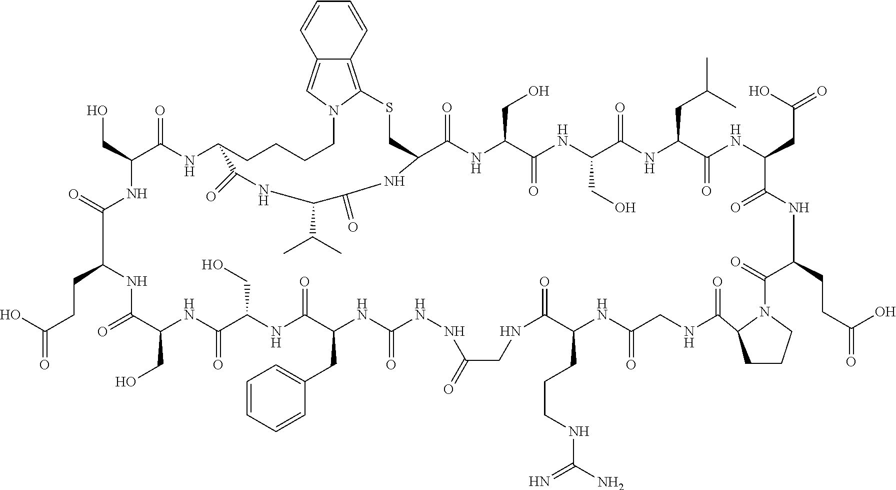 [SMILES: CC(C)C[C@@H]1NC(=O)[C@H](CO)NC(=O)[C@H](CO)NC(=O)[C@@H]2CSc3c4ccccc4cn3CCCC[C@H](NC(=O)[C@H](CO)NC(=O)[C@H](CCC(=O)O)NC(=O)[C@H](CO)NC(=O)[C@H](CO)NC(=O)[C@H](Cc3ccccc3)NC(=O)NNC(=O)CNC(=O)[C@H](CCCNC(=N)N)NC(=O)CNC(=O)[C@@H]3CCCN3C(=O)[C@H](CCC(=O)O)NC(=O)[C@H](CC(=O)O)NC1=O)C(=O)N[C@@H](C(C)C)C(=O)N2]

Peptide Synthesis

Peptide NH₂—CSSLDEPGRGGFSSESKV-CONHNH₂ (SEQ ID NO:34) was synthesized by the general SPPS procedure on the hydrazine 2-chlorotrityl chloride resin. Preparative HPLC purification (10%-60% ACN/H₂O with 0.1% TFA over 45 min), then concentrated under vacuum and lyophilization to NH₂-CSSLDEPGRGGFSSESKV-CONHNH₂ (SEQ ID NO:34) as a white powder.

NCL Buffer preparation (pH=3 and pH=7) NCL Buffer (pH=3.0): Na₂HPO₄ (284 mg, 2.0 mmol) and Gn.HCl (5.7 g, 60.0 mmol) was dissolved in 10 mL distilled water. After ultrasonic dissolved all the solids, the pH of the mixture was carefully adjusted to 3.0 (By using pH meter) with 1 M HCl solution and 1 M NaOH solution. NCL buffer (pH=7.0): Na₂HPO₄ (284 mg, 2.0 mmol) and Gn.HCl (5.7 g, 60.0 mmol) was dissolved in 10 mL distilled water. After ultrasonic dissolved all of the solids, the pH of the mixture was carefully adjusted to 7.0 (By pH meter) with 1 M HCl solution and 1 M NaOH solution. All the buffer solution was fresh prepared before using.

NCL-Cyclization

The peptide NH₂—CSSLDEPGRGGFSSESKV-CONHNH₂ (SEQ ID NO:34) (5.18 mg, 2.79 μmol) was dissolved in pH=3.0 NCL buffer (containing 6.0 mol/L Gn.HCl and 0.2 mol/L NaH₂PO₄, 20 mL) completely, then the reaction mixture was cooled down to −15° C. Then slowly add 0.2 M NaNO₂ solution (70 μL) into the reaction mixture. After the reaction was stirred at −15° C. for 15 min, the 0.2M MPAA solution (in NCL buffer, pH=7, 0.7 mL) was added into the reaction mixture. The pH value of the ligation mixture was carefully adjusted to 6.8 to 7.0 with 1.0 M NaOH solution, the reaction was allowed to warm up and stirred at room temperature for 5 h to 7 h. After the reaction was finished, 20.0 equiv. of TCEP.HCl (0.1 M) in pH=7.0 phosphate buffer was added to reduce the reaction. Then the reaction was monitored by UPLC. The reaction mixture would be diluted by ACN/H₂O before HPLC purification. Preparative HPLC purification (15%-60% ACN/H₂O with 0.1% TFA over 45 min) followed by concentration under vacuum and lyophilization afforded cyclo-(CSSLDEPGRGGFSSESKV) (SEQ ID NO:12) (3.5 mg, 1.9 μmol, 68.8%) as a white powder.

UV trace from LC-MS analysis of the crude NCL cyclic reaction mixture, cyclo-(CSSLDEPGRGGFSSESKV) (SEQ ID NO:12), shows a clean NCL cyclization reaction (gradient: 5%-95% ACN/H₂O with 0.1% TFA over 10 min at a flow rate of 0.4 mL/min). ESI-MS calcd. for C₇₅H₁₁₈N₂₂O₂₉S [M+H]⁺ m/z=1823.95, [M+2H]²⁺ m/z=912.97, found 913.19 (crude product).

UV trace from LC-MS analysis of cyclo-(CSSLDEPGRGGFSSESKV) (SEQ ID NO:12) after purification shows pure NCL-cyclized product (gradient: 5%-95% ACN/H₂O with 0.1% TFA over 5 min at a flow rate of 0.4 mL/min). ESI-MS calcd. for C₇₅H₁₁₈N₂₂O₂₉S [M+H]⁺ m/z=1823.95, [M+2H]²⁺ m/z=912.97, found 913.11 (product after purification).

OPA-Cyclization

Then cyclic peptide cyclo-(CSSLDEPGRGGFSSESKV) (SEQ ID NO:12) (0.5 mg, 1 equiv.) was followed by the OPA-cyclization conditions as described above, the desired bicyclic peptide was obtained by LC-MS.

UV trace from LC-MS analysis of the OPA-cyclization reaction product at 15 min, bicyclo-(CSSLDEPGRGGFSSESKV) (SEQ ID NO:12) shows OPA-cyclization reaction proceeds cleanly to afford bicyclic products under physiological conditions (gradient: 5%-95% ACN/H₂O with 0.1% TFA over 10 min at a flow rate of 0.4 mL/min). ESI-MS calcd. for C₈₃H₁₂₀N₂₂O₂₉S [M+H]⁺ m/z=1923.05, [M+2H]²⁺ m/z=961.53, found 961.87.

Bicyclo-(CSQGTFTSDYSKYLDSRRAQ) (SEQ ID NO:13)

Scheme of bicyclo-(CSQGTFTSDYSKYLDSRRAQ) (7b) (SEQ ID NO:13) formation via NCL and OPA cyclization:

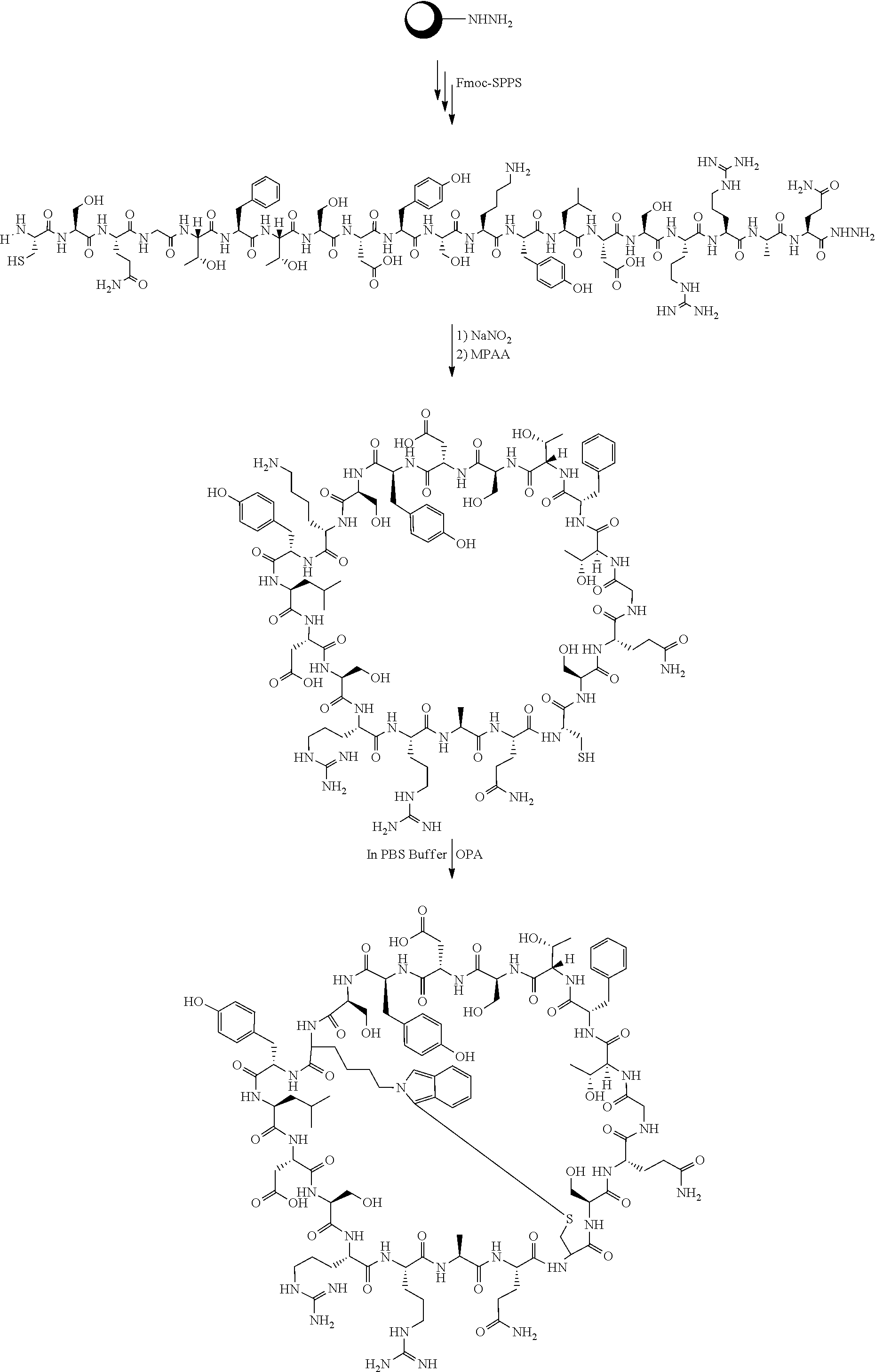

Peptide Synthesis

Peptide NH₂—CSQGTFTSDYSKYLDSRRAQ-CONHNH₂ (SEQ ID NO:35) was synthesized by the general SPPS procedure on the hydrazine 2-chlorotrityl chloride resin. Preparative HPLC purification (10%-60% ACN/H₂O with 0.1% TFA over 45 min), then concentrated under vacuum and lyophilization to NH₂-CSQGTFTSDYSKYLDSRRAQ-CONHNH₂ (SEQ ID NO:35) as a white powder.

NCL-Cyclization

The peptide NH₂—CSQGTFTSDYSKYLDSRRAQ-CONHNH₂ (SEQ ID NO:35) (9.3 mg, 3.99 μmol) was dissolved in pH=3.0 NCL buffer (containing 6.0 mol/L Gn.HCl and 0.2 mol/L NaH₂PO₄, 20 mL) completely, then the reaction mixture was cooled down to −15° C. Then slowly add 0.2 M NaNO₂ solution (70 μL) into the reaction mixture. After the reaction was stirred at −15° C. for 15 min, the 0.2 M MPAA solution (in NCL buffer, pH=7, 0.7 mL) was added into the reaction mixture. The pH value of the ligation mixture was carefully adjusted to 6.8 to 7.0 with 1.0 M NaOH solution, the reaction was allowed to warm up and stirred at room temperature for 5 h to 7 h. After the reaction was finished, 20.0 equiv. of TCEP.HCl (0.1 M) in pH=7.0 phosphate buffer was added to reduce the reaction. Then the reaction was monitored by UPLC. The reaction mixture would be diluted by ACN/H₂O before HPLC purification. Preparative HPLC purification (15%-60% ACN/H₂O with 0.1% TFA over 45 min) followed by concentration under vacuum and lyophilization afforded cyclo-(CSQGTFTSDYSKYLDSRRAQ) (SEQ ID NO:13) (6.6 mg, 2.87 μmol, 71.7%) as a white powder.

UV trace from LC-MS analysis of the crude NCL cyclic reaction mixture, cyclo-(CSQGTFTSDYSKYLDSRRAQ) (SEQ ID NO:13), shows a clean NCL cyclization reaction (gradient: 5%-95% ACN/H₂O with 0.1% TFA over 10 min at a flow rate of 0.4 mL/min). ESI-MS calcd. for C₉₇H₁₄₇N₂₉O₃₄S [M+H]⁺ m/z=2296.47, [M+2H]²⁺ m/z=1148.23, [M+3H]³⁺ m/z=766.49, found 766.48, 1148.80 (crude product).

UV trace from LC-MS analysis of cyclo-(CSQGTFTSDYSKYLDSRRAQ) (SEQ ID NO:13) after purification shows pure NCL-cyclized product (gradient: 5%-95% ACN/H₂O with 0.1% TFA over 5 min at a flow rate of 0.4 mL/min). ESI-MS calcd. for C₉₇H₁₄₇N₂₉O₃₄S [M+H]⁺ m/z=2296.47, [M+2H]²⁺ m/z=1148.23, [M+3H]³⁺ m/z=766.49, found 766.48, 1148.97 (product after purification).

OPA-Cyclization

Then cyclic peptide cyclo-(CSQGTFTSDYSKYLDSRRAQ) (SEQ ID NO:13) (0.65 mg, 1 equiv.) was followed by the OPA-cyclization conditions as described above, the desired bicyclic peptide was obtained by LC-MS.

UV trace from LC-MS analysis of the OPA-cyclization reaction product at 15 min, bicyclo-(CSQGTFTSDYSKYLDSRRAQ) (SEQ ID NO:13), shows OPA-cyclization reaction proceeds cleanly to afford bicyclic products under physiological conditions (gradient: 5%-95% ACN/H₂O with 0.1% TFA over 10 min at a flow rate of 0.4 mL/min). ESI-MS calcd. for C₁₀₅H₁₄₉N₂₉O₃₄S [M+H]⁺ m/z=2394.57, [M+2H]²⁺ m/z=1197.28, [M+3H]³⁺ m/z=798.19, found 798.99, 1197.56.

Bicyclo-(CNSTKNLTFAMRSSGDYGEV) (SEQ ID NO:14)

Scheme of bicyclo-(CNSTKNLTFAMRSSGDYGEV) (7c) (SEQ ID NO:14) formation via NCL and OPA cyclization:

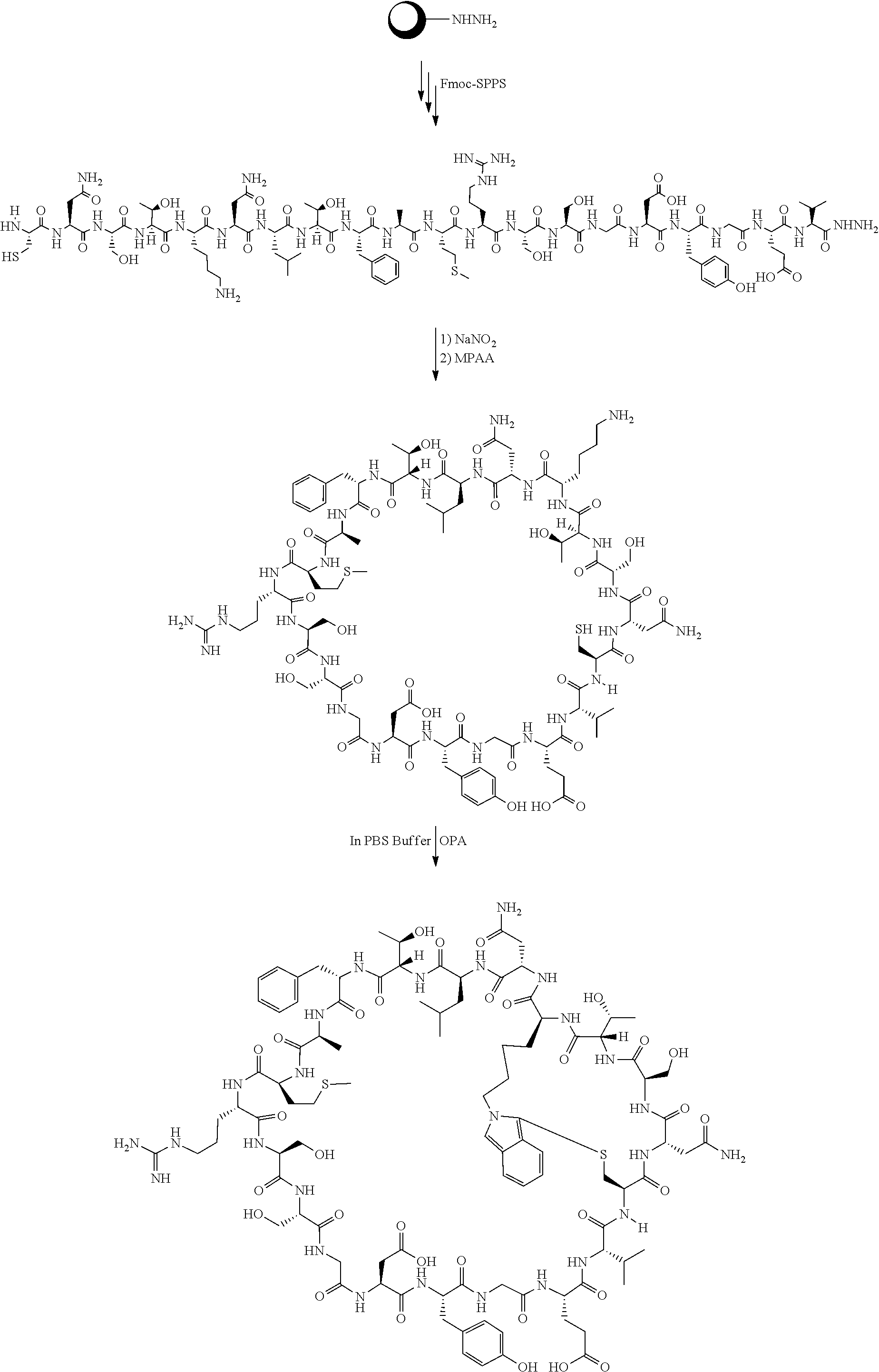

Peptide Synthesis

Peptide NH₂—CNSTKNLTFAMRSSGDYGEV-CONHNH₂ (SEQ ID NO:36) was synthesized by the general SPPS procedure on the hydrazine 2-chlorotrityl chloride resin. Preparative HPLC purification (10%-60% ACN/H₂O with 0.1% TFA over 45 min), then concentrated under vacuum and lyophilization to NH₂-CNSTKNLTFAMRSSGDYGEV-CONHNH₂ (SEQ ID NO:36) as a white powder.

NCL-Cyclization

The peptide NH₂—CNSTKNLTFAMRSSGDYGEV-CONHNH₂ (SEQ ID NO:36) (16.4 mg, 7.44 μmol) was dissolved in pH=3.0 NCL buffer (containing 6.0 mol/L Gn.HCl and 0.2 mol/L NaH₂PO₄, 20 mL) completely, then the reaction mixture was cooled down to −15° C. Then slowly add 0.2 M NaNO₂ solution (80 μL) into the reaction mixture. After the reaction was stirred at −15° C. for 15 min, the 0.2 M MPAA solution (in NCL buffer, pH=7, 0.8 mL) was added into the reaction mixture. The pH value of the ligation mixture was carefully adjusted to 6.8 to 7.0 with 1.0 M NaOH solution, the reaction was allowed to warm up and stirred at room temperature for 6 h. After the reaction was finished, 20.0 equiv. of TCEP.HCl (0.1 M) in pH=7.0 phosphate buffer was added to reduce the reaction. Then the reaction was monitored by UPLC. The reaction mixture would be diluted by ACN/H₂O before HPLC purification. Preparative HPLC purification (20%-60% ACN/H₂O with 0.1% TFA over 45 min) followed by concentration under vacuum and lyophilization afforded cyclo-(CNSTKNLTFAMRSSGDYGEV) (SEQ ID NO:14) (4.9 mg, 2.27 μmol, 31%) as a white powder.

UV trace from LC-MS analysis of the crude NCL cyclic reaction mixture, cyclo-(CNSTKNLTFAMRSSGDYGEV) (SEQ ID NO:14), shows a clean NCL cyclization reaction (gradient: 5%-95% ACN/H₂O with 0.1% TFA over 10 min at a flow rate of 0.4 mL/min). ESI-MS calcd. for C₉₀H₁₄₀N₂₆O₃₂S₂[M+H]⁺ m/z=2163.8, [M+2H]²⁺ m/z=1082.9, [M+3H]³⁺ m/z=721.5, found 1082.0, 721.96 (crude product).

UV trace from LC-MS analysis of cyclo-(CNSTKNLTFAMRSSGDYGEV) (SEQ ID NO:14) after purification shows pure NCL-cyclized product (gradient: 5%-95% ACN/H₂O with 0.1% TFA over 5 min at a flow rate of 0.4 mL/min). ESI-MS calcd. for C₉₀H₁₄₀N₂₆O₃₂S₂[M+H]⁺ m/z=2163.8, [M+2H]²⁺ m/z=1082.9, [M+3H]³⁺ m/z=721.5, found 1081.92, 721.96 (product after purification).

OPA-Cyclization

Then cyclic peptide cyclo-(CNSTKNLTFAMRSSGDYGEV) (SEQ ID NO:14) (2 mg, 1 equiv.) was followed by the OPA-cyclization conditions as described in the general procedure, the desired bicyclic peptide was obtained by LC-MS.

UV trace from LC-MS analysis of the OPA-cyclization reaction product at 15 min, bicyclo-(CNSTKNLTFAMRSSGDYGEV) (SEQ ID NO:14), shows OPA-cyclization reaction proceeds cleanly to afford bicyclic products under physiological conditions (gradient: 5%-95% ACN/H₂O with 0.1% TFA over 10 min at a flow rate of 0.4 mL/min). ESI-MS calcd. for C₉₈H₁₄₂N₂₆O₃₂S₂[M+H]⁺ m/z=2261.48, [M+2H]²⁺ m/z=1131.74, found 1131.27.

Results

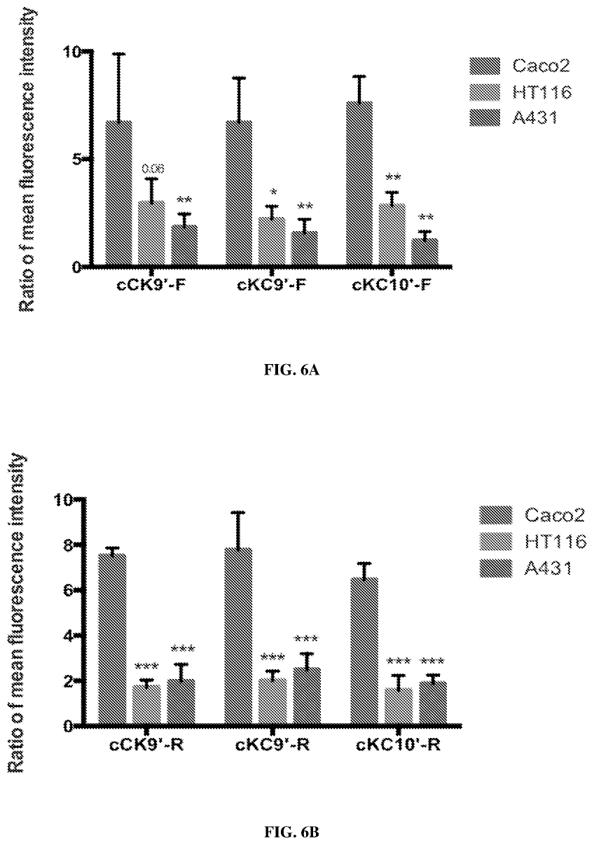

Peptide bicyclization strategy via native chemical ligation (NCL) followed by the OPA-cyclization was demonstrated (FIGS. 6A-6C). Three peptide hydrazides with N-terminal cysteine were readily prepared by Fmoc-solid phase peptide synthesis (Fmoc-SPPS). The peptide thioesters obtained from the peptide hydrazides smoothly cyclized via intramolecular native chemical ligation (NCL). Subsequently, OPA-cyclization gave rise to the bicyclic products cleanly with >90% conversion analyzed by LCMS (See Table 3).

TABLE 3
Bicyclization with different peptide sequences

| No. | NCL-product | Cal. MS | Obtained MS | Conversion |
|---|---|---|---|---|
| 7a | bicyclo- | [M + 2H]²⁺ = 960.97 | 961.87 | >98% |
| | (CSSLDEPGRGGFSSESKV) | | | |
| | (SEQ ID NO: 12) | | | |
| 7b | bicyclo- | [M + 2H]²⁺ = 1196.5; | 1197.56; 798.99 | 94% |
| | (CSQGTFTSDYSKYLDSRRAQ) | [M + 3H]³⁺ = 797.96 | | |
| | (SEQ ID NO: 13) | | | |
| 7c | bicyclo- | [M + 2H]²⁺ = 1130.0 | 1131.27 | 90% |
| | (CNSTKNLTFAMRSSGDYGEV) | | | |
| | (SEQ ID NO: 14) | | | |

### Example 5. OPA-Cyclization Serves as a Useful Handle for Further Derivatization: Post-Modification with Dimethyl Acetylenedicarboxylate (DMAC)

Material and Methods

Scheme of the DMAC mediated post-modification of OPA-cyclization:

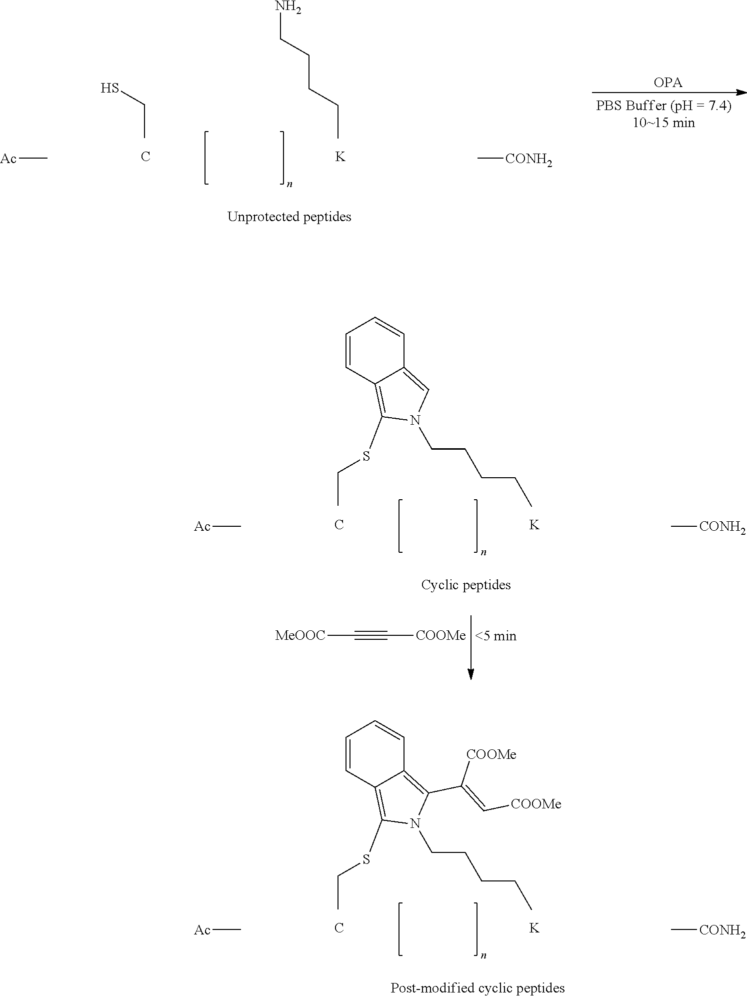

In all OPA-cyclization followed DMAC post-modification experiments, model peptide or protein (1 equiv.) was dissolved into PBS Buffer (pH=7.4) with final concentration of 0.5 mM˜1 mM in 15 mL Eppendorf tube. The ortho-phthaldialdehyde (OPA) (1.3 equiv.) in DMSO was added into the reaction mixture, then stirred at room temperature for 10˜15 min and monitored by analytical RP-UPLC. The dimethyl acetylenedicarboxylate (DMAC, 1.1 equiv.) in DMSO was added into the reaction mixture, then stirred at room temperature for 2˜5 min and monitored by analytical RP-UPLC until reaction was completed. The two steps reaction can be finished within 20 min. The reaction was diluted by H₂O/ACN and then purified by preparative HPLC, concentrated under vacuum and lyophilization to afford desired product.

Cyclo-(Ac-KAAAACH-CONH₂)-DMAC (9a) (SEQ ID NO:15)

Step 1: Ac-KAAAACH-CONH₂ (SEQ ID NO:15) (3.8 mg, 5.3 μmol, 1.0 equiv.) was dissolved in 10 mL PBS Buffer (pH=7.4) with final concentration of 0.53 mM. The Phthaldialdehyde (OPA) (0.923 mg, 6.8 mmol, 1.3 equiv.) in 25 μL DMSO was added into the reaction mixture, then stirred at room temperature for 15 min and monitored by analytical RP-UPLC.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₃₇H₅₁N₁₁O₈S [M+H]⁺ m/z=810.94, [M+2H]²⁺ m/z=405.47, found 810.61, 405.95.

Step 2: After first step OPA-cyclization reaction was complete, the dimethyl acetylenedicarboxylate (0.82 mg, 5.8 mmol, 1.1 equiv.) in 20 μL DMSO was added into the same reaction mixture, then stirred at room temperature for 5 min and monitored the reaction by RP-UPLC until the reaction was completed. The reaction mixture was purified by preparative RP-HPLC (10%-45% ACN/H₂O with 0.1% TFA over 45 min), then concentrated under vacuum and lyophilization to cyclo-(Ac-KAAAACH-CONH₂)-DMAC (SEQ ID NO:15) (1.7 mg, 34% yield) as a red powder.

UV trace from LC-MS analysis of step 2 DMAC post-modification reaction at 5 min shows OPA-cyclization followed DMAC post-modification proceed cleanly in a one-pot manner (gradient: 5%-95% ACN/H₂O with 0.1% TFA over 10 min at a flow rate of 0.4 m/min). ESI-MS calcd. for C₄₃H₅₇N₁₁O₁₂S [M+H]⁺ m/z=952.05, [M+2H]²⁺ m/z=476.02, found 952.57, 476.90.

UV trace from LC-MS analysis of DMAC-modified cyclic peptide c cyclo-(Ac-KAAAACH-CONH₂)-DMAC (SEQ ID NO:15) after purification shows pure one-pot OPA-cyclization followed DMAC post-modification product. Gradient: 5%-95% ACN/H₂O with 0.1% TFA over 5 min at a flow rate of 0.4 mL/min). ESI-MS calcd. for C₄₃H₅₇N₁₁O₁₂S [M+H]⁺ m/z=952.05, [M+2H]²⁺ m/z=476.02, found 952.73, 476.02.

Cyclo-(Ac-KAAACH-CONH₂)-DMAC (9b) (SEQ ID NO:16) Ac-KAAACH-CONH₂ (SEQ ID NO:16) (4 mg, 6.25 mmol) was subjected to the OPA-cyclization (Step 1) and post-modification (Step 2) conditions as described above and monitored by UPLC until the reaction was finished. Preparative HPLC purification (5%-50% ACN/H₂O with 0.1% TFA over 45 min), then concentrated under vacuum and lyophilization to cyclo-(Ac-KAAACH-CONH₂)-DMAC (SEQ ID NO:16) (2.6 mg, 47.27% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₃₄H₄₆N₁₀O₇S [M+H]⁺ m/z=738.87, [M+2H]²⁺ m/z=369.40, found 739.64, 370.43.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₄₀H₅₂N₁₀O₁₁S [M+H]⁺ m/z=880.98, [M+2H]²⁺ m/z=440.49, found 881.61, 441.55.

UV trace from LC-MS analysis of DMAC-modified cyclic peptide cyclo-(Ac-KAAACH-CONH₂)-DMA (SEQ ID NO:16) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₄₀H₅₂N₁₀O₁₁S [M+H]+nm/z=880.98, [M+2H]²⁺ m/z=440.49, found 881.61, 441.56.

Cyclo-(Ac-KAAAAACH-CONH₂)-DMAC (9c) (SEQ ID NO:17) Ac-KAAAAACH-CONH₂ (SEQ ID NO:17) (6 mg, 7.67 μmol) was subjected to the OPA-cyclization (Step 1) and post-modification (Step 2) conditions as described above and monitored by UPLC until the reaction was finished. Preparative HPLC purification (5%-50% ACN/H₂O with 0.1% TFA over 45 min), then concentrated under vacuum and lyophilization to cyclo-(Ac-KAAAAACH-CONH₂)-DMAC (SEQ ID NO:17) (4.0 mg, 52.2% yield) as a red powder.

UV trace from LC-MS analysis of step 1 OPA-cyclization reaction at 10 min shows clean OPA-cyclization reaction product without any starting material remaining. Gradient: 10%-95% ACN/H₂O with 0.1% TFA over 10 min at a flow rate of 0.4 mL/min. ESI-MS calcd. for C₄₀H₅₆N₁₂O₉S [M+H]⁺ m/z=881.02, [M+2H]²⁺ m/z=440.51, found 881.71, 441.42.

UV trace from LC-MS analysis of step 2 DMAC post-modification reaction at 5 min shows OPA-cyclization followed DMAC post-modification proceed cleanly in a one-pot manner. Gradient: 10%-95% ACN/H₂O with 0.1% TFA over 10 min at a flow rate of 0.4 mL/min. ESI-MS calcd. for C₄₆H₆₂N₁₂O₁₃S [M+H]⁺ m/z=1023.13, [M+2H]²⁺ m/z=511.56, found 1023.50, 512.54.

UV trace from LC-MS analysis of DMAC-modified cyclic peptide cyclo-(Ac-KAAAAACH-CONH₂)-DMAC (SEQ ID NO:17) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₄₆H₆₂N₁₂O₁₃S [M+H]⁺ m/z=1023.13, [M+2H]²⁺ m/z=511.56, found 1023.67, 512.58.

Bicyclo-(Dap-Ala-Hyp-Cys-(D)thr-Ala-Glu)-DMAC (9d) (SEQ ID NO:18) The linear phalloidin analog was synthesis by following the general Fmoc-Solid phase peptide synthesis procedure. After synthesis was finished, the resin was treated by a mixture of 5 mL of DCM/AcOH/TFE (v/v/v=8:1:1) for 2 hours to get the protected crude linear phalloidin analog as a white powder.

Then the crude peptide (9.6 mg, 6.09 μmol) was re-dissolved into 900 mL of anhydrous DCM. A mixture of HOAT (4.9 mg, 0.036 mmol), OxymaPure (5.19 mg, 0.0365 mmol) and DIEA (12.7 μL) in 15 mL anhydrous DCM was dropwise added into the reaction mixture with ice bath for 10 min. Then HATU (28.1 mg, 0.074 mmol) was gradually added, the reaction was stirred and allowed to warm up to room temperature. After the reaction was stirred for overnight, DCM was removed under vacuum. The mixture of 10 mL TFA/Phenol/H₂O (v/v/v=95:2.5:2.5) was added into the residue for 2 h. Then removed the TFA solution by a condensed air stream and the residue peptide was washed by cold Et₂O (35 mL×3). Preparative HPLC purification (5%-60% ACN/H₂O with 0.1% TFA over 45 min), then concentrated under vacuum and lyophilization to Phalloidin analog (2.5 mg) as a white powder.

Phalloidin analog (4.5 mg, 6.67 μmol) was subjected to the cyclization (Step 1) and post-modification (Step 2) conditions as described above and monitored by UPLC until the reaction was finished. Preparative HPLC purification (15%-50% ACN/H₂O with 0.1% TFA over 45 min), then concentrated under vacuum and lyophilization to bicyclo-(Dap-A-Hyp-C-t-A-E)-DMAC (SEQ ID NO:18) (3.9 mg, 4.26 μmol, 65%) as a red powder.

UV trace and corresponding MS from LC-MS analysis of step 1 OPA-cyclization reaction at 10 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₃₄H₄₄N₈O₁₁S [M+H]⁺ m/z=772.83, found 773.53.

UV trace and corresponding MS from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 mL/min. ESI-MS calcd. for C₄₀H₅₀N₈O₁₅S [M+H]⁺ m/z=914.94, found 915.57.

UV trace and corresponding MS from LC-MS analysis of DMAC-modified cyclic peptide bicyclo-(Dap-A-Hyp-C-t-A-E)-DMAC (SEQ ID NO:18) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₄₀H₅₀N₈O₁₅S [M+H]⁺ m/z=914.94, found 915.65.

Cyclo-(Ac-ENPECILDKHVQRVM-CONH₂)-DMAC (9e) (SEQ ID NO:10)

Ac-ENPECILDKHVQRVM-CONH₂ (SEQ ID NO:10) (6 mg, 3.23 μmol) was subjected to the cyclization (Step 1) and post-modification (Step 2) conditions as described above and monitored by UPLC until the reaction was finished. Preparative HPLC purification (10%-65% ACN/H₂O with 0.1% TFA over 45 min), then concentrated under vacuum and lyophilization to cyclo-(Ac-ENPECILDKHVQRVM-CONH₂)-DMAC (SEQ ID NO:10) (2.2 mg, 35% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₄₀H₅₀N₈O₁₅S [M+H]⁺ m/z=1950.26, [M+2H]²⁺ m/z=975.13, [M+3H]³⁺ m/z=651.00, found 976.18, 651.43.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₄₀H₅₀N₈O₁₅S [M+H]⁺ m/z=2092.37, [M+2H]²⁺ m/z=1047.18, [M+3H]³ m/z=698.45, found 1047.38, 698.41.

UV trace from LC-MS analysis of DMAC-modified cyclic peptide cyclo-(Ac-ENPECILDKHVQRVM-CONH₂)-DMAC (SEQ ID NO:10) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₄₀H₅₀N₈O₁₅S [M+H]⁺ m/z=2092.37, [M+2H]²⁺ m/z=1047.18, [M+3H]³⁺ m/z=698.45, found 1047.29, 698.33.

Cyclo-(Ac-CDWLPK-CONH₂)-DMAC (9f) (SEQ ID NO:19)

Ac-CDWLPK-CONH₂ (SEQ ID NO:19) (5.6 mg, 6.9 μmol) was subjected to the cyclization (Step 1) and post-modification (Step 2) conditions as described above and monitored by UPLC until the reaction was finished. Preparative HPLC purification (5%-70% ACN/H₂O with 0.1% TFA over 45 min), then concentrated under vacuum and lyophilization to cyclo-(Ac-CDWLPK-CONH₂)-DMAC (SEQ ID NO:19) (2.3 mg, 2.22 μmol, 31.9% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₄₀H₅₀N₈O₁₅S [M+H]⁺ m/z=900.06, found 900.76.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₅₁H₆₃N₉O₁₃S [M+H]⁺ m/z=1042.17, [M+2H]²⁺ m/z=522.08, [M+3H]³⁺ m/z=698.45, found 1042.64, 522.19.

UV trace and corresponding MS from LC-MS analysis of DMAC-modified cyclic peptide cyclo-(Ac-CDWLPK-CONH₂)-DMAC (SEQ ID NO:19) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₅₁H₆₃N₉O₁₃S [M+H]⁺ m/z=1042.17, [M+2H]²⁺ m/z=522.08, [M+3H]³⁺ m/z=698.45, found 1042.72, 521.98.

Cyclo-(Ac-ACFALPKG-CONH₂)-DMAC (9g) (SEQ ID NO:20) Ac-ACFALPKG-CONH₂ (SEQ ID NO:20) (5.7 mg, 7.1 μmol) was subjected to the cyclization (Step 1) and post-modification (Step 2) conditions as described above and monitored by UPLC until the reaction was finished. Preparative HPLC purification (5%-55% ACN/H₂O with 0.1% TFA over 45 min), then concentrated under vacuum and lyophilization to cyclo-(Ac-ACFALPKG-CONH₂)-DMAC (SEQ ID NO:20) (3.7 mg, 3.41 μmol, 48% yield) as a red powder.

UV trace from LC-MS analysis of step 1 OPA-cyclization reaction at 10 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₄₇H₆₄N₁₀O₉S [M+H]⁺ m/z=945.15, [M+2H]²⁺ m/z=473.07, found 945.70, 473.64.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₅₃H₇₀N₁₀O₁₃S [M+H]⁺ m/z=1087.26, [M+2H]²⁺ m/z=543.63, found 1087.84, 544.58.

UV trace from LC-MS analysis of DMAC-modified cyclic peptide cyclo-(Ac-ACFALPKG-CONH₂)-DMAC (SEQ ID NO:20) after purification shows pure one-pot OPA-cyclization followed DMAC post-modification product (See FIG. G). Gradient: 5%-95% ACN/H₂O with 0.1% TFA over 5 min at a flow rate of 0.4 mL/min. ESI-MS calcd. for C₅₃H₇₀N₁₀O₁₃S [M+H]⁺ m/z=1087.26, [M+2H]²⁺ m/z=543.63, found 1087.74, 544.58.

Cyclo-(Ac-KGEAFQC-CONH₂)-DMAC (9h) (SEQ ID NO:21)

Ac-KGEAFQC-CONH₂ (SEQ ID NO:21) (5.6 mg, 6.8 μmol) was subjected to the cyclization (Step 1) and post-modification (Step 2) conditions as described above and monitored by UPLC until the reaction was finished. Preparative HPLC purification (10%-50% ACN/H₂O with 0.1% TFA over 45 min), then concentrated under vacuum and lyophilization to cyclo-(Ac-KGEAFQC-CONH₂)-DMAC (SEQ ID NO:21) (2.6 mg, 2.45 μmol, 36.6% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₄₃H₅₆N₁₀O₁₁S [M+H]⁺ m/z=921.04, [M+2H]²⁺ m/z=461.52, found 921.84, 461.80.

UV trace from LC-MS analysis of step 2 DMAC post-modification reaction at 4 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₄₉H₆₂N₁₀O₁₅S [M+H]⁺ m/z=1063.15, [M+2H]²⁺ m/z=532.57, found 1063.88, 532.68.

UV trace from LC-MS analysis of DMAC-modified cyclic peptide cyclo-(Ac-KGEAFQC-CONH₂)-DMAC (SEQ ID NO:21) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₄₉H₆₂N₁₀O₁₅S [M+H]⁺ m/z=1063.15, [M+2H]²⁺ m/z=532.57, found 1063.80, 532.56.

Cyclo-(Ac-GAQCAFLK-CONH₂)-DMAC (9i) (SEQ ID NO:22) Ac-GAQCAFLK-CONH₂ (SEQ ID NO:22) (6.0 mg, 6.7 μmol) was subjected to the cyclization (Step 1) and post-modification (Step 2) conditions as described above and monitored by UPLC until the reaction was finished. Preparative HPLC purification (5%-50% ACN/H₂O with 0.1% TFA over 45 min), then concentrated under vacuum and lyophilization to cyclo-(Ac-GAQCAFLK-CONH₂)-DMAC (SEQ ID NO:22) (2.8 mg, 2.5 μmol, 38% yield) as a red powder.

UV trace from LC-MS analysis of step 1 OPA-cyclization reaction at 10 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₄₇H₆₅N₁₁O₁₀S [M+H]⁺ m/z=976.16, found 976.86.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₅₃H₇₁N₁₁O₁₄S [M+H]⁺ m/z=1118.27, [M+2H]²⁺ m/z=560.13, found 1118.74, 560.11.

UV trace and corresponding MS from LC-MS analysis of DMAC-modified cyclic peptide cyclo-(Ac-GAQCAFLK-CONH₂)-DMAC (SEQ ID NO:22) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₅₃H₇₁N₁₁O₁₄S [M+H]⁺ m/z=1118.27, [M+2H]²⁺ m/z=560.13, found 1118.66, 560.08.

Cyclo-(Ac-AKVTMTCSAS-CONH₂)-DMAC (9j) (SEQ ID NO:23)

Ac-AKVTMTCSAS-CONH₂ (SEQ ID NO:23) (4.15 mg, 4.0 μmol) was subjected to the cyclization (Step 1) and post-modification (Step 2) conditions as described above and monitored by UPLC until the reaction was finished. Preparative HPLC purification (20%-45% ACN/H₂O with 0.1% TFA over 45 min), then concentrated under vacuum and lyophilization to cyclo-(Ac-AKVTMTCSAS-CONH₂)-DMAC (SEQ ID NO:23) (1.6 mg, 1.25 μmol, 32% yield) as a red powder.

UV trace from LC-MS analysis of step 1 OPA-cyclization reaction at 20 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₄₉H₇₆N₁₂O₁₅S₂[M+H]⁺ m/z=1137.33, [M+2H]²⁺ m/z=569.66, found 976.86, 569.47.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₅₅H₈₂N₁₂O₁₉S₂[M+H]⁺ m/z=1279.44, [M+2H]²⁺ m/z=640.72, found 1279.68, 640.67.

UV trace from LC-MS analysis of DMAC-modified cyclic peptide cyclo-(Ac-AKVTMTCSAS-CONH₂)-DMAC (SEQ ID NO:23) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₅₅H₈₂N₁₂O₁₉S₂[M+H]⁺ m/z=1279.44, [M+2H]²⁺ m/z=640.72, found 1279.52, 640.50.

Cyclo-(Ac—NYRWRCKN-CONH₂)-DMAC (9k) (SEQ ID NO:24) Ac-AKVTMTCSAS-CONH₂ (SEQ ID NO:24) (4.45 mg, 3.8 μmol) was subjected to the cyclization (Step 1) and post-modification (Step 2) conditions as described above and monitored by UPLC until the reaction was finished. Preparative HPLC purification (5%-50% ACN/H₂O with 0.1% TFA over 45 min), then concentrated under vacuum and lyophilization to cyclo-(Ac—NYRWRCKN-CONH₂)-DMAC (SEQ ID NO:24) (1.7 mg, 1.2 μmol, 32% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₅₉H₇₉N₁₉O₁₂S [M+H]⁺ m/z=1278.46, [M+2H]²⁺ m/z=640.23, found 640.17.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₆₅H₈₅N₁₉O₁₆S [M+H]⁺ m/z=1420.57, [M+2H]²⁺ m/z=711.28, found 711.53.

UV trace from LC-MS analysis of DMAC-modified cyclic peptide cyclo-(Ac—NYRWRCKN-CONH₂)-DMAC (SEQ ID NO:24) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₆₅H₈₅N₁₉O₁₆S [M+H]⁺ m/z=1420.57, [M+2H]²⁺ m/z=711.28, found 711.20.

Bicyclo-(CSSLDEPGRGGFSSESKV)-DMAC (9l) (SEQ ID NO:12)

Cyclo-(CSSLDEPGRGGFSSESKV) (SEQ ID NO:12) (1.5 mg) was subjected to the cyclization (Step 1) and post-modification (Step 2) conditions as described above and monitored by UPLC until the reaction was finished. Preparative HPLC purification (10%-55% ACN/H₂O with 0.1% TFA over 45 min), then concentrated under vacuum and lyophilization to bicyclo-(CSSLDEPGRGGFSSESKV)-DMAC (SEQ ID NO:12) (0.7 mg, 43% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₈₃H₁₂₀N₂₂O₂₉S [M+H]⁺ m/z=1923.05, [M+2H]²⁺ m/z=961.53, found 961.87.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₈₉H₁₂₆N₂₂O₃₃S [M+H]⁺ m/z=2065.16, [M+2H]²⁺ m/z=1032.58, found 1032.90.

UV trace from LC-MS analysis of DMAC-modified bicyclic peptide, bicyclo-(CSSLDEPGRGGFSSESKV)-DMAC (SEQ ID NO:12), after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₈₉H₁₂₆N₂₂O₃₃S [M+H]⁺ m/z=2065.16, [M+2H]²⁺ m/z=1032.58, found 1033.15.

Bicyclo-(CSQGTFTSDYSKYLDSRRAQ)-DMAC (9m) (SEQ ID NO:13)

Cyclo-(CSQGTFTSDYSKYLDSRRAQ) (SEQ ID NO:13) (2.6 mg, 1.132 μmol) was subjected to the cyclization (Step 1) and post-modification (Step 2) conditions as described above and monitored by UPLC until the reaction was finished. Preparative HPLC purification (5%-65% ACN/H₂O with 0.1% TFA over 45 min), then concentrated under vacuum and lyophilization to bicyclo-(CSQGTFTSDYSKYLDSRRAQ)-DMAC (SEQ ID NO:13) (1.9 mg, 0.66 μmol, 65% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₁₀₅H₁₄₉N₂₉O₃₄S [M+H]⁺ m/z=2394.57, [M+2H]²⁺ m/z=1197.28, [M+3H]³⁺ m/z=798.19, found 798.99, 1197.56.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₁₁₁H₁₅₅N₂₉O₃₈S [M+H]⁺ m/z=2536.69, [M+2H]²⁺ m/z=1268.35, [M+3H]³⁺ nm/z=846.00, found 1268.85, 846.06.

UV trace from LC-MS analysis of DMAC-modified bicyclic peptide, bicyclo-(CSQGTFTSDYSKYLDSRRAQ)-DMAC (SEQ ID NO:13), after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₁₁₁H₁₅₅N₂₉O₃₈S [M+H]⁺ m/z=2536.69, [M+2H]²⁺ m/z=1268.35, [M+3H]³⁺ m/z=846.00, found 1268.42, 846.48.

Bicyclo-(CNSTKNLTFAMRSSGDYGEV)-DMAC (9n) (SEQ ID NO:14)

Cyclo-(CNSTKNLTFAMRSSGDYGEV) (SEQ ID NO:14) (6.4 mg, 2.91 μmol) was subjected to the cyclization (Step 1) and post-modification (Step 2) conditions as described above and monitored by UPLC until the reaction was finished. Preparative HPLC purification (10%-55% ACN/H₂O with 0.1% TFA over 45 min), then concentrated under vacuum and lyophilization to bicyclo-(CNSTKNLTFAMRSSGDYGEV)-DMAC (SEQ ID NO:14) (4.9 mg, 2.03 μmol, 69% yield) as a red powder.

UV trace from LC-MS analysis of step 1 OPA-cyclization reaction at 15 min shows clean OPA-cyclization reaction product without any starting material remaining. Gradient: 5%-95% ACN/H₂O with 0.1% TFA over 10 min at a flow rate of 0.4 mL/min. ESI-MS calcd. for C₉₈H₁₄₂N₂₆O₃₂S₂[M+H]⁺ m/z=2261.48, [M+2H]²⁺ m/z=1130.74, found 1131.27.

UV trace from LC-MS analysis of step 2 DMAC post-modification reaction at 5 min shows OPA-cyclization followed DMAC post-modification proceed cleanly in a one-pot manner. Gradient: 5%-95% ACN/H₂O with 0.1% TFA over 10 min at a flow rate of 0.4 m/min. ESI-MS calcd. for C₁₀₄H₁₄₈N₂₆O₃₆S₂[M+H]⁺ m/z=2402.59, [M+2H]²⁺ m/z=1202.79, found 1202.22.

UV trace from LC-MS analysis of DMAC-modified bicyclic peptide bicyclo-(CNSTKNLTFAMRSSGDYGEV)-DMAC (SEQ ID NO:14) after purification shows pure one-pot OPA-cyclization followed DMAC post-modification product. Gradient: 5%-95% ACN/H₂O with 0.1% TFA over 5 min at a flow rate of 0.4 mL/min. ESI-MS calcd. for C₁₀₄H₁₄₈N₂₆O₃₆S₂ [M+H]⁺ m/z=2402.59, [M+2H]²⁺ m/z=1202.79, found 1202.14.

Results

The isoindole moiety after OPA-cyclization serves as a useful handle for further derivatization. The obtained cyclic peptides reacted with either dimethyl acetylenedicarboxylate (DMAC) very rapidly with completion within minutes. By modification of the DMAC, the OPA-cyclization guided post-modification is a module-assembled approach for constructing functional peptide architectures. The OPA-cyclization and post-modification can be performed in one-pot, by sequentially adding OPA and DMAC into the reaction mixture. The resultant product was found to be the addition product (See compounds 9a-9n described above) (Simons, et al., The Journal of Organic Chemistry, 46:(23):4739-4744 (1981); White, et al., Advances in Heterocyclic Chemistry, Elsevier: Vol. 10, pp 113-147 (1969); Simons, et al., Journal of the American Chemical Society, 98(22):7098-7099 (1976); Kreher, et al., Tetrahedron Letters, 14(22):1911-1914 (1973)). This post-cyclization modification can further diversify the structural complexity of the cyclic peptides (See Table 4).

TABLE 4
Results of the DMAC mediated post-
modification of OPA-cyclization with
different peptide sequences.

| | | Conversion | | HPLC |
|---|---|---|---|---|
| | | (%)^{[a]} | | Yield |
| No. | Peptides | Step 1 | Step 2 | (%) |
| 9a. | Ac-KAAAACH-CONH₂ | 92 | 74.2 | 34.4 |
| | (SEQ ID NO: 15) | | | |
| 9b. | Ac-KAAACH-CONH₂ | 98 | 72.1 | 54 |
| | (SEQ ID NO: 16) | | | |
| 9c. | Ac-KAAAAACH-CONH₂ | 98 | 80.4 | 53.2 |
| | (SEQ ID NO: 17) | | | |
| 9d. | Phallodin analog | 86.4 | 68 | 66 |
| | (cyclo-(Dap-Ala- | | | |
| | Hyp-Cys-(D)thr- | | | |
| | Ala-Glu)) | | | |
| | (SEQ ID NO: 18) | | | |
| 9e. | Ac-ENPECILDKH | 93.6 | 76 | 35 |
| | VQRVM-CONH₂ | | | |
| | (SEQ ID NO: 10) | | | |
| 9f. | Ac-CDWLPK-CONH₂ | 89.1 | 53 | 32 |
| | (SEQ ID NO: 19) | | | |
| 9g. | Ac-ACFALPKG-CONH₂ | 92.5 | 90.3 | 48.6 |
| | (SEQ ID NO: 20) | | | |
| 9h. | Ac-KGEAFQC-CONH₂ | 90.9 | 80.4 | 36.6 |
| | (SEQ ID NO: 21) | | | |
| 9i. | Ac-GAQCAFLK-CONH₂ | 83.3 | 71.3 | 38 |
| | (SEQ ID NO: 22) | | | |
| 9j. | Ac-AKVTMTCSAS-CONH₂ | 99 | 80.9 | 32.1 |
| | (SEQ ID NO: 23) | | | |
| 9k. | Ac-NYRWRCKN-CONH₂ | 98 | 84.3 | 28.3 |
| | (SEQ ID NO: 24) | | | |
| 9l. | cyclo-(CSSLDEPGR | 86.9 | 89.8 | 43.7 |
| | GGFSSESKV) | | | |
| | (SEQ ID NO: 12) | | | |
| 9m. | cyclo-(CSQGTFTSDY | 71.4 | 74.2 | 65 |
| | SKYLDSRRAQ) | | | |
| | (SEQ ID NO: 13) | | | |
| 9n. | cyclo-(CNSTKNLTF | 89.2 | 78.1 | 32.5 |
| | AMRSSGDYGEV) | | | |
| | (SEQ ID NO: 14) | | | |
| | Average | 90.59 | 76.6 | 42.7 |

^{[a]}conversion percentage was calculated based on LCMS profile.

### Example 6. The OPA-Cyclization-DMAC Products Show Enhanced Stability

Material and Methods

Model peptide α1 (Ac-ENPECILDKHVQRVM-CONH2) (SEQ ID NO:10) was subjected to OPA cyclization reaction. The OPA-cyclized peptide was purified by RP-HPLC. Model peptide al (Ac-ENPECILDKHVQRVM-CONH2) (SEQ ID NO:10) was subjected to OPA-cyclization and one-pot post-modification by DMAC. The desired DMAC-modified cyclic peptide was purified by RP-HPLC. Both peptides were separately dissolved in PBS buffer (pH=7.4) with a final concentration of 0.5 mM, and then two vessels were placed in room temperature. The stability was calculated by peak area based on LC-MS spectrum (*: P value<0.05, **: P value<0.01, ***: P value<0.001).

Results

The resultant moieties of the OPA-cyclization-DMAC products showed much enhanced stability as compared with the isoindoles that were prone to oxidation over time (See FIG. 2) (White, et al., Advances in Heterocyclic Chemistry, Eds. Katritzky and Boulton, Academic Press: Vol. 10, pp 113-147 (1969); Bonnett, et al., Journal of the Chemical Society, Chemical Communications, 7:393-395 (1972); Simons, et al., Anal. Biochem., 90(2):705-25 (1978)).

### Example 7. OPA-Cyclization Serves as a Useful Handle for Further Derivatization: Post-Modification with Various Maleimide Analogs

Materials and Methods

Scheme of fluorophore-maleimide conjugation with the OPA guided cyclization:

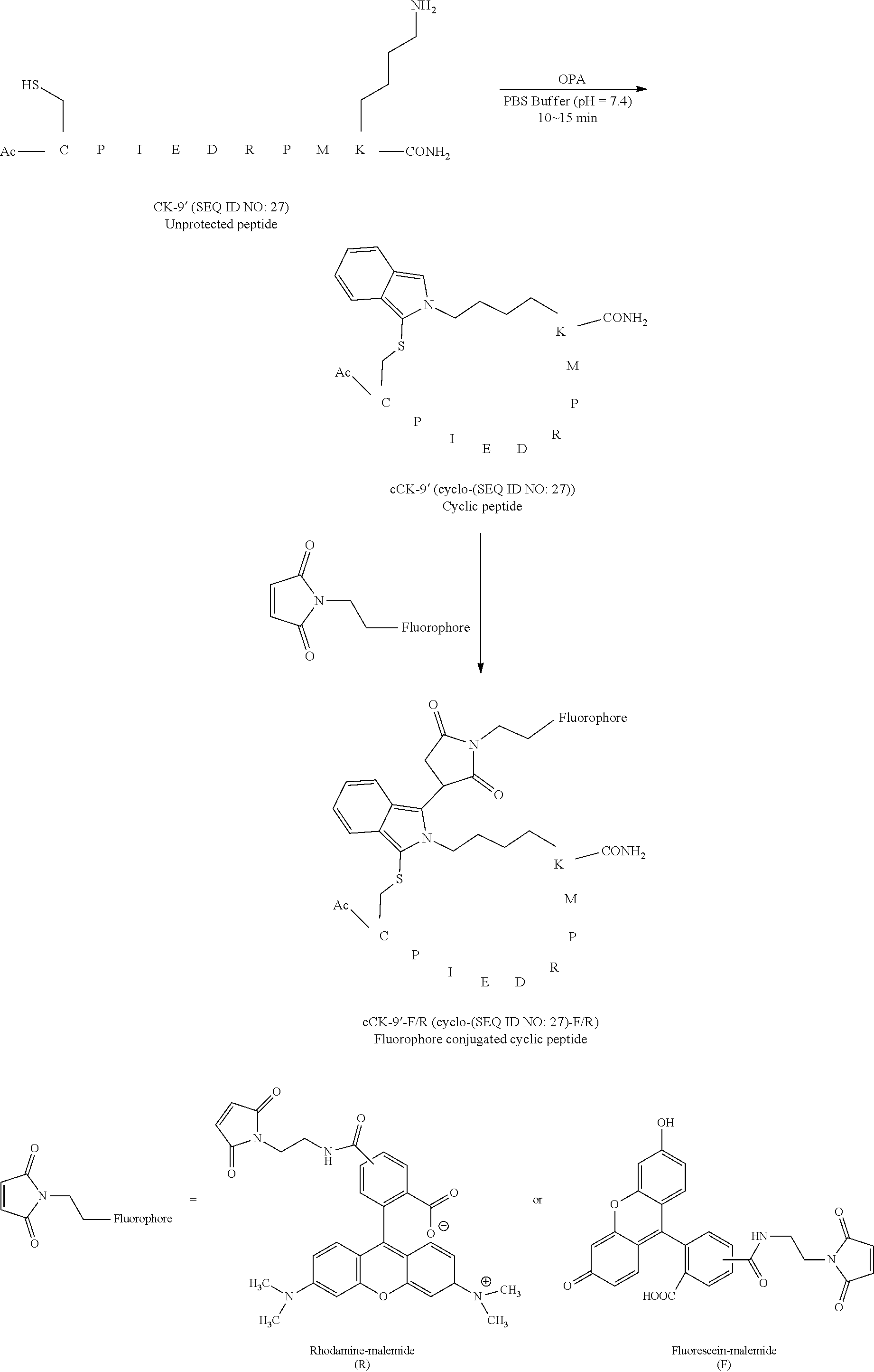

Synthesis of Fluorescein-Maleimide Analogs

The compound, 2-(2-aminoethyl)cyclopent-4-ene-1,3-dione hydrochloride (12a), was prepared following the literature protocol (Richter, et al., Chemistry—A European Journal, 18(52):16708-16715 (2012)).

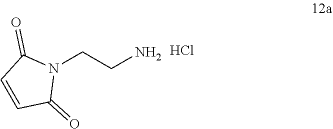

Fluorescein-maleimide (12b)

To a solution of 5(6)-Carboxyfluorescein N-hydroxysuccinimide ester (20 mg, 0.042 mmol) in DMSO/ACN (1 mL/5 mL), DIPEA (7.9 μL, 0.045 mmol), 2-(2-aminoethyl) cyclopent-4-ene-1,3-dione hydrochloride (7.4 mg, 0.042 mmol) were added and the reaction was stirred at room temperature for 2 h. The reaction was monitored by RP-LCMS and diluted by H₂O/ACN (0.1% TFA) for RP-HPLC purification. Preparative HPLC purification (5%-70% ACN/H₂O with 0.1% TFA over 45 min), then concentrated under vacuum and lyophilization to Fluorescein-maleimide (12b) (15.2 mg, 71.5% yield) as a yellow powder.

UV trace from LC-MS analysis of the reaction shows the major peak is desired fluorescein-maleimide. Gradient: 5%-95% ACN/H₂O with 0.1% TFA over 8 min at a flow rate of 0.4 mL/min. ESI-MS calcd. for C₂₇H₁₈N₂O₆[M+H]⁺ m/z=499.44, found 499.29.

UV trace from LC-MS analysis of the purified product shows the pure fluorescein-maleimide (12b) was collected. Gradient: 5%-95% ACN/H₂O with 0.1% TFA over 5 min at a flow rate of 0.4 mL/min. ESI-MS calcd. for C₂₇H₁₈N₂O₆[M+H]⁺ m/z=499.44, found 499.46.

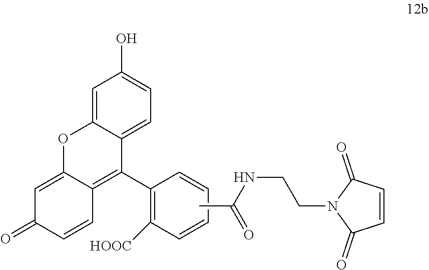

Rhodamine-Maleimide (12c)

To a solution of 5(6)-Carboxytetramethylrhodamine (36 mg, 0.083 mmol) in anhydrous DMF/DCM (2 mL/2 mL), HATU (64.7 mg, 0.083 mmol), DIPEA (79 μL, 0.22 mmol), 2-(2-aminoethyl) cyclopent-4-ene-1,3-dione hydrochloride (10 mg, 0.056 mmol) were added and the reaction was stirred at room temperature for 2 h. The reaction was monitored by RP-LCMS. Preparative HPLC purification (10%-60% ACN/H₂O with 0.1% TFA over 45 min), then concentrated under vacuum and lyophilization to Rhodamine-maleimide (12c) (20.5 mg, 66.3% yield) as a red powder.

UV trace from LC-MS analysis of the reaction shows the two major peaks are the desired product due to two isomers of the 5(6)-Carboxytetramethylrhodamine. Gradient: 5%-95% ACN/H₂O with 0.1% TFA over 8 min at a flow rate of 0.4 mL/min. ESI-MS calcd. for C₃₁H₂₈N₄O₆ [M+H]⁺ m/z=553.58, found 553.47. The two product peaks because of the two isomers of the 5(6)-Carboxytetramethylrhodamine.

UV trace and corresponding MS from LC-MS analysis of the purified product shows pure rhodamine-maleimide (12c) was collected. Gradient: 5%-95% ACN/H₂O with 0.1% TFA over 5 min at a flow rate of 0.4 mL/min. ESI-MS calcd. for C₃₁H₂₈N₄O₆ [M+H]⁺ m/z=553.58, found 553.47. The two product peaks because of the two isomers in the 5(6)-Carboxytetramethylrhodamine.

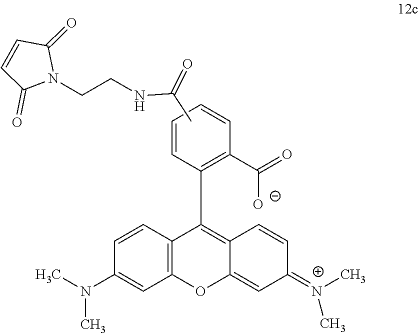

OPA-Cyclization Followed by Post-Modification with Fluorophore-Maleimide Probes

The target peptide (1eq) was dissolved in PBS buffer (pH=7.4) with a final concentration of 0.5 mM. The OPA (ortho-phthalaldehyde) (1.2 equiv.) in DMSO was slowly added and the mixture was stirred at room temperature for 10-15 min. After the cyclization was completed, fluorophore-maleimide probe (1.5 equiv.˜2 equiv.) in DMSO was added to the reaction mixture. The reaction was monitored by LCMS after 5-15 min. Preparative RP-HPLC purification was followed after the reaction was completed.

(SEQ ID NO: 25)
Cyclo-(Ac-KTPSPFDSHC-CONH₂)-
Fluorescein (cKC10′-F)

UV trace from LC-MS analysis of the one-pot reaction shows one-pot OPA-cyclization followed fluorescein post-modification was fully converted. Gradient: 10%-95% ACN/H₂O with 0.1% TFA over 10 min at a flow rate of 0.4 mL/min. ESI-MS calcd. for C₈₅H₉₄N₁₆O₂₄S [M+H]⁺ m/z=1756.8, [M+2H]²⁺ m/z=878.4, [M+3H]³⁺ m/z=586.6, found 586.32, 878.65.

UV trace from LC-MS analysis of purified product shows pure desired fluorescein-modified OPA-cyclized product. Gradient: 5%-95% ACN/H₂O with 0.1% TFA over 5 min at a flow rate of 0.4 mL/min. ESI-MS calcd. for C₈₅H₉₄N₁₆O₂₄S [M+H]⁺ m/z=1756.8, [M+2H]²⁺ m/z=878.4, [M+3H]³⁺ m/z=586.6, found 586.49, 878.16. PGP-76 DNA

(SEQ ID NO: 26)
Cyclo-(Ac-KSDSWHYWC-CONH₂)-
Fluorescein (cKC9′-F)

UV trace and corresponding MS from LC-MS analysis of the one-pot reaction shows one-pot OPA-cyclization followed fluorescein post-modification was fully converted. Gradient: 10%-85% ACN/H₂O with 0.1% TFA over 10 min at a flow rate of 0.4 m/min. ESI-MS calcd. for C₉₃H₉₃N₁₇O₂₃S [M+H]⁺ m/z=1849.9, [M+2H]²⁺ m/z=925.45, [M+3H]³⁺ m/z=617.3, found 617.48, 925.55, 1850.49.

UV trace and corresponding MS from LC-MS analysis of purified product shows pure desired fluorescein-modified OPA-cyclized product. Gradient: 5%-95% ACN/H₂O with 0.1% TFA over 5 min at a flow rate of 0.4 mL/min. ESI-MS calcd. for C₉₃H₉₃N₁₇O₂₃S [M+H]⁺ m/z=1849.9, [M+2H]²⁺ m/z=925.45, [M+3H]³⁺ m/z=617.3, found 617.48, 925.56, 1850.56.

(SEQ ID NO: 27)
Cyclo-(Ac-CPIEDRPMK-CONH₂)-
Fluorescein (cCK9′-F)

UV trace from LC-MS analysis of the one-pot reaction shows one-pot OPA-cyclization followed fluorescein post-modification was fully converted. Gradient: 20%-60% ACN/H₂O with 0.1% TFA over 10 min at a flow rate of 0.4 mL/min. ESI-MS calcd. for C₈₂H₁₀₀N₁₆O₂₂S [M+H]⁺ m/z=1726.9, [M+2H]²⁺ m/z=863.95, [M+3H]³⁺ m/z=576.3, found 576.50, 863.75, 1726.71.

UV trace from LC-MS analysis of purified product shows pure desired fluorescein-modified OPA-cyclized product. Gradient: 5%-95% ACN/H₂O with 0.1% TFA over 5 min at a flow rate of 0.4 mL/min. ESI-MS calcd. for C₈₂H₁₀₀N₁₆O₂₂S [M+H]⁺ m/z=1726.9, [M+2H]²⁺ m/z=863.95, [M+3H]³⁺ nm/z=576.3, found 576.32, 864.18, 1727.55.

(SEQ ID NO: 25)
Cyclo-(Ac-KTPSPFDSHC-CONH₂)-
Rhodamine (cKC10′-R)

UV trace from LC-MS analysis of the one-pot reaction shows one-pot OPA-cyclization followed rhodamine post-modification was fully converted. Gradient: 15%-70% ACN/H₂O with 0.1% TFA over 10 min at a flow rate of 0.4 mL/min. ESI-MS calcd. for C₈₉H₁₀₄N₁₈O₂₂S [M+H]⁺ m/z=1809.97, [M+2H]²⁺ m/z=905.98, [M+3H]³⁺ m/z=604.32, found 604.60, 906.33. The two product peaks because of the two isomers in the 5(6)-Carboxytetramethylrhodamine.

UV trace from LC-MS analysis of purified product shows pure desired rhodamine-modified OPA-cyclized product. Gradient: 5%-95% ACN/H₂O with 0.1% TFA over 5 min at a flow rate of 0.4 mL/min. ESI-MS calcd. for C₈₉H₁₀₄N₁₈O₂₂S [M+H]⁺ m/z=1809.97, [M+2H]²⁺ m/z=905.98, [M+3H]³⁺ m/z=604.32, found 604.52, 906.08.

(SEQ ID NO: 26)
Cyclo-(Ac-KSDSWHYWC-CONH₂)-
Rhodamine (cKC9′-R)

UV trace from LC-MS analysis of the one-pot reaction shows one-pot OPA-cyclization followed rhodamine post-modification was fully converted. Gradient: 15%-45% ACN/H₂O with 0.1% TFA over 10 min at a flow rate of 0.4 mL/min. ESI-MS calcd. for C₈₉H₁₀₄N₁₈O₂₂S [M+H]⁺ m/z=1809.97, [M+2H]²⁺ m/z=952.03, [M+3H]³⁺ m/z=635.02, found 635.59, 952.22. The two product peaks because of the two isomers in the 5(6)-Carboxytetramethylrhodamine.

UV trace from LC-MS analysis of purified product shows pure desired rhodamine-modified OPA-cyclized product. Gradient: 5%-95% ACN/H₂O with 0.1% TFA over 5 min at a flow rate of 0.4 mL/min. ESI-MS calcd. for C₈₉H₁₀₄N₁₈O₂₂S [M+H]⁺ m/z=1809.97, [M+2H]²⁺ m/z=952.03, [M+3H]³⁺ m/z=635.02, found 635.25, 952.55.

(SEQ ID NO: 27)
Cyclo-(Ac-CPIEDRPMK-CONH₂)-
Rhodamine (cCKW-R)

UV trace from LC-MS analysis of the one-pot reaction shows one-pot OPA-cyclization followed rhodamine post-modification was fully converted. Gradient: 10%-60% ACN/H₂O with 0.1% TFA over 10 min at a flow rate of 0.4 mL/min. ESI-MS calcd. for C₈₆H₁₁₀N₁₈O₂₀S₂ [M+H]⁺ m/z=1781.05, [M+2H]²⁺ m/z=891.02, [M+3H]³⁺ m/z=594.35, found 594.27, 891.09. The two product peaks because of the two isomers in the 5(6)-Carboxytetramethylrhodamine.

UV trace from LC-MS analysis of purified product shows pure desired rhodamine-modified OPA-cyclized product. Gradient: 5%-95% ACN/H₂O with 0.1% TFA over 5 min at a flow rate of 0.4 mL/min. ESI-MS calcd. for C₈₆H₁₁₀N₁₈O₂₀S₂ [M+H]⁺ m/z=1781.05, [M+2H]²⁺ m/z=891.02, [M+3H]³⁺ m/z=594.35, found 594.19, 891.01.

Cell Lines

Caco2 cell line was provided by Prof. Jiang Xia (CUHK). A431 and HT116 cell lines were from Prof. Chiming Che (HKU). Caco2, A431 cells were cultured in DMEM supplemented with 10% FBS. HT116 cells were cultured in RPMI 1640 medium supplemented with 10% FBS. All cells were cultured in a humidified incubator at 37° C. with 5% CO₂ supplemented. Cell culture medium and FBS were purchased from Gibco. Hoechst 33342 and DiI were purchased from Beyotime Biotechnology. Flow cytometry tubes were purchased from BD pharmacology.

Fluorescence Confocal Imaging Analysis

Caco2 cells were cultured with the confluence of 60% in the 35 mm glass-bottom dish overnight. 1 mL fresh DMEM with 10% FBS was added to the dish. 1 μL 10 mM cyclic peptide (Ac-CPIEDRPMK-CONH2)-Fluorescein (cCK9′-F) (SEQ ID NO:27) in DMSO was added into the medium for 2 hours at 37° C. After two hours' incubation, the supernatant was discarded and the cells were washed with PBS for three times. The cells were replenished with PBS. Hoechst 33342 (final concentration: 2.5 μg/mL) and DiI (final concentration: 5 μM) were added into PBS for 10 min at room temperature. The supernatant was discarded. The cells were washed with PBS for three times and replenished with 3% polyaldehyde for imaging. All images were captured and processed via Leica Application Suit X at ×63 oil objective (Leica).

Flow Cytometry Analysis

Caco2, HT116, A431 cells were cultured in 10 cm dishes with the confluence of 80-90%, digested, washed with PBS for three times, and re-suspended with PBS. All the cell lines were incubated with 10 μM fluorescein conjugated cyclic peptides (cCK9′-F, cKC9′-F, cKC10′-F) and rhodamine conjugated peptides (cCK9′-R, cKC9′-R, cKC10′-R) separately for 2 hours at 37° C. After two hours' incubation, all samples were washed with PBS for three times and resuspended in 1% polyaldehyde in PBS. All samples were analyzed by FACS Aria (Becton Dickinson, San Jose, Calif.). All FACS data were analyzed using Flowjo 7.6. Each sample was replicated three times. The static data (mean fluorescence intensity) was analyzed by Graphpad Prism 6. (*: P value<0.05, **: P value<0.01, ***: P value<0.001.)

Results

The cyclic peptides obtained from OPA cyclization can react with N-maleimide (NMM) very rapidly with completion within minutes. By modification of N-maleimide, the OPA-cyclization guided post-modification could be a module-assembled approach for constructing functional peptide architectures.

Two modules of N-maleimide-conjugated fluorophores (fluorescein and rhodamine) were synthesized, which could be easily conjugated with OPA-guided cyclic peptides. Cyclic peptide CK-9 cyclo-(CPIEDRPMC) (SEQ ID NO:37) was previously reported to specifically target poorly differentiated colon carcinoma cells (Kelly, et al., Neoplasia, 5(5):437-444 (2003)). To apply OPA cyclization, CK-9 derivative peptide CK-9′ (CPIEDRPMK; SEQ ID NO:27) was first synthesized, KC-9′ (KSDSWHYWC; SEQ ID NO:26) and KC-10′ (KTPSPFDSHC; SEQ ID NO:25) were synthesized as analogs (Li, et al., Journal of Controlled Release, 148(3):292-302 (2010); Qin, et al., J Biochem, 142(1):79-85 (2007)). OPA-mediated cyclization followed by one-pot N-maleimide conjugation smoothly afforded the cCK-9′-fluorophore conjugate and other fluorophore conjugate analogs.

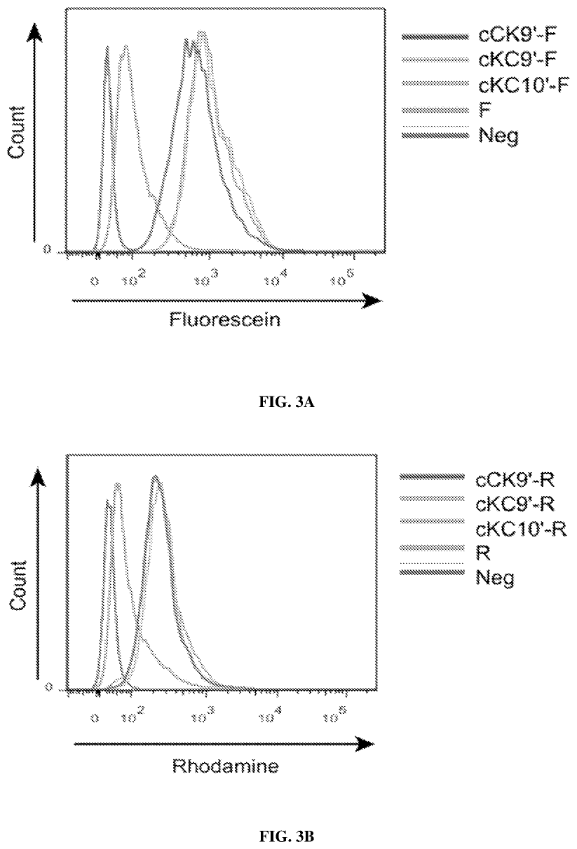

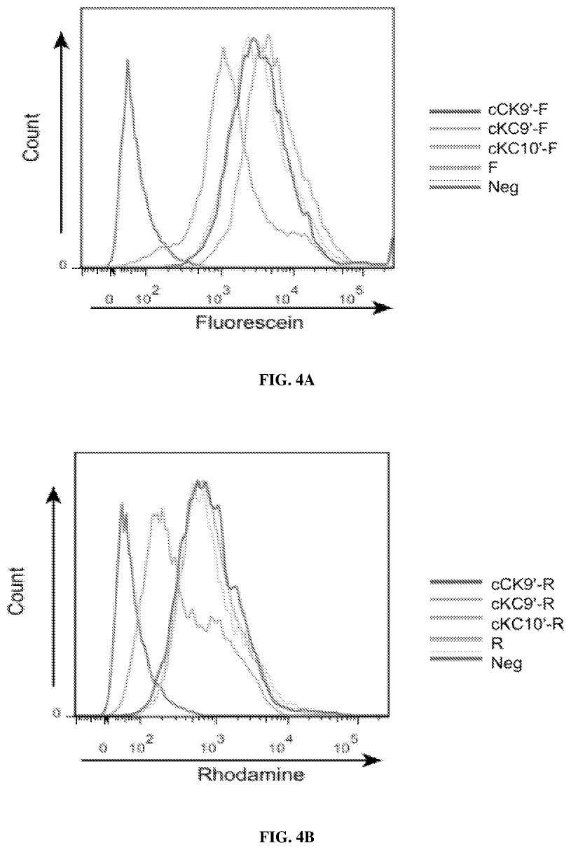

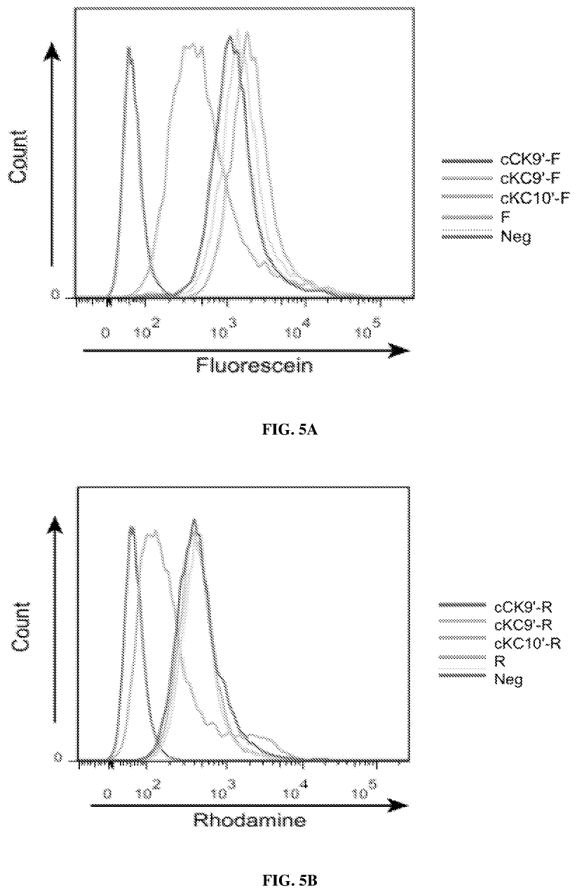

Next, fluorescence confocal imaging was employed to prove the specificity of the fluorophore conjugated cyclic peptide cCK-9′. The imaging of Caco2 cells with fluorescein conjugated cyclic peptide cCK-9′ was overlapped very well with cell membrane dye (DiI). How cytometry was also used to test the targeting specificity of fluorophores conjugated cyclic peptides (See Table 5 and FIGS. 3-6). Both fluorescein and rhodamine conjugated cyclic peptides showed specific binding to Caco2 cells, as compared to HT116 and A431 cells, which is in accordance with previous reports (Kelly, et al., Neoplasia, 5(5):437-444 (2003); Li, et al., Journal of Controlled Release, 148(3):292-302 (2010); Qin, et al., J. Biochem., 142(1):79-85 (2007)). These studies demonstrated that the OPA-mediated cyclization could effectively mimic the disulfide cyclic linkage, maintaining the cell targeting ability.

In all, the cyclic peptides from the OPA-mediated cyclization can be further modified with functional groups, such as fluorophores. Via easily changing the functionalized groups and recognition groups, various functional cyclic peptide biomolecules can be synthesized by this robust OPA cyclization guided post-modification.

TABLE 5
Peptide sequences and synthetic yields

| | | One-pot | | | |
|---|---|---|---|---|---|
| | | reaction | | | |
| | | conversion ^{[a]} | | HPLC Yield | |
| Name | Peptides | —F^{[b]} | —R^{[c]} | —F^{[b]} | —R^{[c]} |
| cKC10′ | Ac-KTPSPF | 86.9% | 74.4% | 45.01% | 62.67% |
| | DSHC-CONH₂ | | | | |
| | (SEQ ID | | | | |
| | NO: 25) | | | | |
| cKC9′ | Ac-KSDSWH | 99% | 86.9% | 32.82% | 30.66% |
| | YWC-CONH₂ | | | | |
| | (SEQ ID | | | | |
| | NO: 26) | | | | |
| cCK9′ | Ac-CPIEDR | 99% | 90.05% | 65.06% | 33.85% |
| | PMK-CONH₂ | | | | |
| | (SEQ ID | | | | |
| NO: 27) | | | | | |

^{[a]} conversion percentage was calculated based on LCMS profile
^{[b]}—F: Fluorescein
^{[c]}—R: Rhodamine

### Example 8. OPA Cyclization Guided Post-Modification Generates Various Useful Bioconjugates in Around 30 Minutes in One-Pot without Purification

Scheme of the OPA-mediated one-pot cyclization and bioconjugation reactions:

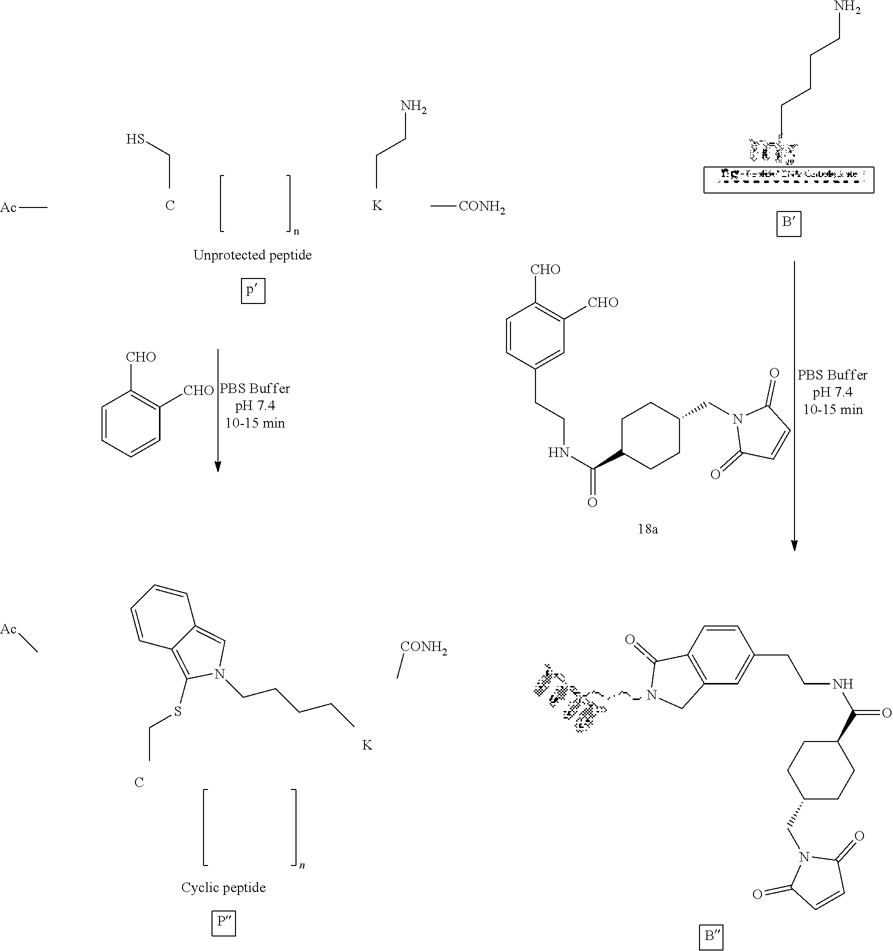

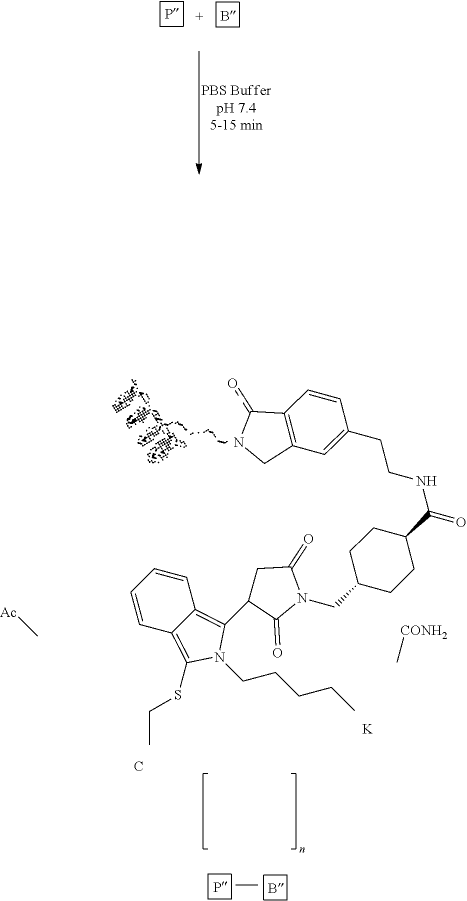

Materials and Methods

OPA Cyclization of Unprotected Peptides

The unprotected peptide (P′) (with a lysine and cysteine, 1 equiv.) was dissolved in PBS buffer (pH=7.4) with a final concentration of 0.2-0.5 mM. The OPA (ortho-phthalaldehyde) (1.05 equiv.) in DMSO was slowly added and the mixture was stirred at room temperature for 10-15 min.

Derivatized N-Maleimide with Various Functional Molecules (B″)

The functional molecules (B′) (peptide/DNA/carbohydrate, with a free amine, 1 equiv.) was dissolved in PBS buffer (pH=7.4) with a final concentration of 0.2-0.5 mM. The OPA-maleimide analog, N-(3,4-diformylphenethyl)-4-((2,5-dioxo-2,5-dihydro-1H-pyrrol-1-yl) methyl) cyclohexane-1-carboxamide (18a) (1.01 equiv.) was dissolved in DMSO, and directly added into the reaction mixture. The reaction was stirred at room temperature for 10-15 min.

### Synthesis of N-(3,4-diformylphenethyl)-4-((2,5-dioxo-2,5-dihydro-1H-pyrrol-1-yl) methyl) cyclohexane-1-carboxamide (18a)

Schemes of the synthesis of 18a:

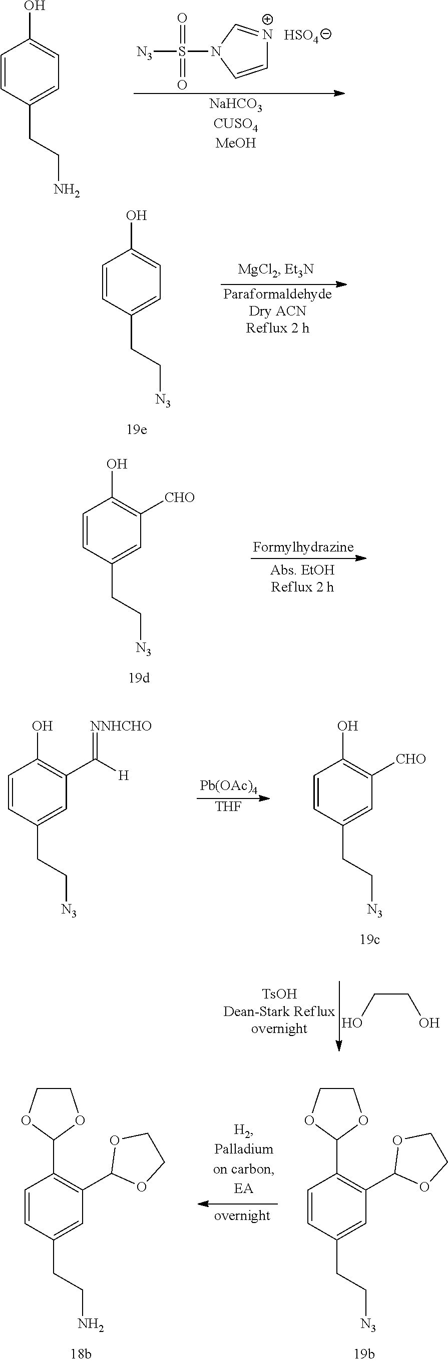

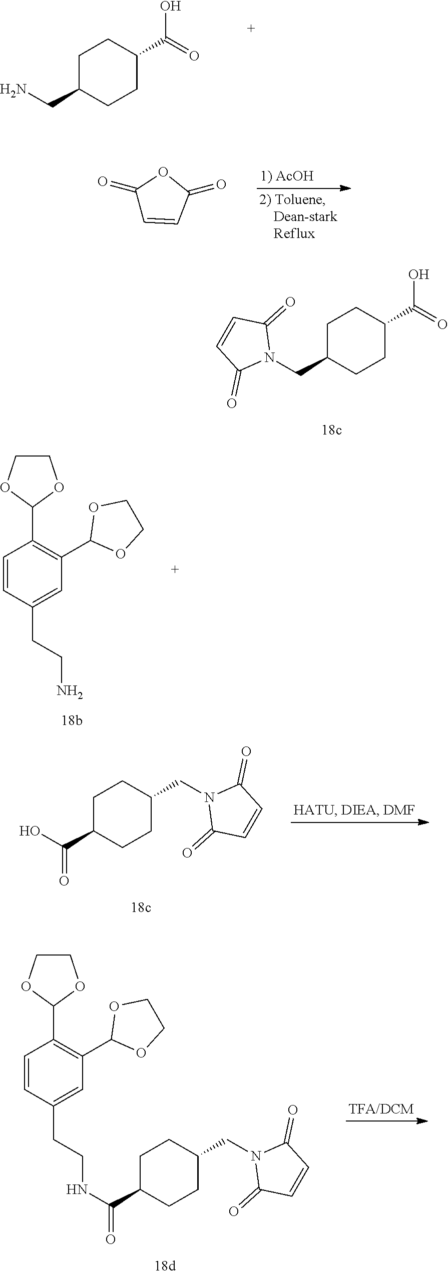 [SMILES: NC[C@H]1CC[C@H](C(=O)O)CC1 ; O=C1C=CC(=O)O1 ; O=C1C=CC(=O)N1C[C@H]1CC[C@H](C(=O)O)CC1 ; NCCc1ccc(C2OCCO2)c(C2OCCO2)c1 ; O=C1C=CC(=O)N1C[C@H]1CC[C@H](C(=O)O)CC1 ; O=C1C=CC(=O)N1C[C@H]1CC[C@H](C(=O)NCCc2ccc(C3OCCO3)c(C3OCCO3)c2)CC1]

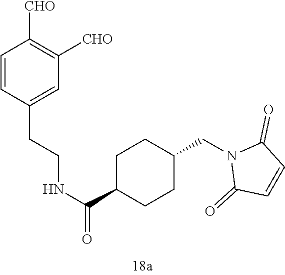 [SMILES: O=Cc1ccc(CCNC(=O)[C@H]2CC[C@H](CN3C(=O)C=CC3=O)CC2)cc1C=O]

### Synthesis of 4-(2-azidoethyl) phenol (19e)

To a stirred solution of tyramine (1.5 g, 10.9 mmol) and sodium bicarbonate in anhydrous MeOH (30 mL), imidazole-1-sulfonyl azide hydrogen sulfate (8.48 g, 31.9 mmol) was added into the mixture at room temperature followed by CuSO₄.5H₂O (7.4 mg, 0.03 mmol). The mixture was stirred at room temperature for overnight. The reaction was monitored by TLC plate and fully converted, the mixture was concentrated, diluted with water (50 mL), acidified with 1 N HCl solution and extracted twice with EtOAc. The combined organic layers were washed with brine, dried with anhydrous sodium sulfate and evaporated. The residue was purified by flash column chromatography on silica gel (EtOAc, 0.1% AcOH) to give compound 19e as yellow oil (1.39 g, 78.3%) (Goddard-Borger, et al., Organic letters., 9(19):3797-3800 (2007); Battersby, et al., Journal of the Chemical Society, Perkin Transactions., 1:31-42 (1980); Yang, et al., Journal of the American Chemical Society., 132(11):3640-3641 (2010)).

¹H NMR (500 MHz, CDCl₃) δ=7.10 (d, J=8.5 Hz, 2H), 6.80 (d, J=8.6 Hz, 2H), 5.65 (s, 1H), 3.47 (t, J=7.2 Hz, 2H), 2.84 (t, J=7.2 Hz, 2H) ppm. ¹³C NMR (126 MHz, CDCl₃) δ=154.11, 130.12, 129.89, 129.84, 115.48, 115.45, 52.57, 34.32.

### Synthesis of 5-(2-azidoethyl)-2-hydroxybenzaldehyde (19d)

To a stirred solution of compound 19e (1.39 g, 8.5 mmol), anhydrous magnesium dichloride (1.2 g, 12.6 mmol) and Et₃N (5.45 mL, 39.2 mmol) in anhydrous CH₃CN (70 mL), paraformaldehyde (1.73 g, 57.6 mmol) was added. The reaction mixture was heated under reflux for 3 h and cooled to room temperature. Then acidified by 1N HCl solution and extracted twice with EtOAc (200 mL). The combined organic layer was dried over anhydrous sodium sulfate, filtered, and evaporated. The residue was purified by flash column chromatography on silica gel (Hexane/EtOAc, 2:1 v/v) to give compound 19d as colorless oil (1.29 g, 79.0%).

¹H NMR (500 MHz, CDCl₃) δ=10.92 (s, 1H), 9.87 (d, J=0.6 Hz, 1H), 7.56-7.32 (m, 2H), 6.95 (d, J=8.3 Hz, 1H), 3.51 (t, J=7.0 Hz, 2H), 2.86 (t, J=7.0 Hz, 2H) ppm. ¹³C NMR (126 MHz, CDCl₃) δ=196.36, 160.38, 137.43, 133.45, 129.50, 120.41, 117.87, 52.21, 34.08.

### Synthesis of 4-(2-azidoethyl) phthalaldehyde (19c)

To a stirred solution of compound 19d (1.29 g, 6.74 mmol) in EtOH (90 mL), formic hydrazide (809 mg, 13.4 mmol) in EtOH (30 mL) was slowly added. The reaction mixture was refluxed for 2 h. The reaction was monitored by TLC plate and fully converted, the reaction mixture was cooled in ice bath and the precipitate was filtered and washed with hexane and ice cold EtOH. The residue was dried under vacuo for overnight. To a stirred solution of residue in anhydrous THF (120 mL), the lead(IV) acetate (6 g, 13.5 mmol) was added. The reaction mixture was then stirred at room temperature for 2.5 h. The reaction was monitored by TLC plate and fully converted, the mixture was filtered through celite, and the filtrate was concentrated under vacuum to give the crude aldehyde. The crude was dissolved in EtOAc (500 mL) and washed by brine. The organic layer was dried over anhydrous sodium sulfate, filtered, and evaporated. The residue was purified by flash column chromatography on silica gel (Hexane/EtOAc, 3;1 v/v) to give compound 19c as yellow solid (505 mg, 42.1%).

¹H NMR (500 MHz, CDCl₃) δ=10.52 (s, 1H), 10.43 (s, 1H), 7.90 (d, J=7.8 Hz, 1H), 7.80 (d, J=1.8 Hz, 1H), 7.62 (dd, J=7.8, 1.8 Hz, 1H), 3.59 (t, J=6.8 Hz, 2H), 3.00 (t, J=6.9 Hz, 2H) ppm. ¹³C NMR (126 MHz, CDCl₃) δ=192.11, 191.85, 144.88, 136.45, 134.81, 133.99, 131.78, 130.92, 51.36, 35.01.

### Synthesis of 2,2′-(4-(2-azidoethyl)-1,2-phenylene)bis(1,3-dioxolane) (19b)

To a stirred solution of compound 19c (2.56 g, 12.61 mmol) in anhydrous toluene (60 mL), p-toluenesulfonic acid (76 mg, 0.441 mmol) and ethylene glycol (7 mL, 125.1 mmol) were added. The mixture was refluxed in a dean-stark apparatus for 10 h. After the mixture was cooled down to room temperature, the reaction was quenched by Et₃N (0.5 mL, 3.58 mmol). The mixture was then evaporated under vacuo, and the residue was dissolved in EtOAc. The organic layers were washed with Sat. NaHCO₃(aq.) and brine, dried with anhydrous sodium sulfate and evaporated. The residue was purified by flash column chromatography on silica gel (Hexane/EtOAc, 3:1 v/v) to give compound 19b as light-yellow oil (2.77 g, 75.68%).

¹H NMR (400 MHz, CDCl₃) δ=7.58 (d, J=7.9 Hz, 1H), 7.49 (d, J=1.9 Hz, 1H), 7.23 (dd, J=8.0, 1.9 Hz, 1H), 6.22 (s, 2H), 6.19 (s, 2H), 4.24-4.04 (m, 4H), 4.08-3.96 (m, 4H), 3.48 (t, J=7.4 Hz, 2H), 2.89 (t, J=7.4 Hz, 2H). ¹³C NMR (100 MHz, CDCl₃) δ=138.85, 136.21, 134.59, 129.27, 126.42, 126.27, 126.24, 100.53, 100.46, 65.19, 65.17, 52.07, 34.99.

### Synthesis of 2-(3,4-di(1,3-dioxolan-2-yl)phenyl)ethan-1-amine (18b)

To a stirred solution of compound 19b (1.25 g, 4.25 mmol) in a 25 mL round bottom flask, palladium on carbon (10% w/w, 200 mg) and EtOAc (8 mL) was added. The mixture was stirred under 1.1 atm H₂ atmosphere until the hydrogenation was completed, then was filtered through celite to remove the catalyst. The solvent was concentrated under vacuum and the product 18b (1.007 g, 89.411%) was obtained as light-yellow oil.

¹H NMR (500 MHz, CDCl₃) δ=7.56 (d, J=7.9 Hz, 1H), 7.47 (d, J=1.9 Hz, 1H), 7.22 (dd, J=7.9, 1.9 Hz, 1H), 6.18 (d, J=2.1 Hz, 2H), 4.18-4.08 (m, 4H), 4.07-3.98 (m, 4H), 2.95 (t, J=6.9 Hz, 2H), 2.77 (t, J=6.9 Hz, 2H) ppm. ¹³C NMR (126 MHz, CDCl₃) δ=140.66, 135.98, 134.02, 129.49, 126.41, 126.34, 100.67, 100.65, 65.26, 65.24, 43.19, 39.55.

### Synthesis of trans-4-(maleimidomethyl)cyclohexanecarboxylic acid (18c)

The trans-4-(Maleimidomethyl)cyclohexanecarboxylic acid was synthesized by following the literature (Pieczykol, et al., WO2014141094A1).

### Synthesis of N-(3,4-di(1,3-dioxolan-2-yl)phenethyl)-4-((2,5-dioxo-2,5-dihydro-1H-pyrrol-1-yl)methyl)cyclohexane-1-carboxamide (18d)

To a stirred solution of compound 18c (250 mg, 1.05 mmol) in anhydrous DMF (15 mL), 1-[bis(dimethylamino)methylene]-1H-1,2,3-triazolo[4,5-b]pyridinium 3-oxid hexafluorophosphate (HATU) (400 mg, 1.05 mmol), DIPEA (0.44 mL, 2.1 mmol), compound 18b (420 mg, 1.58 mmol) were added and the reaction was stirred at room temperature for overnight. The reaction was monitored by TLC plate and fully converted, the solvent was removed by vacuo. The residue was dissolved in EtOAc, then washed by 0.5 N HCl solution and brine. The organic layer was dried with anhydrous sodium sulfate and evaporated. The residue was purified by flash column chromatography on silica gel (Hexane/EtOAc, 1:3 v/v) to give compound 18d as white solid (450 mg, 88.1%).

¹H NMR (500 MHz, CDCl₃) δ=7.65 (d, J=10 Hz, 1H), 7.49 (d, J=10 Hz, 1H), 7.37 (s, 1H), 7.13-7.12 (m, 1H), 6.63 (s, 2H), 6.08 (s, 2H), 4.08-4.06 (m, 4H), 3.99-3.97 (m, 4H), 3.375 (d, J=5 Hz, 2H), 3.27 (d, J=5 Hz, 2H), 2.87 (s, 1H), 1.88 (m, 1H), 1.75 (d, J=10 Hz, 2H), 1.64 (d, J=10 Hz, 2H), 1.36-1.31 (m, 2H), 1.19 (m, 2H), 0.88 (d, J=15 Hz, 2H) ppm. ¹³C NMR (126 MHz, CDCl₃) δ=176.14, 176.06, 170.98, 140.04, 135.77, 133.83, 133.46, 130.20, 129.43, 126.39, 100.47, 100.29, 65.13, 65.10, 49.25, 49.08, 48.90, 44.47, 43.45, 39.99, 38.35, 36.16, 35.07, 29.60, 28.50, 27.24 ppm.

### Synthesis of N-(3,4-diformylphenethyl)-4-((2,5-dioxo-2,5-dihydro-1H-pyrrol-1-yl) methyl) cyclohexane-1-carboxamide (18a)

To a solution of compound 18d (450 mg, 0.92 mmol) in DCM (2 mL), TFA (8 mL) was slowly added during 0° C. The reaction was stirred at room temperature for 3 h. The mixture was then evaporated under vacuo, and the residue was purified by flash column chromatography on silica gel (DCM/EtOAc, 1:3 v/v) to give compound 18a as white solid (312 mg, 84.7%).

¹H NMR (500 MHz, CD₃CN) δ=10.43 (s, 1H), 10.39 (s, 1H), 7.89 (d, J=7.8 Hz, 1H), 7.77 (d, J=1.6 Hz, 1H), 7.64 (dd, J=7.8, 1.8 Hz, 1H), 6.74 (s, 2H), 3.51-3.34 (m, 2H), 2.89 (t, J=6.9 Hz, 2H), 1.95 (td, J=5.2, 2.7 Hz, 2H), 1.64 (td, J=14.1, 3.2 Hz, 4H), 1.26-1.19 (m, 2H), 0.94-0.84 (m, 2H). ¹³C NMR (126 MHz, CD₃CN) δ=193.41, 193.02, 176.78, 171.66, 146.51, 136.42, 134.58, 134.25, 134.02, 130.93, 44.42, 43.19, 39.26, 36.20, 34.83, 29.32, 28.45.

One Pot OPA Cyclization and Followed Bioconjugation with Various Biomolecules

The (bi)cyclo-peptides (P″) obtained from OPA cyclization of unprotected peptides (P′) and the derivatized N-maleimide (B″) with various functional molecules (B′) were directed mixed together at the same ratio (P″:B″=1:1 mol/mol) at room temperature for 5-15 min. The bioconjugation reaction was monitored by LC-MS. After the reaction, the mixture was directly diluted by H₂O/ACN (with 0.1% TFA) and monitor by LC-MS.

(SEQ ID NO: 15, SEQ ID NO: 11)
Cyclo-(Ac-KAAAAACH-CONH₂)
(Ac-AFAQK-CONH₂)

The bioconjugation reaction between cyclo-(Ac-KAAAAACH-CONH₂) (SEQ ID NO:15) and (Ac-AFAQK-CONH₂; SEQ ID NO:11) provides compound 20a. UV trace from LC-MS analysis of the reaction shows the one-pot OPA-cyclization followed bioconjugation reaction give a clean major peak of conjugated product. Gradient: 5%-95% ACN/H₂O with 0.1% TFA over 10 min at a flow rate of 0.4 mL/min. ESI-MS calcd. for C₉₀H₁₂₂N₂₂O₂₀S [M+H]⁺ m/z=1865.16, [M+2H]²⁺ m/z=933.08, [M+3H]³⁺ m/z=622.33, found 622.47, 932.58.

(SEQ ID NO: 10, SEQ ID NO: 11)
Cyclo-(Ac-ENPECILDKHVQRVM-CONH₂)
(Ac-AFAQK-CONH₂)

The bioconjugation reaction between cyclo-(Ac-ENPECILDKHVQRVM-CONH₂) (SEQ ID NO:10) and (Ac-AFAQK-CONH₂; SEQ ID NO:11) provides compound 20b. UV trace from LC-MS analysis of the reaction shows the one-pot OPA-cyclization followed bioconjugation reaction give a clean major peak of conjugated product. Gradient: 5%-95% ACN/H₂O with 0.1% TFA over 10 min at a flow rate of 0.4 mL/min. ESI-MS calcd. for C₁₃₆H₁₉₈N₃₄O₃₅S₂ [M+H]⁺ m/z=2934.4, [M+2H]²⁺ m/z=1467.7, [M+3H]³⁺ nm/z=978.8, found 978.72, 1467.30.

(SEQ ID NO: 14, SEQ ID NO: 11)
Bicyclo-(Ac-CNSTKNLTFAMRSSGDYGEV-
CONH₂)(Ac-AFAQK-CONH₂)

The bioconjugation reaction between bicyclo-(Ac-CNSTKNLTFAMRSSGDYGEV-CONH₂) (SEQ ID NO:14) and (Ac-AFAQK-CONH₂; SEQ ID NO:11) provides compound 20c. UV trace from LC-MS analysis of the reaction shows the one-pot OPA-cyclization followed bioconjugation reaction give a clean major peak of conjugated product. Gradient: 5%-95% ACN/H₂O with 0.1% TFA over 10 min at a flow rate of 0.4 m/min. ESI-MS calcd. for C₁₄₈H₂₀₈N₃₆O₄₃S₂ [M+H]⁺ m/z=3242.46, [M+2H]²⁺ m/z=1621.23, [M+3H]³⁺ nm/z=1081.48, found 1082.43, 1622.57.

(SEQ ID NO: 10)
Cyclo-(Ac-ENPECILDKHVQRVM-CONH₂)
(D-(+)-Glucosamine)

The bioconjugation reaction between cyclo-(Ac-ENPECILDKHVQRVM-CONH₂) (SEQ ID NO:10) and (D-(+)-Glucosamine) provides compound 20d. UV trace from LC-MS analysis of the reaction shows the one-pot OPA-cyclization followed bioconjugation reaction give a clean major peak of conjugated product. Gradient: 5%-95% ACN/H₂O with 0.1% TFA over 10 min at a flow rate of 0.4 mL/min. ESI-MS calcd. for C₁₁₄H₁₆₇N₂₇O₃₃S₂[M+H]⁺ m/z=2508.86, [M+2H]²⁺ m/z=1254.43, [M+3H]³⁺ m/z=837.62, found 837.09, 1255.39.

(SEQ ID NO: 10, SEQ ID NO: 28)
Cyclo-(Ac-ENPECILDKHVQRVM-CONH₂)
(5′-NH₂-C6-ATCGATCGATCG-3′)

The bioconjugation reaction between cyclo-(Ac-ENPECILDKHVQRVM-CONH₂) (SEQ ID NO:10) and (5′-NH₂—C6-ATCGATCGATCG-3′; SEQ ID NO:28) provides compound 20e. UV trace from LC-MS analysis of the reaction shows the one-pot OPA-cyclization followed bioconjugation reaction give a clean major peak of conjugated product. Gradient: 0%-35% ACN/H₂O with 0.1% TFA over 10 min at a flow rate of 0.4 mL/min. MALDI-TOF spectrum of the reaction shows a clean mass result of conjugated product.

Results

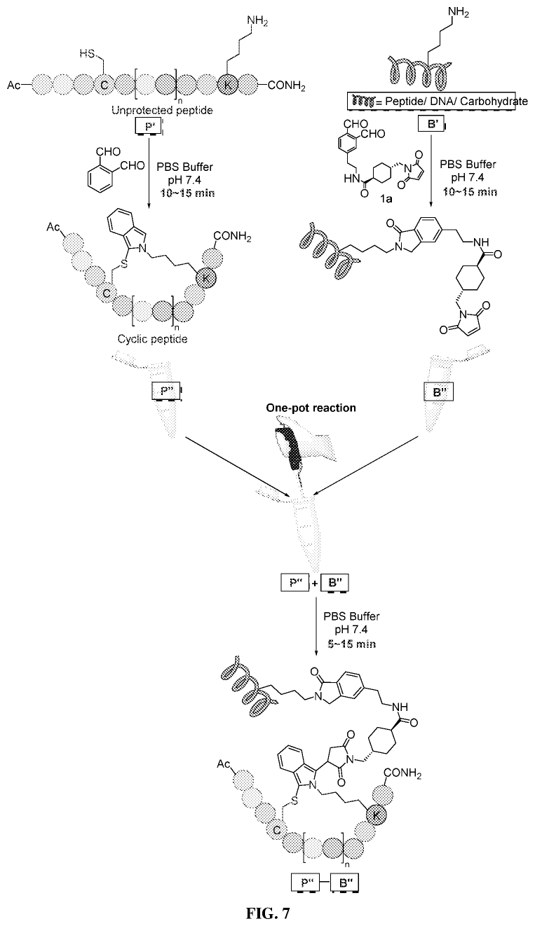

N-maleimide was derivatized with various functional molecules including glycans, peptides, and amine modified DNA, which could be subsequently introduced onto the cyclic peptides, providing useful bioconjugates (See FIG. 7, compounds 20a-20e described above and Table 6). Towards this end, an N-maleimide-OPA bifunctional linker was designed, which could readily react with an amine group present on the functional molecule B′ (e.g., glycan, peptide or DNA) via phthalimidine chemistry as previously developed to afford conjugates B″. The resultant conjugate B″ was subsequently reacted with OPA cyclic peptides P″ in one-pot manner to generate various cyclic peptide-peptide, cyclic peptide-glycan and cyclic peptide-DNA hybrid compounds. All examples tested could be completed cleanly in around 30 minutes in one-pot manner without any purification steps (See compounds 20a-20e described above and Table 6).

TABLE 6
Examples of the OPA-mediated one-pot
cyclization and bioconjugation.

| | P′ + OPA | | B′ + 18a | | P″ + B″ |
|---|---|---|---|---|---|
| No. | P′^{[a]} | Time | B′ | Time | Time^{[c]} |
| 20a | Ac-KAAAAA | 15 min | Ac-AFAQK- | 15 min | 10 min |
| | CH-CONH₂ | | CONH₂^{[a]} | | |
| | (SEQ ID | | (SEQ ID | | |
| | NO: 15) | | NO: 11) | | |
| 20b | Ac-ENPECI | 15 min | Ac-AFAQK- | 15 min | 5 min |
| | LDKHVQRVM- | | CONH₂ | | |
| | CONH₂ | | (SEQ ID | | |
| | (SEQ ID | | NO: 11) | | |
| | NO: 10) | | | | |
| 20c | Cyclo- | 15 min | Ac-AFAQK- | 15 min | 15 min |
| | (CNSTKNLT | | CONH₂ | | |
| | FAMRSSGDY | | (SEQ ID | | |
| | GEV) | | NO: 11) | | |
| | (SEQ ID | | | | |
| | NO: 14) | | | | |
| 20d | Ac-ENPEC | 15 min | Glucosamine^{[a]} | 15 min | 15 min |
| | ILDKHVQR | | | | |
| | VM- | | | | |
| | CONH₂ | | | | |
| | (SEQ ID | | | | |
| | NO: 10) | | | | |
| 20e | Ac-ENPEC | 15 min | 5′-NH₂-C₆- | 15 min | 15 min |
| | ILDKHVQR | | ATCGATCGA | | |
| | VM- | | TCG^{[b]} | | |
| | CONH₂ | | (SEQ ID | | |
| | (SEQ ID | | NO: 28) | | |
| | NO: 10) | | | | |

^{[a]}Reaction concentration is 0.5 mM.
^{[b]}Reaction concentration is 0.2 mM.
^{[b]}P″:B″ = 1:1 mol/mol.

### Example 9. TDA-Cyclization Provide a Simple and Chemoselective Way to Cyclize Peptides

Materials and Methods

Scheme of the chemoselective TDA-cyclization reaction:

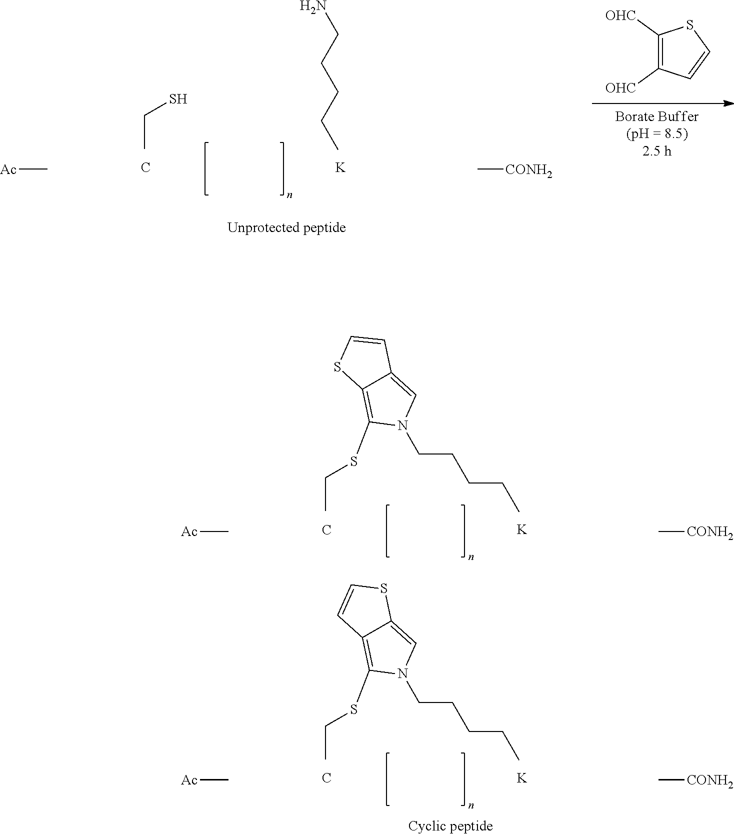

Solid Phase Peptide Synthesis

Synthesis was performed manually on rink amide resin (GL Biochem) under the standard Fmoc-SPPS protocol. Removal of Fmoc protecting group was performed using a mixture of 20/80 (v/v) of piperidine/DMF for 15-20 min. Coupling was performed using Fmoc-amino acids (4.0 equiv.), HATU (4.0 equiv.) and DIPEA (8.0 equiv.) in DMF for 1 hour at room temperature. For N-terminal acetylated peptide, anhydrous CH₂Cl₂: Pyridine: Acetic anhydride (2:1:1, v:v:v) with resin for 1 h at room temperature. Upon completion of the synthesis, 9.5:0.25:0.25 of TFA: TIPS: Water (v:v:v) were used to perform global deprotection. The peptides were then precipitated in cold diethyl ether and purified by preparative RP-HPLC.

TDA-Cyclization with Model Peptides

Model peptides (with a lysine and cysteine, 1 equiv.) were dissolved in Borate buffer (pH=8.5) with a final concentration of 0.5 mM. 2,3-Thiophenedicarboxaldehyde (1.1-1.4 equiv.) in DMSO was added to the solution and the reaction was stirred at room temperature around 2-2.5 h. The conversion was monitored by LC-MS.

Results

Eighteen model peptides (including bicyclic peptides) with different length and spacing amino acids of 2 to 7 residues between Lys side chain and the Cys residue reacted with TDA in the Borate buffer (pH=8.5) to afford cyclic peptides (or bicyclic peptides) of different rings, with 88->98% conversions judged by LC-MS analysis of the crude reaction mixtures (See compounds 23a-23r and 23a′-23r′ described above and Table 7). Various side chain functionalities present in the unprotect peptides did not interfere the reaction. Thus, this chemoselective TDA-cyclization provided a simple way to cyclize unprotected native peptides. It should be pointed out that this reaction can differentiate the side chain amino group and the N-terminal amine, thus only Lys side-chain can produce clan side chain-to-side chain cyclic peptide. The N-terminal has extremely low reactivity in the same condition in TDA-cyclization manner.

TABLE 7
TDA-guided cyclization with different peptide sequences.

| No. | Sequence | Cal. MS | Obtained MS | Conversion^{[a]} |
|---|---|---|---|---|
| 23a | Ac-KAAACH- | [M + H]⁺ = 745.30 | 745.31 | 94% |
| and | CONH₂ | | | |
| 23a′ | (SEQ ID | | | |
| | NO: 16) | | | |
| 23b | Ac-KAAAACH- | [M + H]⁺ = 816.05 | 816.17 | 97% |
| and | CONH₂ | | | |
| 23b′ | (SEQ ID | | | |
| | NO: 15) | | | |
| 23c | Ac-KAAAAACH- | [M + H]⁺ = 886.12 | 887.20 | 96% |
| and | CONH₂ | | | |
| 23c′ | (SEQ ID | | | |
| | NO: 17) | | | |
| 23d | Ac-KAAAAAACH- | [M + H]⁺ = 957.00 | 958.40 | 90% |
| and | CONH₂ | | | |
| 23d′ | (SEQ ID | | | |
| | NO: 29) | | | |
| 23e | Ac- | [M + H]⁺ = 905.33 | 906.34 | 97% |
| and | CDWLPK- | | | |
| 23e′ | CONH₂ | | | |
| | (SEQ ID | | | |
| | NO: 19) | | | |
| 23f | Ac-ENCPEI | [M + 2H]²⁺ = 979.10 | 979.14 | >98% |
| and | LDKHVQRVM- | | | |
| 23f | CONH₂ | | | |
| | (SEQ ID | | | |
| | NO: 30) | | | |
| 23g | Ac-ACFALPKG- | [M + H]⁺ = 951.63 | 951.63 | >98% |
| and | CONH₂ | | | |
| 23g′ | (SEQ ID | | | |
| | NO: 20) | | | |
| 23h | Ac-KGHAFQC- | [M + H]⁺ = 927.23 | 927.50 | 95% |
| and | CONH₂ | | | |
| 23h′ | (SEQ ID | | | |
| | NO: 21) | | | |
| 23i | Ac-GAQCAFLK- | [M + H]⁺ = 982.19 | 982.36 | >98% |
| and | CONCONH₂ | | | |
| 23i′ | (SEQ ID | | | |
| | NO: 22) | | | |
| 23j | Ac-AKVTMTCSAS- | [M + H]⁺ = 1143.20 | 1143.21 | 95% |
| and | CONH₂ | | | |
| 23j′ | (SEQ ID | | | |
| | NO: 23) | | | |
| 23k | Ac-KTPSPFDSHC- | [M + 2H]²⁺ = 632.29 | 632.46 | 97% |
| and | CONH₂ | | | |
| 23k′ | (SEQ ID | | | |
| | NO: 25) | | | |
| 23l | Ac-KSDSWHYWC- | [M + 2H]²⁺ = 678.94 | 679.11 | 91% |
| and | CONH₂ | | | |
| 23l′ | (SEQ ID | | | |
| | NO: 26) | | | |
| 23m | Ac-CPIED | [M + 2H]²⁺ = 617.52 | 617.56 | >98% |
| and | RPMK- | | | |
| 23m′ | CONH₂ | | | |
| | (SEQ ID | | | |
| | NO: 27) | | | |
| 23n | cyclo- | [M + 2H]²⁺ = 1133.99 | 1134.24 | 88% |
| and | (CNSTKNL | | | |
| 23n′ | TFAMRSSG | | | |
| | DYGEV) | | | |
| | (SEQ ID | | | |
| | NO: 14) | | | |
| 23o | cyclo- | [M + H]⁺ = 779.41 | 779.52 | >98% |
| and | (Dap-Glu- | | | |
| 23o′ | Ala-(D) | | | |
| | Thr-Cys- | | | |
| | Hyp) | | | |
| | (SEQ ID | | | |
| | NOG 1) | | | |
| 23p | Ac-ENPEC | [M + 2H]²⁺ = 979.10 | 979.31 | >98% |
| and | IEDKHVQR | | | |
| 23p′ | VM- | | | |
| | CONH₂ | | | |
| | (SEQ ID | | | |
| | NO:10) | | | |
| 23q | Ac-CENPEI | [M + 2H]²⁺ = 979.10 | 979.06 | 96% |
| and | LDHVKQRVM- | | | |
| 23q′ | CONH₂ | | | |
| | (SEQ ID | | | |
| | NO: 32) | | | |
| 23r | Ac-CENPEI | [M + 2H]²⁺ = 979.10 | 979.14 | 97% |
| and | LDKHVQRVM- | | | |
| 23r′ | CONH₂ | | | |
| | (SEQ ID | | | |
| | NO: 33) | | | |

^{[a]}conversion percentage was calculated based on LCMS profile.

The Examples have demonstrated that OPA- and TDA-amine-thiol three-component reaction can be effectively used for the synthesis of novel cyclic peptide motifs, directly using unprotected peptides as the starting material. The OPA cyclization and TDA cyclization has demonstrated high chemoselectivity under mild conditions. Furthermore, by integrating NCL and OPA- or TDA-mediated chemoselective peptide cyclization, bi-cyclic peptides were easily prepared. The cyclic peptide product from OPA and/or TDA cyclization can be further modified with DMAC or N-maleimide derivatives for constructing new architecture and incorporating functional moieties. In this regard, N-maleimide-OPA and N-maleimide-TDA bifunctional linkers offer a simple way to conjugate amine-containing biomolecules to the cyclic peptide obtained from OPA and TDA cyclization respectively. Overall, the OPA cyclization and TDA cyclization methods can be applied for the synthesis of various functional cyclic/bicyclic peptides, peptide conjugates and branched peptides in both chemical biology study and drug discovery. The operational simplicity and high efficiency of OPA peptide cyclization and TDA peptide cyclization also provides a new tool for construction of DNA-encoded cyclic peptide library.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A compound having a structure of Formula I: 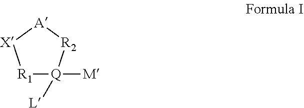
(a) wherein A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;
(b) wherein X′ is —NR₃, an oxygen atom, or a sulfur atom, wherein R3 is a hydrogen, a substituted alkyl group, a substituted cycloalkyl group, a substituted heteroalkyl group, a substituted alkenyl group, a substituted heteroalkenyl group, a substituted aryl group, or a substituted heteroaryl group;
(c) wherein R₁ and R₂ are independently absent, an unsubstituted alkyl group, a substituted alkyl group, an unsubstituted heteroalkyl group, or a substituted heteroalkyl group;
(d) wherein Q is an oligomer or a synthetic material; and
(e) wherein L′ and M′ are independently absent, one or more monomer residues or a synthetic material,
(f) provided that when Formula I has the structure of Formula III: 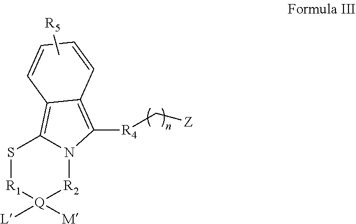
wherein R₄ is hydrogen, n is 0, and Z is absent,
then:
(i) R₅ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group, an unsubstituted carbonyl group,
an acyl group optionally containing a substituted alkenyl group, a substituted heteroalkenyl group, or a substituted heteroaryl group,
an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or
a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group; or
(ii) R₅ is a substituted carbonyl group, wherein the substituent is —NRT2′(C═O)NRT3′-, RT2′ is an alkyl group, and RT3′ is hydrogen atom, a halogen atom, an alkyl group, a heteroalkyl group, an alkenyl group, a heteroalkenyl group, an alkynyl group, a heteroalkynyl group, an aryl group, or a heteroaryl group; or
(iii) R₅ is an acyl group, wherein the acyl group does not contain a substituted alkyl group, a substituted cycloalkyl group, a substituted heteroalkyl group, or a substituted aryl group.

2. The compound of claim 1, wherein the monomer residues are independently amino acid residues or nucleotide residues.

3. The compound of claim 1, wherein Q is a peptide or an oligonucleotide.

4. The compound of claim 1 having a structure of Formula II: 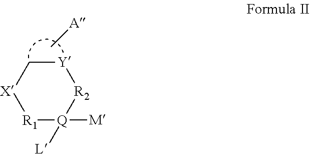
(a) wherein X′, R₁, R₂, Q, L′, and M′ are as defined in the base claim(s);
(b) wherein A″ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;
(c) wherein Y′ is a nitrogen atom.

5. The compound of claim 4, wherein A″ is an unsubstituted polyaryl group, a substituted polyaryl group, an unsubstituted polyheteroaryl group, or a substituted polyheteroaryl group.

6. The compound of claim 1, wherein X′ is a sulfur atom.

7. The compound of claim 1, wherein Q is a peptide.

8. The compound of claim 7, wherein the peptide is a linear peptide, a cyclic peptide, or a branched peptide.

9. The compound of claim 1, wherein Q is an unprotected peptide.

10. The compound of claim 1, wherein L′ and M′ are independently one or more amino acid residues.

11. The compound of claim 1, wherein Q is an oligomer of synthetic monomer residues.

12. The compound of claim 1, wherein the compound is fluorescent.

13. The compound of claim 1 having a structure of Formula III: 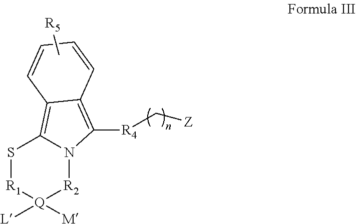
(a) wherein R₁, R₂, Q, L′, and M′ are as defined in the base claim(s);
(b) wherein R₄ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group,
an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,
an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or
a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;
(c) wherein R₅ is a hydrogen, an unsubstituted alkenyl group, a substituted alkenyl group, an unsubstituted heteroalkenyl group, a substituted heteroalkenyl group, an unsubstituted succinimidyl group, a substituted succinimidyl group, an unsubstituted aryl group, a substituted aryl group, an unsubstituted heteroaryl group, a substituted heteroaryl group, an unsubstituted carbonyl group, a substituted carbonyl group,
an acyl group optionally containing a substituted alkyl group, a substituted cycloalkyl group, a substituted heteroalkyl group, a substituted alkenyl group, a substituted heteroalkenyl group, a substituted aryl group, or a substituted heteroaryl group,
an ester group optionally containing a substituted alkyl group, a substituted cycloalkyl group, a substituted heteroalkyl group, a substituted alkenyl group, a substituted heteroalkenyl group, a substituted aryl group, or a substituted heteroaryl group, or
a hydroxamate group optionally containing a substituted alkyl group, a substituted cycloalkyl group, a substituted heteroalkyl group, a substituted alkenyl group, a substituted heteroalkenyl group, a substituted aryl group, or a substituted heteroaryl group;
(d) wherein n is zero or a positive integer; and
(e) wherein Z is optional and comprises a chemical probe and/or a biofunctional molecule.

14. The compound of claim 1, having a structure of Formula III′ or Formula III″ 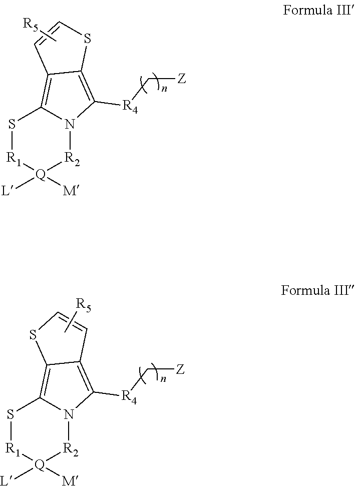
wherein R₁, R₂, R₄, R₅, Q, L′, M′, n and Z are as defined above.

15. The compound of claim 13, wherein when R₄ is hydrogen, n is zero, and Z is absent.

16. The compound of claim 13, wherein R₄ is an unsubstituted alkenyl group, a substituted alkenyl group, an unsubstituted heteroalkenyl group, or a substituted heteroalkenyl group.

17. The compound of claim 13, wherein R₄ is an unsubstituted succinimidyl group or a substituted succinimidyl group.

18. The compound of claim 13, wherein Z comprises a luminescence probe.

19. The compound of claim 18, wherein the luminescence probe is an organic dye, a biological fluorophore, or a quantum dot.

20. The compound of claim 19, wherein the luminescence probe is an organic dye selected from the group consisting of fluorescein, rhodamine, and derivatives thereof.

21. The compound of claim 13, wherein Z comprises a colorimetric probe.

22. The compound of claim 13, wherein Z comprises a biofunctional molecule selected from the group consisting of glycans, peptides, oligonucleotides, proteins, and small molecule drugs.

23. A method of making the compound of claim 1, comprising:
(a) performing a reaction between a compound of Formula IV and a compound of Formula V to form an adduct, 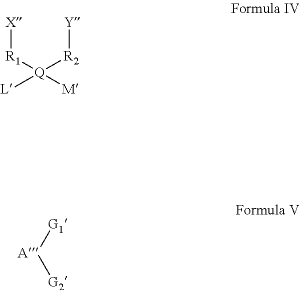
wherein R₁, R₂, Q, L′, and M′ are as defined in the base claim(s);
wherein X″ and Y″ are independently a carboxylic acid group, a carboxylate group,
an amino group optionally containing one substituent at the amino nitrogen, wherein the substituent is a substituted alkyl group, a substituted cycloalkyl group, a substituted heteroalkyl group, a substituted alkenyl group, a substituted heteroalkenyl group, a substituted aryl group, or a substituted heteroaryl group,
a hydroxyl group optionally containing one substituent at the hydroxyl oxygen, wherein the substituent is a substituted alkyl group, a substituted cycloalkyl group, a substituted heteroalkyl group, a substituted alkenyl group, a substituted heteroalkenyl group, a substituted aryl group, or a substituted heteroaryl group, or
a thiol group optionally containing one substituent at the thiol sulfur, wherein the substituent is a substituted alkyl group, a substituted cycloalkyl group, a substituted heteroalkyl group, a substituted alkenyl group, a substituted heteroalkenyl group, a substituted aryl group, or a substituted heteroaryl group;
wherein A′″ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group; and
wherein G1′ and G2′ are independently an aldehyde group, a cyanate groups, a nitrile group, an isonitrile group, a nitro group, a nitroso group, a nitrosooxy group, an acyl group, a carboxylic acid group, or a carboxylate group.

24. The method of claim 23, further comprising:
(b) performing a reaction between the adduct from step (a) and a reactant to form a second adduct, wherein the reactant is an unsubstituted maleimide, a substituted maleimide, an unsubstituted alkynyl group, a substituted alkynyl group, or derivatives thereof.

25. The method of claim 23, wherein the compound of Formula V is ortho-phthalaldehyde (OPA).

26. The method of claim 23, wherein the compound of Formula V is 2,3-Thiophenedicarboxaldehyde (TDA).

27. The method of claim 23, wherein X″ is a thiol group and Y″ is an amino group.

28. The method of claim 23, wherein the reaction is performed in a buffer solution.

29. The method of claim 28, wherein the buffer solution is selected from the group consisting of acetate buffer, phosphate buffer, HEPES buffer, TEAA buffer, and borate buffer.

30. The method of claim 23, wherein the reaction is performed at a pH of at least about 6, preferably at least about 7, more preferably at least about 7.4.

31. The method of claim 23, wherein the reaction is performed at a pH of at least about 8, preferably at least about 8.5.

32. The method of claim 23, wherein the reaction in step (a) is performed at a rate wherein at least 80% of the compound of Formula IV and/or of the compound of Formula V has reacted at about 2.5 hours, preferably at about 2 hours, more preferably at about 1.5 hours.

33. The method of claim 23, wherein the reaction in step (a) is performed at a rate wherein 80% of the compound of Formula IV and/or of the compound of Formula V has reacted at about 30 minutes, preferably at about 20 minutes, more preferably at about 10 minutes.

34. The method of claim 23, wherein the reaction in step (b) is performed at a rate wherein 80% of the adduct formed in step (a) and/or the reactant has reacted at about 30 minutes, preferably at about 20 minutes, more preferably at about 10 minutes.

35. The method of claim 23, wherein the reaction of step (a) reaches a conversion of at least about 70%, preferably at least about 80%, more preferably at least about 90%.

36. The method of claim 23, wherein the reaction of step (b) reaches a conversion of at least about 70%, preferably at least about 80%, more preferably at least about 90%.


SEQUENCE LISTING (37 sequences)

SEQ ID NO: 1 - protein, 5 aa, Artificial Sequence
  Synthetic Peptide
  MISC_FEATURE (5)..(5): amidated phenylalanine
AAACX

SEQ ID NO: 2 - protein, 6 aa, Artificial Sequence
  Synthetic Peptide
  MISC_FEATURE (6)..(6): amidated phenylalanine
AAAACX

SEQ ID NO: 3 - protein, 7 aa, Artificial Sequence
  Synthetic Peptide
  MISC_FEATURE (7)..(7): amidated phenylalanine
AAAAACX

SEQ ID NO: 4 - protein, 6 aa, Artificial Sequence
  Synthetic Peptide
  MISC_FEATURE (1)..(1): acetylated lysine
  MISC_FEATURE (6)..(6): amidated phenylalanine
XAAACX

SEQ ID NO: 5 - protein, 7 aa, Artificial Sequence
  Synthetic Peptide
  MISC_FEATURE (1)..(1): acetylated lysine
  MISC_FEATURE (7)..(7): amidated phenylalanine
XAAAACX

SEQ ID NO: 6 - protein, 8 aa, Artificial Sequence
  Synthetic Peptide
  MISC_FEATURE (1)..(1): acetylated lysine
  MISC_FEATURE (8)..(8): amidated phenylalanine
XAAAAACX

SEQ ID NO: 7 - protein, 9 aa, Artificial Sequence
  Synthetic Peptide
  MISC_FEATURE (1)..(1): acetylated lysine
  MISC_FEATURE (9)..(9): amidated phenylalanine
XAAAAAACX

SEQ ID NO: 8 - protein, 9 aa, Artificial Sequence
  Synthetic Peptide
  MISC_FEATURE (1)..(1): acetylated cysteine
  MISC_FEATURE (9)..(9): amidated lysine
XHHALTHAX

SEQ ID NO: 9 - protein, 9 aa, Artificial Sequence
  Synthetic Peptide
  MISC_FEATURE (1)..(1): acetylated cysteine
  MISC_FEATURE (9)..(9): amidated lysine
XAHNLTHAX

SEQ ID NO: 10 - protein, 15 aa, Artificial Sequence
  Synthetic Peptide
  MISC_FEATURE (1)..(1): acetylated glutamic acid
XNPECILDKHVQRVM

SEQ ID NO: 11 - protein, 5 aa, Artificial Sequence
  Synthetic Peptide
  MISC_FEATURE (1)..(1): acetylated alanine
  MISC_FEATURE (5)..(5): amidated lysine
XFAQX

SEQ ID NO: 12 - protein, 18 aa, Artificial Sequence
  Synthetic Peptide
CSSLDEPGRGGFSSESKV

SEQ ID NO: 13 - protein, 20 aa, Artificial Sequence
  Synthetic Peptide
CSQGTFTSDYSKYLDSRRAQ

SEQ ID NO: 14 - protein, 20 aa, Artificial Sequence
  Synthetic Peptide
CNSTKNLTFAMRSSGDYGEV

SEQ ID NO: 15 - protein, 7 aa, Artificial Sequence
  Synthetic Peptide
  MISC_FEATURE (1)..(1): acetylated lysine
  MISC_FEATURE (7)..(7): amidated histidine
XAAAACX

SEQ ID NO: 16 - protein, 6 aa, Artificial Sequence
  Synthetic Peptide
  MISC_FEATURE (1)..(1): acetylated lysine
  MISC_FEATURE (6)..(6): amidated histidine
XAAACX

SEQ ID NO: 17 - protein, 8 aa, Artificial Sequence
  Synthetic Peptide
  MISC_FEATURE (1)..(1): acetylated lysine
  MISC_FEATURE (8)..(8): amidated histidine
XAAAAACX

SEQ ID NO: 18 - protein, 7 aa, Artificial Sequence
  Synthetic Peptide
  MISC_FEATURE (1)..(1): epsilon-carboxy derivative of lysine
  MISC_FEATURE (3)..(3): 4Hyp
XAXCTAE

SEQ ID NO: 19 - protein, 6 aa, Artificial Sequence
  Synthetic Peptide
  MISC_FEATURE (1)..(1): acetylated cysteine
  MISC_FEATURE (6)..(6): amidated lysine
XDWLPX

SEQ ID NO: 20 - protein, 8 aa, Artificial Sequence
  Synthetic Peptide
  MISC_FEATURE (1)..(1): acetylated alanine
  MISC_FEATURE (8)..(8): amidated glycine
XCFALPKX

SEQ ID NO: 21 - protein, 7 aa, Artificial Sequence
  Synthetic Peptide
  MISC_FEATURE (1)..(1): acetylated lysine
  MISC_FEATURE (7)..(7): amidated cysteine
XGEAFQX

SEQ ID NO: 22 - protein, 8 aa, Artificial Sequence
  Synthetic Peptide
  MISC_FEATURE (1)..(1): acetylated glycine
  MISC_FEATURE (8)..(8): amidated lysine
XAQCAFLX

SEQ ID NO: 23 - protein, 10 aa, Artificial Sequence
  Synthetic Peptide
  MISC_FEATURE (1)..(1): acetylated alanine
  MISC_FEATURE (10)..(10): amidated serine
XKVTMTCSAX

SEQ ID NO: 24 - protein, 8 aa, Artificial Sequence
  Synthetic Peptide
  MISC_FEATURE (1)..(1): acetylated asparagine
  MISC_FEATURE (8)..(8): amidated asparagine
XYRWRCKX

SEQ ID NO: 25 - protein, 10 aa, Artificial Sequence
  Synthetic Peptide
  MISC_FEATURE (1)..(1): acetylated lysine
  MISC_FEATURE (10)..(10): amidated cysteine
XTPSPFDSHX

SEQ ID NO: 26 - protein, 9 aa, Artificial Sequence
  Synthetic Peptide
  MISC_FEATURE (1)..(1): acetylated lysine
  MISC_FEATURE (9)..(9): amidated cysteine
XSDSWHYWX

SEQ ID NO: 27 - protein, 9 aa, Artificial Sequence
  Synthetic Peptide
  MISC_FEATURE (1)..(1): acetylated cysteine
  MISC_FEATURE (9)..(9): amidated lysine
XPIEDRPMX

SEQ ID NO: 28 - DNA, 12 bp, Artificial Sequence
  Synthetic Sequence
  misc_feature (1)..(1): 5′ amino C6 linker NH2-C6
atcgatcgatcg

SEQ ID NO: 29 - protein, 9 aa, Artificial Sequence
  Synthetic Peptide
  MISC_FEATURE (1)..(1): acetylated lysine
  MISC_FEATURE (9)..(9): amidated histidine
XAAAAAACX

SEQ ID NO: 30 - protein, 15 aa, Artificial Sequence
  Synthetic Peptide
  MISC_FEATURE (1)..(1): acetylated glutamic acid
  MISC_FEATURE (15)..(15): amidated methionine
XNCPEILDKHVQRVX

SEQ ID NO: 31 - protein, 6 aa, Artificial Sequence
  Synthetic Peptide
  MISC_FEATURE (1)..(1): epsilon-carboxy derivative of lysine
  MISC_FEATURE (6)..(6): 4Hyp
XEATCX

SEQ ID NO: 32 - protein, 15 aa, Artificial Sequence
  Synthetic Peptide
  MISC_FEATURE (1)..(1): acetylated cysteine
  MISC_FEATURE (15)..(15): amidated methionine
XENPEILDHVKQRVX

SEQ ID NO: 33 - protein, 15 aa, Artificial Sequence
  Synthetic Peptide
  MISC_FEATURE (1)..(1): acetylated cysteine
  MISC_FEATURE (15)..(15): amidated methionine
XENPEILDKHVQRVX

SEQ ID NO: 34 - protein, 18 aa, Artificial Sequence
  Synthetic Peptide
  MISC_FEATURE (18)..(18): valine hydrazide
CSSLDEPGRGGFSSESKX

SEQ ID NO: 35 - protein, 20 aa, Artificial Sequence
  Synthetic Peptide
  MISC_FEATURE (20)..(20): glutamine hydrazide
CSQGTFTSDYSKYLDSRRAX

SEQ ID NO: 36 - protein, 20 aa, Artificial Sequence
  Synthetic Peptide
  MISC_FEATURE (20)..(20): valine hydrazide
CNSTKNLTFAMRSSGDYGEX

SEQ ID NO: 37 - protein, 9 aa, Artificial Sequence
  Synthetic Peptide
CPIEDRPMC